(19) United States
(12) Patent Application Publication
(10) Pub. No.: US 2017/0029531 A1
(43) Pub. Date: Feb. 2, 2017

(54) INHIBITION OF LACTATE DEHYDROGENASE 5 (LDH-5) BINDING, INCORPORATION, INTERNALIZATION AND/OR ENDOCYTOSIS TO IMMUNE CELLS

(71) Applicant: Seattle Children's Hospital (dba Seattle Children's Research Institute), Seattle, WA (US)

(72) Inventor: Courtney Crane, Seattle, WA (US)

(21) Appl. No.: 15/302,458

(22) PCT Filed: Apr. 7, 2015
(86) PCT No.: PCT/US15/24738

§ 371 Date: Oct. 6, 2016

Related U.S. Application Data
(60) Provisional application No. 61/977,500, filed on Apr. 9, 2014.

(51) Int. Cl. C07K 16/40

(52) U.S. Cl. CPC C07K 16/40; C07K 2317/55; C07K 2317/565; C07K 2317/34; C07K 2317/76

(57) ABSTRACT

Disclosed are methods of treating cancer in a subject including administering to the subject a therapeutic dose of an agent that inhibits LDH-5 binding to, incorporation, internalization and/or endocytosis into an immune cell. In some alternatives, the agent is an anti-LDH-5 antibody. Some alternatives include a step of detecting LDH-5, or a variant thereof in said subject, and administering to said subject a therapeutic dose of an anti-LDH-5 antibody raised against the amino terminus of said LDH-5 or a variant thereof.

### INCORPORATION BY REFERENCE TO ANY PRIORITY APPLICATIONS

The present application claims the benefit of priority to U.S. Provisional Patent Application No. 61/977,500, filed Apr. 9, 2014 the entire disclosure of which is incorporated herein by reference in its entirety.

### STATEMENT REGARDING FEDERALLY SPONSORED R&D

This invention was made with government support under 5K99CA151412-02, 5R00CA151412-03, and 5R00CA151412-04, awarded by National Institutes of Health, U.S. Department of Health and Human Services. The government has certain rights in the invention.

### REFERENCE TO SEQUENCE LISTING, TABLE, OR COMPUTER PROGRAM LISTING

The present application is being filed along with a Sequence Listing in electronic format. The Sequence Listing is provided as a file entitled SCRI-071WO_SEQUENCE_LISTING.TXT, created Mar. 18, 2015, which is 7 kb in size. The information is the electronic format of the Sequence Listing is incorporated herein by reference in its entirety.

### FIELD OF THE INVENTION

Disclosed are methods of treating or inhibiting cancer in a subject including administering to the subject a therapeutic dose of an agent that inhibits LDH-5 binding to, incorporation, internalization and/or endocytosis into an immune cell. In some alternatives, the agent is an anti-LDH-5 antibody. Some alternatives include a step of detecting LDH-5, or a variant thereof in said subject, and administering to said subject a therapeutic dose of an anti-LDH-5 antibody raised against the amino terminus of said LDH-5 or a variant thereof.

### BACKGROUND OF THE INVENTION

Tumor-associated myeloid cells (TAMC) have the capacity to shape the quality and duration of local immune responses, and are well-documented suppressors of effective anti-tumor immunity. Abundance of these cells in the tumor microenvironment correlates with a poor prognosis in patients with many types of solid tumors. TAMC are the most predominant leukocyte present in the brain tumor microenvironment, ranging from 40-90% of immune cells recovered from freshly isolated tissue resected from patients with GBM (Kostianovsky, A. M., Maier, L. M., Anderson, R. C., Bruce, J. N. & Anderson, D. E. Astrocytic regulation of human monocytic/microglial activation. J Immunol 181, 5425-5432 (2008); Hussain, S. F., et al. The role of human glioma-infiltrating microglia/macrophages in mediating antitumor immune responses. Neuro-oncology 8, 261-279 (2006)). Myeloid cells can prevent effective T cell activation, proliferation and survival in the tumor microenvironment (Hussain, S. F., et al., supra). These functions can be mediated by the expression of inhibitory surface proteins, such as PD-L1 (Bloch, O., et al. Gliomas promote immunosuppression through induction of B7-H1 expression in tumor-associated macrophages. Clinical cancer research: an official journal of the American Association for Cancer Research 19, 3165-3175 (2013).), and the production of immunosuppressive cytokines, such as TGFβ (Lee, H. W., Choi, H. J., Ha, S. J., Lee, K. T. & Kwon, Y. G. Recruitment of monocytes/macrophages in different tumor microenvironments. Biochimica et biophysica acta 1835, 170-179 (2013)). By the time a solid tumor is detected with even the most sophisticated imaging protocols, an immune suppressive tumor microenvironment is already well established. This is especially important in the brain tumor microenvironment, as tissue-resident myeloid cells, such as microglia and perivascular macrophages, are prevalent before transformation and can contribute to tumor immune escape of transformed cells at a very early stage (Kohanbash, G. & Okada, H. Myeloid-derived suppressor cells (MDSCs) in gliomas and glioma-development. Immunological investigations 41, 658-679 (2012); Zheng, P. P., van der Weiden, M., van der Spek, P. J., Vincent, A. J. & Kros, J. M. Isocitrate dehydrogenase 1R132H mutation in microglia/macrophages in gliomas: indication of a significant role of microglia/macrophages in glial tumorigenesis. Cancer biology & therapy 13, 836-839 (2012)), prior to compromise of the blood brain barrier and the infiltration of peripheral cells.

Lactate dehydrogenase is a tetrameric metabolic enzyme that promotes ATP production in resource-deprived and hypoxic environments through the anaerobic glycolytic pathway (Altman, M. & Robin, E. D. Survival during prolonged anaerobiosis as a function of an unusual adaptation involving lactate dehydrogenase subunits. Comparative biochemistry and physiology 30, 1179-1187 (1969); Kaplan, N. O. Lactate Dehydrogenase—Structure and Function. Brookhaven symposia in biology 17, 131-153 (1964)). There are five isoforms of LDH, consisting of different ratios of alpha and beta subunits with varied tissue specificities. In healthy individuals, the alpha only tetramer, LDH-5, represents 1-2% of total serum LDH (Kustner, W. & Weinreich, J. [Comparative studies of lactate dehydrogenase isoenzyme patterns in the serums, tumors and metastases of tumor patients]. Verhandlungen der Deutschen Gesellschaft fur Innere Medizin 75, 529-532 (1969); Beliaev, M. [Serum lactate dehydrogenase isoenzymatic spectrum with regard to age in virtually healthy persons]. Laboratornoe delo, 152-154 (1981)). The expression of LDH-5 in healthy individuals is generally restricted to the liver, and is believed to be important for local metabolism and waste management (Pettit, S. M., Nealon, D. A. & Henderson, A. R. Purification of lactate dehydrogenase isoenzyme-5 from human liver. Clinical chemistry 27, 88-93 (1981)). Although elevated serum LDH-5 correlates with a poor prognosis in a variety of cancers, the mechanism for this is not known (Giatromanolaki, A., et al. Lactate dehydrogenase 5 (LDH-5) expression in endometrial cancer relates to the activated VEGF/VEGFR2 (KDR) pathway and prognosis. Gynecologic oncology 103, 912-918 (2006); Kolev, Y., Uetake, H., Takagi, Y. & Sugihara, K. Lactate dehydrogenase-5 (LDH-5) expression in human gastric cancer: association with hypoxia-inducible factor (HIF-1alpha) pathway, angiogenic factors production and poor prognosis. Annals of surgical oncology 15, 2336-2344 (2008); and Koukourakis, M. I., Giatromanolaki, A., Sivridis, E., Gatter, K. C. & Harris, A. L. Lactate dehydrogenase 5 expression in operable colorectal cancer: strong association with survival and activated vascular endothelial growth factor pathway—a report of the Tumour Angiogenesis Research Group. J Clin Oncol 24, 4301-4308 (2006)). It has been proposed that tumor-derived LDH-5 promotes local and systemic immune suppression through directly impacting local and circulating myeloid cells in patients, causing impairing local immune responses, particularly those mediated by Natural Killer cells. Production of LDH by tumor cells and detection of LDH activity in the sera of patients with GBM also suggest that systemic effects on innate immune responses can correlate with tumor burden in these patients.

The function of NK cells is critical to the prevention of de novo tumor growth through a process known as immune surveillance (Dunn, G. P., Bruce, A. T., Ikeda, H., Old, L. J. & Schreiber, R. D. Cancer immunoediting: from immunosurveillance to tumor escape. Nat Immunol 3, 991-998 (2002); Langers, I., Renoux, V. M., Thiry, M., Delvenne, P. & Jacobs, N. Natural killer cells: role in local tumor growth and metastasis. Biologics: targets & therapy 6, 73-82 (2012)). Patients that are deficient in functional NK cells prematurely and rapidly progress through the stages of tumor development (Langers, I. et al., supra). In addition, NK cell function correlates with survival in patients with solid tumors (Zamai, L., et al. NK cells and cancer. J Immunol 178, 4011-4016 (2007); Menard, C., et al. Natural killer cell IFN-gamma levels predict long-term survival with imatinib mesylate therapy in gastrointestinal stromal tumor-bearing patients. Cancer Res 69, 3563-3569 (2009)). Despite their importance in eliminating transformed cells in the early stages of tumorigenesis, the role of the innate immune system in preventing tumor recurrence through surveillance is not considered in most immunotherapy protocols. Here, it has been demonstrated that exposure to LDH-5 induces myeloid cell expression of a subset of ligands for the activating receptor NKG2D, causing NK cell degranulation, perforin depletion, and impaired lysis of tumor cells. Based on these data, it is possible that LDH-5 and its receptor can be novel targets for therapy that will improve anti-tumor immune surveillance by NK cells. The experiments described herein provide a basis for improved treatment of previously diagnosed patients with minimal residual disease GBM. Thus an improved NK cell-mediated immune surveillance of tumor cells can delay or prevent tumor recurrence. Improved understanding of the role of LDH-5 in modulating innate immunity provides a basis for creation of novel therapies for GBM patients with newly diagnosed disease.

Therapeutic options for GBM patients are limited to surgery, chemotherapy and radiation, which can fail to resolve disease (Stupp, R., et al. Radiotherapy plus concomitant and adjuvant temozolomide for glioblastoma. N Engl J Med 352, 987-996 (2005)). Immunotherapy is appealing because it can specifically target tumor cells; however, an immunosuppressive tumor microenvironment is a significant obstacle, as clinical studies demonstrate antitumor immune responses ex vivo, without improved clinical outcomes. Tumor cells can inhibit immune responses through secretion of anti-inflammatory cytokines or recruitment of immunosuppressive cells (Rabinovich, G. A, Gabrilovich, D. & Sotomayor, E. M. Immunosuppressive strategies that are mediated by tumor cells. Annual Review of Immunology 25, 267-296 (2007)). NK cells impede tumor growth through the process of immune surveillance. NK cells recognize transformed cells that express ligands for specific NK cell activating receptors, such as NKG2D (Raulet, D. H. & Guerra, N. Oncogenic stress sensed by the immune system: role of natural killer cell receptors. Nature reviews. Immunology 9, 568-580 (2009)). In humans, there are eight ligands for NKG2D (MICA, MICB, and ULBP 1-6) and they can be expressed by any cell type undergoing stress (Raulet, D. H., Gasser, S., Gowen, B. G., Deng, W. & Jung H. Regulation of Ligands for the NKG2D Activating Receptor. Annu Rev Immunol 31, 413-441 (2013)). NKG2D's importance in immune surveillance is highlighted by the observation that NKG2D-deficient mice are more susceptible to oncogene-induced tumor development (Guerra, N., et al. NKG2D-deficient mice are defective in tumor surveillance in models of spontaneous malignancy. Immunity 28, 571-580 (2008)). Tumors are frequently infiltrated by myeloid cells that acquire immunosuppressive functions (Rabinovich, G. A, Gabrilovich, D. & Sotomayor, E. M. Immunosuppressive strategies that are mediated by tumor cells. Annual Review of Immunology 25, 267-296 (2007)). NKG2D ligands have been detected on TIMCs in tumor-bearing mice, which can be lysed by activated NK cells (Nausch, N., Galani, I. E., Schlecker, E. & Cerwenka, A Mononuclear myeloid-derived “suppressor” cells express RAE-1 and activate natural killer cells. Blood 112, 4080-4089 (2008)).

The myeloid cell lineage includes circulating monocytes in the peripheral blood and macrophages in tissues. Within tumors, tumor-infiltrating myeloid cells (TIMCs) can acquire immunosuppressive functions. Although myeloid-derived suppressor cells (MDSCs) have been documented to impair immune responses, Nausch et al. reported that the expression of the NKG2D ligand Rae-1 on MDSCs in a mouse lymphoma model system activated, rather than suppressed, the host NK cells and potentiated tumor elimination.

### SUMMARY OF THE INVENTION

Myeloid cells are key regulators of the tumor microenvironment, governing local immune responses. In some alternatives described herein, tumor-infiltrating myeloid cells and circulating monocytes in patients with glioblastoma multiforme (GBM) express ligands for activating the Natural killer group 2, member D (NKG2D) receptor, which cause down-regulation of NKG2D on natural killer (NK) cells. In an exemplary alternative, tumor-infiltrating NK cells isolated from GBM patients failed to lyse NKG2D ligand-expressing tumor cells. It is also further demonstrated that lactate dehydrogenase (LDH) isoform 5 secreted by glioblastoma cells induces NKG2D ligands on monocytes isolated from healthy individuals. Furthermore, sera from GBM patients contain elevated amounts of LDH, which correlate with expression of NKG2D ligands on their autologous circulating monocytes. NKG2D ligands also are present on circulating monocytes isolated from patients with breast, prostate, and hepatitis C virus-induced hepatocellular carcinomas. Together, these findings reveal a previously unidentified immune evasion strategy whereby tumors produce soluble factors that induce NKG2D ligands on myeloid cells, subverting antitumor immune responses.

In the alternatives described herein, surprisingly, NKG2D ligands are expressed on the cell surface of both the tumor-infiltrating myeloid cells and circulating monocytes in patients with GBM. Interactions between NKG2D ligand-bearing myeloid cells and NK cells down-modulate NKG2D on NK cells and impair their antitumor activity. Further, in an exemplary alternative it is demonstrated that lactate dehydrogenase isoform 5 (LDH5) secreted by glioblastoma cells induces the transcription and expression of NKG2D ligands in monocytes from healthy individuals and that sera from GBM patients contain elevated levels of LDH, correlating with NKG2D ligand expression on their myeloid cells.

Accordingly, aspects of the invention described herein, include methods of treating, inhibiting, and/or ameliorating cancer in a subject and can include administering to the subject a therapeutic dose of an agent that inhibits LDH-5 binding to, incorporation, internalization and/or endocytosis into an immune cell.

Some alternatives relate to a method of treating, inhibiting, or ameliorating cancer in a subject comprising administering to said subject an amount or a therapeutic dose of a binding agent (e.g., an antibody, preferably a monoclonal antibody, or binding fragment thereof, such as a Fab fragment or a fragment comprising a CDR domain) that inhibits LDH-5 binding and/or incorporation and/or internalization and/or endocytosis to an immune cell (e.g., an antibody, preferably a monoclonal antibody, or binding fragment thereof, such as a Fab fragment or a fragment comprising a CDR domain that is specific for the amino terminus of LDH-5, such as amino acid residues 1-166, 1-150, 1-125, 1-100, 1-75, 1-50, 1-25, 1-15, 1-10, or 1-5 or a range of amino acid residues in between any two numbers within positions 1-166, including 1 and 166. In some alternatives, the binding agent is an antibody. In some alternatives, the antibody is a monoclonal antibody. In some alternatives, the binding agent is a binding fragment of these antibodies. In some alternatives, the binding fragment is a Fab fragment. In some alternatives, the binding fragment comprises a CDR domain. In some alternatives, the binding fragment is specific for the amino acids within the amino terminus of LDH-5, wherein the amino acids are amino acid residues 1-166, 1-150, 1-125, 1-100, 1-75, 1-50, 1-25, 1-15, 1-10, or 1-5 or a range of amino acid residues in between any two numbers within positions 1-166, including 1 and 166. Preferably, said binding agent is specific for a peptide comprising the following sequence or an epitope present in the following peptide: ATLKDQLIYNLLKEEQTPQNKITVVGVGAVGMACAISILMKDLADELALV (SEQ ID NO: 1) or a fragment thereof comprising at least 3, 5, 7, 9, 12, 15, 17, 20, 23, 26, 29, 33, 36, 39, 43, 47, or 50 amino acids or a size that is within a number range generated by any two numbers in between 3-51 amino acids. In some alternatives, the binding agent is specific for ATLKDQLIYNLLKEEQTPQNKITVVGVGAVGMACAISILMKDLADELALV (SEQ ID NO: 1). Desirably, said binding agent is specific for the following peptide or an epitope present in the following peptide: MATLKDQLIYNLLKEEQTPQNKITVVGVGAVGMACAISILMKDLADELALVDVIEDK LKGEMMDLQHGSLFLRTPKIVSGKDYNVTANSKLVIITAGARQQEGESRLNLVQRNV NIFKFIIPNVVKYSPNCKLLIVSNPVDILTYVAWKISGFPKNRVIGSGCNLDSARFRYL MGERLGVHPLSCHGWVLGEHGDS SVPVWSGMNVAGVSLKTLHPDLGTDKDKEQW KEVHKQVVERVFTE (SEQ ID NO: 2) or a fragment thereof comprising at least residues 1-241, 1-200, 1-175, 1-150, 1-125, 1-100, 1-75, 1-50, 1-25, 1-15, 1-10, or 1-5 or a range defined by any two numbers in between 1-241, including 1 and 241. Preferably, in some alternatives, said binding agent binds to LDH-5 without inhibiting or abolishing enzymatic activity.

In some of these alternative methods, the agent is an anti-LDH-5 antibody, such as a polyclonal or monoclonal antibody. In some contexts, the anti-LDH-5 antibody, such as a polyclonal or monoclonal antibody, is specific for the amino terminus of LDH-5, which can be amino acid residues 1-51, 1-25, or 1-10 of an LDH-5 molecule and in some alternatives, said amino-terminal antibody or binding fragment thereof binds to LDH-5 without inhibiting or abolishing its enzymatic activity. In some alternatives, an agent is provided, wherein the agent is an anti-LDH-5 antibody or portion thereof, preferably a binding portion thereof. In some alternatives, the antibody is a polyclonal antibody. In some alternatives, the antibody is a monoclonal antibody or binding fragments of these antibodies.

In some of these methods, the anti-LDH-5 antibody is raised against the amino terminus of LDH-5, such as amino acid residues 1-166, 1-150, 1-125, 1-100, 1-75, 1-50, 1-25, 1-15, 1-10, or 1-5 or a range defined by any two numbers in between 1-166, including 1 and 166, e.g., of the peptides provided by SEQ. ID NOs. 1 or 2. In some alternatives the anti-LDH-5 antibody is raised against amino acid residues 1-166, 1-150, 1-125, 1-100, 1-75, 1-50, 1-25, 1-15, 1-10, or 1-5 or a range defined by any two numbers in between 1-166, including 1 and 166 of the amino terminus of LDH-5. Preferred antibodies or binding fragments thereof (e.g., Fab fragment or a fragment comprising a CDR domain) are specific for an epitope defined by ATLKDQLIYNLLKEEQTPQN (SEQ ID NO: 3) or KITVVGVGAVGMACAISILMKDLADELALV (SEQ ID NO: 4), corresponding to the N-terminal amino acids 2-21 and amino acids 22-51 of Human LDH-5, respectively. In some alternatives, antibodies or antibody binding fragments are provided, wherein the antibodies or antibody binding fragments are specific for an epitope defined by SEQ ID NO: 3 or SEQ ID NO: 4.

In some of these methods, the antibody or a binding fragment thereof (e.g., Fab fragment or a fragment comprising a CDR domain) is specific for a peptide having the amino acid sequence of SEQ ID NO: 1, 2 or 3, preferably, the amino terminus of said sequence, such as amino acid residues 1-166, 1-150, 1-125, 1-100, 1-75, 1-50, 1-25, 1-15, 1-10, or 1-5 or a range defined by any two numbers in between 1-166, including 1 and 166) of said sequences. In some alternatives, the antibody or binding fragment comprises a Fab fragment or a fragment comprising a CDR domain.

In some of these methods, the antibody comprises Ab84716 or a portion thereof, which is specific for an epitope defined by ATLKDQLIYNLLKEEQTPQNKITVVGVGAVGMACAISILMKDLADELALV (SEQ. ID. NO. 1, corresponding to N terminal amino acids 2-51 of Human LDHA.

In some of these methods, the antibody is a monoclonal antibody.

In some of these methods, the monoclonal antibody is Ab85326, also named AF14A11.

In some of these methods, the cancer is in the brain, endometrium, colon, blood, lung or epithelium/mouth.

In some of these methods, the lung cancer is non-small cell lung carcinoma (NSCLC).

In some of these methods, the cancer of the epithelium/mouth is a squamous cell carcinoma.

In some of these methods, the cancer is a glioma.

In some of these methods, the glioma is a glioblastoma.

In some of these methods, the agent inhibits LDH-5 binding and/or incorporation and/or internalization and/or endocytosis to a lymphocyte, an NK Cell, an NKG2D-expressing cell and/or a monocyte. Accordingly, aspects of the invention include methods of inhibiting LDH-5 binding and/or incorporation and/or internalization and/or endocytosis to a lymphocyte, an NK Cell, an NKG2D-expressing cell and/or a monocyte, wherein a subject is provided an amount or a therapeutic dose of an LDH-5 binding agent (e.g., an antibody, preferably a monoclonal antibody, or binding fragment thereof, such as a Fab fragment or a fragment comprising a CDR domain) that inhibits LDH-5 binding and/or incorporation and/or internalization and/or endocytosis to an immune cell (e.g., an antibody, preferably a monoclonal antibody, or binding fragment thereof, such as a Fab fragment or a fragment comprising a CDR domain that is specific for the amino terminus of LDH-5, such as amino acid residues 1-166, 1-150, 1-125, 1-100, 1-75, 1-50, 1-25, 1-15, 1-10, or 1-5 or a range defined by any two numbers in between 1-166, including 1 and 166 of SEQ ID NO. 1, 2, 3 or 4). Preferably, in some alternatives, said binding agent binds to LDH-5 without inhibiting or abolishing enzymatic activity. Preferably, said subject is identified or selected as a subject in need of an agent that inhibits LDH-5 binding and/or incorporation and/or internalization and/or endocytosis to an immune cell or a subject in need of an agent that inhibits, ameliorates, or treats cancer. Desirably, said subject is analyzed, observed, or monitored for an inhibition in cancer or a marker thereof after being provided any one or more of the aforementioned agents that inhibit LDH-5 binding and/or incorporation and/or internalization and/or endocytosis to an immune cell. In some alternatives, the subject is identified as expressing LDH-5 that is elevated compared to a control level such as the level or amount detected in a biological sample obtained from a subject without cancer or with a benign tumor.

Some alternatives relate to methods of treating or inhibiting cancer in a subject comprising detecting the presence or amount of LDH-5, or a variant thereof in said subject (e.g., this is one approach to identify or select a subject in need of an agent that inhibits LDH-5 binding and/or incorporation and/or internalization and/or endocytosis to an immune cell) and administering or providing to said subject an amount or therapeutic dose of an anti-LDH-5 antibody raised against the amino terminus of said LDH-5 or a variant thereof (e.g., an antibody, preferably a monoclonal antibody, or binding fragment thereof, such as a Fab fragment or a fragment comprising a CDR domain that is specific for the amino terminus of LDH-5, such as amino acid residues 1-166, 1-150, 1-125, 1-100, 1-75, 1-50, 1-25, 1-15, 1-10, or 1-5 or a range defined by any two numbers in between 1-166, including 1 and 166 of SEQ ID NOs 1, 2, 3 or 4). Preferably, in some alternatives, said binding agent binds to LDH-5 without inhibiting or abolishing enzymatic activity.

In some alternatives, the detection of LDH-5 comprises detection of LDH-5, a subunit of LDH-5, or variant thereof or LDH-5 enzymatic activity in a biological sample obtained from a subject.

In some alternatives, the level of LDH-5 protein or level of LDH-5 activity is elevated relative to a control level such as the level or amount detected in a biological sample obtained from a subject without cancer or with a benign tumor.

In some alternatives, LDH-5 is detected in a biological sample selected from the group consisting of a serum sample, a tumor environment/microenvironment, a tissue sample from a tumor and a tissue sample from a region surrounding a tumor.

In some alternatives, LDH-5 is detected in serum and binding of said antibody is to serum LDH-5.

In some alternatives, the antibody is administered in combination with one or more anti-cancer agents.

In some alternatives, the one or more anti-cancer agents is an antibody or binding fragment thereof selected from the group consisting of an anti-MICB antibody, an anti-ULBP1 antibody and an anti-MICA antibody. In some alternatives, the anti-MICB antibody, anti-ULBP1 antibody and/or anti-MICA antibody is humanized.

In some alternatives, the agent is an anti-LDH-5 antibody, which inhibits LDH-5 binding to a receptor on an immune cell.

Some alternatives relate to a method of treating, inhibiting, or ameliorating cancer in a subject including administering to said subject a therapeutic dose of a nucleotide sequence encoding a peptide from the amino terminus of LDH-5.

In some alternatives, the nucleotide sequence encodes a peptide having the amino acid sequence of SEQ ID NO: 1, SEQ ID NO: 2, SEQ ID NO: 3 or SEQ ID NO: 4.

In some alternatives, a method of inhibiting, ameliorating, or treating cancer in a subject is provided, wherein the method comprises selecting a subject to receive a binding agent specific for Lactate dehydrogenase 5 (LDH-5), or selecting a subject to receive an anti-cancer therapy, providing to said subject an amount of a binding agent, such as a fragment comprising a CDR domain or a Fab fragment, that is specific for LDH-5, preferably a binding agent that is specific for amino acid residues 1-241, 1-200, 1-175, 1-150, 1-125, 1-100, 1-75, 1-50, 1-25, 1-15, 1-10, or 1-5 or a range defined by any two numbers in between 1-241, including 1 and 241 of SEQ ID NOs 1, 2, 3 or 4 and optionally, observing, analyzing, monitoring, or measuring an inhibition, amelioration, or treatment of cancer in said subject. In some alternatives, the binding agent is an anti-LDH-5 antibody or binding fragment thereof, such as a fragment comprising a CDR domain or a Fab fragment, preferably a monoclonal antibody or binding fragment thereof, such as a fragment comprising a CDR domain or a Fab fragment. In some alternatives, the anti-LDH-5 antibody or binding fragment thereof is directed to the amino terminus of LDH-5, such as amino acid residues 1-166, 1-150, 1-125, 1-100, 1-75, 1-50, 1-25, 1-15, 1-10, or 1-5 or a range defined by any two numbers in between 1-166, including 1 and 166 of SEQ ID NOs 1, 2, 3 or 4. In some alternatives, the antibody is directed against a peptide having the amino acid sequence of SEQ ID NO: 1, 2, 3 or 4. In some alternatives, the antibody is Ab84716. In some alternatives, the antibody is a monoclonal antibody. In some alternatives, the monoclonal antibody is Ab85326, also named AF14A11. In some alternatives, the cancer is in the brain, endometrium, colon, blood, lung or epithelium/mouth. In some alternatives, the lung cancer is non-small cell lung carcinoma (NSCLC). In some alternatives, the cancer of the epithelium/mouth is a squamous cell carcinoma. In some alternatives, the cancer is a glioma. In some alternatives, the glioma is a glioblastoma. In some alternatives, the agent inhibits LDH-5 binding and/or incorporation and/or internalization and/or endocytosis to a lymphocyte, an NK Cell, an NKG2D-expressing cell and/or a monocyte. In some alternatives, the LDH-5 is extracellular. In some alternatives, the binding agent binds to LDH-5 without inhibiting or abolishing enzymatic activity. In some alternatives, the method further comprises administering the binding agent specific for Lactate dehydrogenase-5 with immunotherapy treatments, wherein the immunotherapy treatments modulate immune cells, wherein the immunotherapy treatments comprise at least one of checkpoint blockades, small molecule inhibitors, and/or adoptive cellular therapies. In some alternatives, the binding agent specific for Lactate dehydrogenase-5 is combined with checkpoint blockade immunotherapeutics. In some alternatives, the checkpoint blockade immunotherapeutics comprises anti-PD-1 or PD-L1 antibodies.

In some alternatives, a method of treating, inhibiting, or ameliorating cancer in a subject is provided, wherein the method comprises detecting LDH-5, or LDH-5 activity in a biological sample obtained from said subject, and administering to said subject a therapeutic dose of an anti-LDH-5 antibody or binding fragment thereof raised against the amino terminus of said LDH-5 or a variant thereof such as, amino acid residues 1-241, 1-200, 1-175, 1-150, 1-125, 1-100, 1-75, 1-50, 1-25, 1-15, 1-10, or 1-5 or a range defined by any two numbers in between 1-241, including 1 and 241 of SEQ ID NOs 1, 2, 3 or 4. In some alternatives, the detection of LDH-5 is detection of LDH-5 itself, a subunit of LDH-5, or LDH-5 enzymatic activity. In some alternatives, the level or amount of LDH-5 protein or level or amount of LDH-5 activity in the biological sample is elevated relative to a control such as a biological sample obtained from a subject without cancer or with a benign tumor. In some alternatives, LDH-5 is detected in a biological sample selected from the group consisting of a serum sample, a tumor environment/microenvironment, a tissue sample from a tumor and a tissue sample from a region surrounding a tumor. In some alternatives, LDH-5 is detected in serum and binding of said antibody is to serum LDH-5. In some alternatives, the antibody or binding fragment thereof is administered in combination with one or more other anti-cancer agents. In some alternatives, said one or more anti-cancer agent is an antibody selected from the group consisting of an anti-MICB antibody, an anti-ULBP1 antibody and an anti-MICA antibody. In some alternatives, the agent is an anti-LDH-5 antibody, which inhibits LDH-5 binding to a receptor on an immune cell. In some alternatives, the one of more anti-cancer agent is an LDH-5 intracellular enzymatic inhibitor and/or an anti-NKG2d antibody, wherein the anti-NKG2d antibody blocks binding and/or ULBP1. In some alternatives, the method further comprises administering the anti-LDH-5 antibody or binding fragment thereof raised against the amino terminus of said LDH-5 or a variant thereof, with immunotherapy treatments, wherein the immunotherapy treatments modulate immune cells, wherein the immunotherapy treatments comprise at least one of checkpoint blockades, small molecule inhibitors, and/or adoptive cellular therapies. In some alternatives, the anti-LDH-5 antibody or binding fragment thereof raised against the amino terminus of said LDH-5 or a variant thereof, is combined with checkpoint blockade immunotherapeutics. In some alternatives, the checkpoint blockade immunotherapeutics comprises anti-PD-1 or PD-L1 antibodies.

In some alternatives, a method of treating, inhibiting, or ameliorating cancer in a subject is provided, wherein the method comprises administering to said subject a therapeutic dose of a nucleotide sequence encoding a peptide from the amino terminus of LDH-5. In some alternatives, the nucleotide sequence encodes a peptide having the amino acid sequence of SEQ ID NO: 1, SEQ ID NO: 2, SEQ ID NO: 3 or SEQ ID NO: 4.

In some alternatives, a method of treating, inhibiting, or ameliorating cancer in a subject is provided, wherein the method comprises detecting LDH-5, or LDH-5 activity in a biological sample obtained from said subject, administering to said subject a therapeutic dose of an anti-LDH-5 antibody or binding fragment thereof raised against the amino terminus of said LDH-5 or a variant thereof such as, amino acid residues 1-241, 1-200, 1-175, 1-150, 1-125, 1-100, 1-75, 1-50, 1-25, 1-15, 1-10, or 1-5 or a range defined by any two numbers in between 1-241, including 1 and 241 of SEQ ID NOs 1, 2, 3 or 4 and administering one or more other anti-cancer agents. In some alternatives, the detection of LDH-5 is detection of LDH-5 itself, a subunit of LDH-5, or LDH-5 enzymatic activity. In some alternatives, the level or amount of LDH-5 protein or level or amount of LDH-5 activity in the biological sample is elevated relative to a control such as a biological sample obtained from a subject without cancer or with a benign tumor. In some alternatives, LDH-5 is detected in a biological sample selected from the group consisting of a serum sample, a tumor environment/microenvironment, a tissue sample from a tumor and a tissue sample from a region surrounding a tumor. In some alternatives, LDH-5 is detected in serum and binding of said antibody is to serum LDH-5. In some alternatives, one or more anti-cancer agent is an antibody selected from the group consisting of an anti-MICB antibody, an anti-ULBP1 antibody and/or an anti-MICA antibody. In some alternatives, the method further comprises administering the anti-LDH-5 antibody or binding fragment thereof raised against the amino terminus of said LDH-5 or a variant thereof, with immunotherapy treatments, wherein the immunotherapy treatments modulate immune cells, wherein the immunotherapy treatments comprise at least one of checkpoint blockades, small molecule inhibitors, and/or adoptive cellular therapies. In some alternatives, the anti-LDH-5 antibody or binding fragment thereof raised against the amino terminus of said LDH-5 or a variant thereof, is combined with checkpoint blockade immunotherapeutics. In some alternatives, the checkpoint blockade immunotherapeutics comprises anti-PD-1 or PD-L1 antibodies.

In some alternatives, a method of stimulating or inducing an immune system in a subject with cancer is provided, wherein the method comprises selecting a subject to receive a binding agent specific for Lactate dehydrogenase 5 (LDH-5), or selecting a subject to receive an anti-cancer therapy, providing to said subject an amount of a binding agent, such as a fragment comprising a CDR domain or a Fab fragment, that is specific for LDH-5, preferably a binding agent that is specific for amino acid residues 1-241, 1-200, 1-175, 1-150, 1-125, 1-100, 1-75, 1-50, 1-25, 1-15, 1-10, or 1-5 or a range defined by any two numbers in between 1-241, including 1 and 241 of SEQ ID NOs 1, 2, 3 or 4, preferably said binding agent binds to LDH-5 and optionally, observing, analyzing, monitoring, or measuring an inhibition, amelioration, or treatment of cancer in said subject. In some alternatives, the binding agent is an anti-LDH-5 antibody or binding fragment thereof, such as a fragment comprising a CDR domain or a Fab fragment, preferably a monoclonal antibody or binding fragment thereof, such as a fragment comprising a CDR domain or a Fab fragment. In some alternatives, the anti-LDH-5 antibody or binding fragment thereof is directed to the amino terminus of LDH-5, such as amino acid residues 1-166, 1-150, 1-125, 1-100, 1-75, 1-50, 1-25, 1-15, 1-10, or 1-5 or a range defined by any two numbers in between 1-166, including 1 and 166 of SEQ ID NOs 1, 2, 3 or 4. In some alternatives, the antibody is directed against a peptide having the amino acid sequence of SEQ ID NO: 1, 2, 3 or 4. In some alternatives, the antibody is Ab84716. In some alternatives, antibody is a monoclonal antibody. In some alternatives, the monoclonal antibody is Ab85326, also named AF14A11. In some alternatives, the cancer is in the brain, endometrium, colon, blood, lung or epithelium/mouth. In some alternatives, the lung cancer is non-small cell lung carcinoma (NSCLC). In some alternatives, the cancer of the epithelium/mouth is a squamous cell carcinoma. In some alternatives, the cancer is a glioma. In some alternatives, the agent inhibits LDH-5 binding and/or incorporation and/or internalization and/or endocytosis to a lymphocyte, an NK Cell, an NKG2D-expressing cell and/or a monocyte. In some alternatives, the LDH-5 is extracellular. In some alternatives, the antibody or binding fragment thereof is administered in combination with one or more other anti-cancer agents. In some alternatives, the one of more anti-cancer agent is an LDH-5 intracellular enzymatic inhibitor and/or an anti-NKG2d antibody, wherein the anti-NKG2d antibody blocks binding and/or ULBP1.

### BRIEF DESCRIPTION OF THE DRAWINGS

FIG. 1: Tumor-derived proteins induce expression of NKG2D ligands MICA/B and ULBP-1 on circulating and tumor-infiltrating myeloid cells. (Panel A) Circulating PBMC (Circ) and tumor-infiltrating leukocytes (TI) were isolated from patients with recurrent glioblastoma (GBM) (n=33) or meningioma (MNG, n=14) and stained with fluorochrome-conjugated antibodies to CD45, CD11b, MICA/B, and ULBP-1. After gating on CD45-positive cells, CD11b-positive cells were evaluated for their expression of ULBP-1 (Top panels) and MICA/B (Bottom panels). (Panel B) Mean Fluorescence Intensity (MFI) of cells stained for MICA/B (left) and ULBP-1 (right) on CD45+, CD11b+ cells in CD11b^{hi} and CD11b^{lo} GBM TIMCs (n=19) and circulating monocytes in GBM patient (n=33) and healthy control subjects (n=16). (Panel C) mRNA from CD14+ monocytes isolated from MNG (n=14) or GBM (n=19) was reverse transcribed and analyzed by quantitative PCR (qPCR) for MICA, MICB, ULBP-1, and ULBP-2. (Panel D) U87 cell-free supernatant (left) was cultured with CD14-selected MHD (Monocytes from Healthy Donors) for 48 hours and expression of MICA/B and ULBP-1 proteins were analyzed using flow cytometry. Ten thousand U87 cells were cultured in a Transwell system with CD14-selected MHD for 48 h. Monocytes were then analyzed for MICA/B and ULBP-1 expression using flow cytometry. Dashed open histograms represent isotype-matched control IgG staining, tinted histograms represent monocytes cultured in the absence of U87 supernatant (left) or U87 cells (right), open histograms represent monocytes cultured with U87 supernatant (left) or U87 cells (right). (Panel E) U87 cell supernatant was either added directly to CD14-selected MHD or heated to 95° C. for 10 minutes to denature proteins. Forty-eight hours later, monocytes were analyzed by flow cytometry for MICA/B and ULBP-1. (Panel F) U87 cell supernatant was dialyzed to remove molecules <10 kDa and fractions were added to MHD. Forty-eight hours later, monocytes were analyzed using flow cytometry for protein expression of MICA/B and ULBP-1.

FIG. 2. Biochemical purification of tumor-derived Lactate Dehydrogenase, expression in glioma cell lines and verification of LDH-mediated induction of NKG2D ligands on monocytes. (Panel A) Protein purification strategy. Five hundred mg of U87 glioblastoma cell line supernatant were subjected to ammonium sulfate precipitation. Active fractions were passed over a phenyl Sepharose column and active fractions were then subjected to size exclusion (Superdex 200), followed by ion exchange (MonoS) before Mass Spectroscopy (MS) analysis. An example of the fractionation process is shown in FIG. 8. (Panel B) Following separation by chromatography, fractions were dialyzed into PBS and cultured with MHD (healthy monocytes) for 18 hours. Whole cell mRNA was reverse transcribed, and induction of MICB and ULBP-1 was analyzed relative to media alone controls. Western blot analysis using a monoclonal antibody to LDH-5 (ab101562) was performed on 10 mg of total protein to confirm the presence of LDH-5 in the active fractions. (Panel C) mRNA isolated from PBMC of healthy donors, 293T cells (as negative controls), and U87, U251, and SF767 glioma cell lines were analyzed for expression of LDHA and LDHB by qPCR. (Panel D) A 1:10 dilution of fresh, unfrozen glioblastoma cell line supernatants was analyzed for total LDH enzymatic activity and expressed as OD 495 nm. (Panel E) Validation of LDH induction of NKG2D ligands in monocytes. Six hundred twenty five U/L of purified, native LDH-5, fresh U87 supernatant, or fresh U87 supernatant in the presence of 100 mM sodium oxamate (NaOx) were cultured with MHD. Eighteen hours later, mRNA was isolated and analyzed for MICA, MICB, and ULBP-1 by qPCR. Values represent induction relative to MHD cultured in media alone. (Panel F) Fresh U87 supernatant, 625 U/L of purified, native LDH-5, LDH-5+0.5 mM Pyruvate (Pyr), or LDH-5+0.5 mM pyruvate and 100 mM NaOx were cultured with MHD. Eighteen hours later, mRNA was isolated and analyzed for MICA, MICB and ULBP-1 by qPCR. Values represent induction relative to MHD cultured in media alone. (Panel G) MHD were cultured with native LDH-5 isolated from liver cells. Eighteen hours later, MICB (black bars) and ULBP-1 (gray bars) were measured by qPCR. (Panel H) Expression of NKG2D and NKp46 on primary human NK cells following a 12 hour incubation with LDH (50 ug/mL) or lactate (20 mM). Cells were surface stained with antibodies directed against CD3, CD56, NKG2D, and NKp46 and analyzed by flow cytometry. The expression level of NKG2D and NKp46 are plotted against CD56. (Panel I) Expression of CD107a on primary human NK cells following IL-2 (1000 U/ml) activation and a 6 hour incubation with LDH (50 ug/mL) or lactate (20 mM). Cells were surface stained with antibodies directed against CD3, CD56, and CD107a and analyzed by flow cytometry. Representative data are shown in the top panels, a summary of percent CD107a+NK cells is shown below. (Panel J) 293T cells were transiently transfected with a pcDNA3.1 (−) vector encoding either LDHA or LDHB, or both constructs. Supernatant was then cultured with CD14-positively selected MHD. Eighteen hours later, MICB and ULBP-1 were measured by qPCR. Values represent induction relative to monocytes cultured in media alone. *P<0.05, **P<0.01.

FIG. 3: Circulating monocytes and tumor-associated NKG2D ligand-expressing myeloid cells impair NK cell recognition and lysis of GBM tumor cells. (Panel A) NKG2D ligand-expressing monocytes induced NK cell degranulation and cytokine production in a NKG2D-dependent fashion. Circulating monocytes were selected from patients with MNG or GBM and co-cultured with either autologous NK cells (top), the NKL cell line (middle rows), or NKL cells plus a NKG2D neutralizing antibody (bottom row) for 3 hours in the presence of PE-conjugated anti-CD107a and brefeldin A. Cells were then stained for CD56, CD11b, and IFNγ. Plots represent CD56-gated populations. (Panel B) IL-2 activated circulating NK cells from GBM patients were cultured at a 1:1 ratio with U87 tumor cell targets, autologous TIMCs, or both for 18 hours in the presence of phycoerythrin (PE)-conjugated anti-CD107a and Brefeldin A. Cells were analyzed by flow cytometry for degranulation and IFNγ production. (Panel C) Summary of degranulation of circulating NK cells is shown in B for GBM patients (n=4). (Panel D) Freshly isolated or NK cells cultured for 24 h in 500 U/mL IL-2 isolated from healthy donors were cultured 1:1 with autologous healthy monocytes treated with control medium or with healthy monocytes pretreated for 24 h with U87 cellfree supernatant to induce NKG2D ligands (S/N mono) or were cultured 1:1:1 with healthy monocytes treated with control medium or with U87 supernatant and U87 tumor cells, and in the presence or absence of NKG2D blocking antibody. Target cell death was analyzed by staining with fluorochrome-conjugated annexin V. (Panel E) Increasing ratios of U87 supernatant-treated MHD were cultured with autologous IL-2-activated NK cells for 18 hours in the presence of PE conjugated anti-CD107a and analyzed for NK cell degranulation using flow cytometry. (Panel F) Increasing ratios of U87 supernatant-treated MHD were cultured with autologous IL-2-activated NK cells for 18 hours. NKG2D MFI was analyzed by flow cytometry on NK cells (CD3+, CD56−). (Panel G) Monocytes treated with control medium or U87 supernatant-treated monocytes (tumor-conditioned) isolated from healthy donors were co-cultured with U87 tumor cells and autologous IL-2-activated NK cells for 18 h at the indicated ratios. Target apoptosis was measured using annexin V staining of either U87 cells (gray bars) or monocytes (black bars). *P<0.05. (Panel H) Circulating and tumor-associated NK cells were isolated from patients with GBM, stained for intracellular perforin and granzyme B, and analyzed by flow cytometry (n=7). (Panel I) Circulating and tumor-associated NK cells were isolated from patients with GBM (n=4), and cultured with or without IL-2 (500 U/ml) for 24 hours. Perforin and granzyme B expression was measured by qPCR. (Panel J) Tumor-associated NK cells were isolated and cultured with IL-2 for 72 hours. Perforin content was analyzed at 18, 24, 48, and 72 hours. Autologous circulating NK cells were used as a positive control, and freshly isolated tumor-associated NK cells in the absence of IL-2 for 24 hours as a negative control. (Panel K) Tumor-associated NK cells were cultured with 500 U/ml IL-2 for 24 hours and then cultured with U87 tumor cell targets for 18 hours in the presence of PE-conjugated anti-CD107a and Brefeldin A. Cells were then analyzed by flow cytometry for degranulation and perforin content.

FIG. 4: Freshly isolated GBM patient sera contain active LDH and induce NKG2D ligands, which decrease following a reduction in tumor burden. (Panel A) Freshly isolated (nonfrozen) sera from four patients with GBM and healthy donors (n=10) were analyzed for total active LDH isoenzymes and analyzed for colorimetric change at OD495 nm. (Panel B) Monocytes isolated from a healthy donor were incubated with freshly isolated (nonfrozen) sera from four patients with GBM or with U87-conditioned supernatant. Eighteen hours later MICA, MICB, and ULBP-1 were analyzed by qPCR. mRNA expression is shown relative to monocytes cultured in medium alone. (Panel C) Flow cytometry analysis of MICA/B and ULBP-1 surface protein expression on circulating monocytes (CD45+, CD11b+, HLA-DR+) from a patient before surgical resection of tumor and 33 d after gross total tumor resection. (Panel D) Longitudinal analysis of MICA/B and ULBP-1 expression using flow cytometry on circulating monocytes in recurrent GBM patients following tumor resection. (Panel E) PBMCs were isolated from patients with hepatocellular carcinoma (HCC), prostate cancer, or breast cancer or from healthy control subjects. CD45+CD11b+ monocytes were analyzed for MICA/B and ULBP-1 expression by flow cytometry and displayed as percent positive relative to isotype-matched Ig controls (Upper). Patients with a percentage of MICB and ULBP-1 expressing monocytes greater than the mean percentage of control patients were then analyzed for mean fluorescence intensity of MICB and ULBP-1 expressing monocytes (Lower).

FIG. 5: Innate immune cell infiltration of glioblastoma tissue. (Panel A) Following collagenase digestion of freshly isolated tumor tissue, circulating PBMCs and tumor-infiltrating leukocytes were gated, stained for CD45, CD3, and CD56, and analyzed by flow cytometry. Plots represent CD45-gated cells. (Panel B) CD3−CD56+ PBMCs and tumor-infiltrating lymphocytes isolated from a representative GBM patient were stained for NKG2D before surgery and 34 d following a >90% reduction in tumor burden. (Panel C) Tumor-infiltrating lymphocytes were isolated from patients with meningioma (MNG, n=13) or GBM (n=18) and were stained for CD45, HLA-DR, and CD11b and analyzed by flow cytometry. Dot plots represent CD45+-gated cells.

FIG. 6: Circulating and tumor-infiltrating myeloid cells isolated from patients with newly diagnosed GBM express NKG2D ligands, independently of steroids, chemotherapy, and radiation. Tumor-infiltrating myeloid cells and circulating monocytes were isolated from patients before surgical or therapeutic interventions (n=4) and were analyzed for the expression of MICA/B (Upper) and ULBP-1 (Lower). Dot plots represent gating on CD45+CD11b+ cells.

FIG. 7. NKG2D ligands are not passively acquired by non-specific binding to monocytes. Monocytes were isolated from patients with GBM (n=2), and washed in HCl-acidified media (pH 3.5) for 5 minutes, and then washed. Monocytes were then stained with antibodies for CD45, CD11b, HLA-DR, and β2-microglobulin (left), a protein component of the MHC class I complex that is removed during acid washing, MICA/B, and ULBP-1 and analyzed by flow cytometry.

FIG. 8. Representative MonoS column fractionation. Following Ammonium Sulfate Precipitation, Phenyl Sepharose hydrophobicity and Supredex 200 size exclusion chromatography, active fractions were applied to a MonoS Column for ion exchange chromatography.

FIG. 9. LDH activity dose response. Fresh, native purified LDH-5 was analyzed for enzymatic activity for use as a historical standard curve for subsequent LDH activity assays. Establishment of a standard curve was performed because activity assays measure all isoforms, and a standard curve is established to correlate protein concentration with activity to use as a metric for comparison for all other LDH5 experiments. In each, the protein concentration and activity can be back calculated.

FIG. 10. LDH activity decreases following freeze-thaw. U87 supernatant was frozen at −80° C. for 48 hours, thawed and cultured with MHD for 18 hours. Monocytes were then tested for induction of MICA, MICB, and ULBP-1 by RT-qPCR. (Panel A) Primary monocytes with frozen U87 media (Panel B) Primary monocytes with fresh U87 (Panel C) Table showing the percent activity retained after freeze thaw.

FIG. 11. mRNA expression was examined in Glioblastoma cell lines. As shown, Glioblastoma cell lines produce LDH-5 subunit mRNA (Panel A) and active LDH enzyme as seen by the absorbance at 495 nm (Panel B). As shown, tumor cells express LDH5 mRNA and protein which supports that the LDH5 found in sera and patient samples is tumor derived.

FIG. 12. LDH-5 activity is significantly reduced following freeze/thaw. This indicates that LDH5 is labile, making it important to assay monomers and not just activity in patient biological fluids, or to analyze immediately, as activity is lost after freezing patient sera.

FIG. 13. Neither LDH-5 nor soluble factors produced by the glioblastoma cell line U87 is sufficient to elevate extracellular lactate. As shown, the impact that is seen on tumor cells and immune cells is not the result of elevated lactate, as has been suggested (Hussain et al, Journal of immunology, 2013).

FIG. 14. LDH-5 Expression is Up-Regulated in Hypoxia. As hypoxia elevates LDH5 expression, this suggests that autocrine functions of LDH5 may be important for survival of tumor cells in oxygen deprived environments.

FIG. 15. Patient sera analyzed immediately after tumor resection (never frozen) contains active LDH as compared to healthy donors. When freshly isolated (never frozen) patient sera were analyzed, elevated LDH5 was found in circulation, and this was correlated with the impact on monocytes.

FIG. 16. Tumor cells internalize LDH-5. (Please elaborate on figures experimental (how this was performed) and results)

FIG. 17. Tumor cells internalize LDH-5, which can be reduced by antibody blocking. 10 ug/ml of LDH5 specific monoclonal antibody was added to cells 1 hour prior to culture with LDH5 as described above and evaluated for internalization using flow cytometry to evaluate fluorescence.

FIG. 18. Sodium Oxamate, a pyruvate substrate analog, reduces internalization in tumor cells.

FIG. 19. LDH-5 internalization is receptor mediated and Clathrin dependent. LDH5 internalization is reduced with receptor mediated endocytosis inhibitors.

FIG. 20. Supernatants from 293T cells and U87, U251, and SF767 glioma cell lines were cultured on monocytes from healthy donors. Eighteen hours later, mRNA was isolated, reverse transcribed, and evaluated for ULBP-1 and MICB mRNA expression relative to monocytes cultured in medium alone. *P<0.05, **P<0.01.

FIG. 21. Purified native LDH5 or LDH1 was cultured with monocytes from healthy donors. Eighteen hours later, MICB and ULBP-1 were measured by qPCR. Values represent induction relative to monocytes cultured in medium alone.

FIG. 22. NK cells from healthy donors have decreased NKG2D expression following co-culture with autologous NKG2D ligand-expressing monocytes. Monocytes from healthy donors were isolated by CD14+ cell selection using antibody-coated magnetic beads and were cultured with U87 supernatant for 48 hours to induce NKG2D ligand expression. Autologous peripheral blood NK cells were enriched using negative selection and cultured overnight with IL-2 (500 U/mL). NK cells were cultured alone (shaded histogram) or with monocytes treated with U87-conditioned medium (open histogram) for 24 hours, and NK cells were stained for CD3, CD56, and NKG2D. Histograms represent cells gated on CD3−CD56+ cells relative to isotype-matched control Ig staining (dashed histogram).

FIG. 23. LDHa antibody blocks LDH5 uptake. Fluorescently labeled LDH5 was pre-incubated with anti-LDHa at equilmolar concentrations. The antibody protein combination was then fed to monocytes, or monocyte derived macrophages for 30 min. Cells were then trypsinized, acid washed, fixed in 2% PFA. Fluorescence intensity was obtained by flow cytometry. Anti-LDHa blocks uptake by approximately 50%. Macrophages N=1, Monocytes N=2.

FIG. 24. Clathrin mediated endocytosis inhibitor chlorpromazine blocks LDH5 uptake. Monocyte derived macrophages were pre-incubated with 25 uM chlorpromazine for 30 min. Fluorescently labeled LDH5 was then added for an additional 30 min. Cells were then trypsinized, acid washed, fixed in 2% PFA. Fluorescence intensity was obtained by flow cytometry. N=2

FIG. 25. Malate Dehydrogenase (MDH) blocks LDH5 uptake in CD14+ cells from healthy donors. Fluorescently labeled LDH5 was incubated with U87 in the presence or absence of equimolar MDH for 30 min. Cells were then trypsinized, acid washed, fixed in 2% PFA. Fluorescence intensity was obtained by flow cytometry. N=2.

FIG. 26. U87 uptake. LDHa antibody blocks LDH5 uptake. Fluorescently labeled LDH5 and Transferrin were pre-incubated with anti-LDHa at equilmolar concentrations. The antibody protein combination were then fed to U87 for 30 min. Cells were then trypsinized, acid washed, fixed in 2% PFA. Fluorescence intensity was obtained by flow cytometry. Anti-LDHa blocks uptake by approximately 50%, but has no effect on Transferrin uptake. N=2, p<0.05.

FIG. 27. U87 uptake. Oxamate blocks LDH5 uptake. Fluorescently labeled LDH5 and Transferrin were incubated with U87 in the presence or absence of 20 mM oxamate for 30 min. Cells were then trypsinized, acid washed, fixed in 2% PFA. Fluorescence intensity was obtained by flow cytometry. N=1.

FIG. 28. U87 uptake. Malate Dehydrogenase (MDH) blocks LDH5 uptake. Fluorescently labeled LDH5 and Transferrin were incubated with U87 in the presence or absence of equimolar MDH for 30 min. Cells were then trypsinized, acid washed, fixed in 2% PFA. Fluorescence intensity was obtained by flow cytometry. N=1.

FIG. 29. U87 uptake. Receptor mediated endocytosis inhibitors Dyngo 4a and Pitstop block LDH5 uptake. Cells were pre-incubated with 30 uM Dyngo 4a or 18 uM Pitstop. Fluorescently labeled LDH5 was then added for 30 min. Cells were then trypsinized, acid washed, fixed in 2% PFA. Fluorescence intensity was obtained by flow cytometry.

FIG. 30. U87 mitochondrial function. LDH5 depolarizes mitochondrial membrane. U87 were treated overnight with LDH5 then with controls 100 uM FCCP (depolarizer) or 200 ng/mL staurosporine (hyperpolarizer) for 10 min. Cells were then loaded with the mitochondrial membrane potential sensitive dye Mitotracker CMXRos for 15 min then fixed in 2% PF. Fluorescence intensity was measured by flow cytometry.

FIG. 31. U87 mitochondrial function. LDH5 does not change mitochondrial surface area. U87 were treated overnight with LDH5. Cells were fixed in 2% PF and immunostained with an anti-mitochondrial antibody and phalloidin (actin) Mitochondrial and cell surface area were calculated by Nuance software.

FIG. 32. U87 mitochondrial function. LDH5 does not reduce viability, nor rescue from staurosporine induced apoptosis. U87 were treated overnight with LDH5 then with 200 ng/mL staurosporine for 5 or 24 hours. Cells were then trypsinized, stained with Annexin 594 and analyzed by flow cytometry.

FIG. 33. Human PBMC were isolated from healthy human blood via Ficoll gradient. PBMC were cultured for 24 h in RPMI+10% FBS supplemented with 1000 U/mL IL-2. Experimental wells were pulsed with 10 ug/mL LDH-A488 for 30 min (control wells received no LDH) at 37° C. Cells were treated for 2 min with 0.5M acetic acid+150 mM NaCl (pH 2.5), followed by 4 subsequent washes in RPMI+10% FBS. Cells were stained with antibodies directed against CD4, CD8, CD28, and CD95 to delineate T cell subsets (CD28+CD95−, Naïve, Na; CD28+CD95+, central memory, CM; CD28−CD95+, effector memory, EM) or against CD3, CD56, and CD16 to delineate NK cell subsets (CD3-CD56hiCD16−; CD3−CD56dimCD16−; CD3−CD56dimCD16+). Frequency and MFI of A488 was corrected against control (no LDH pulse). Shown are 10 data points: 2 patients on 2 different days (in duplicate), and 1 patient on one day in duplicate.

FIG. 34. Human PBMC were isolated from healthy human blood via Ficoll gradient. PBMC were cultured for 24 h in RPMI+10% FBS supplemented with 1000 U/mL IL-2. Experimental wells were pulsed with 10 ug/mL LDH-A488 for either 30 min or 24 h at 37° C. Cells were treated for 2 min with 0.5M acetic acid+150 mM NaCl (pH 2.5), followed by 4 subsequent washes in RPMI+10% FBS. Cells were stained with antibodies directed against CD4, CD8, CD28, and CD95 to delineate T cell subsets (CD28+CD95−, Naïve, Na; CD28+CD95+, central memory, CM; CD28−CD95+, effector memory, EM) or against CD3, CD56, and CD16 to delineate NK cell subsets (CD3−CD56hiCD16−; CD3−CD56dimCD16−; CD3−CD56dimCD16+). Frequency and MFI of A488 was corrected against control (no LDH pulse). Shown are data from 2 patients in duplicate, each ran on a different day.

FIG. 35. Human PBMC were isolated from healthy human blood via Ficoll gradient. T cells were separated from bulk PBMC via flow-through from a CD14+ selection kit (EasySep), followed by a Pan T cell Negative selection kit (Miltenyi). T cells were cultured for 24 h in RPMI+10% FBS and stimulated with either 1000 U/mL IL-2 or Human CD3/CD28 Dynabeads (Life Technologies). Activated cells were then pulsed with or without 10 ug/mL LDH-A488 for 30 min. Cells were treated for 2 min with 0.5M acetic acid+150 mM NaCl (pH 2.5), followed by 4 subsequent washes in RPMI+10% FBS. Cells were stained with antibodies directed against CD4, CD8, CD28, and CD95 to delineate T cell subsets (CD28+CD95−, Naïve, Na; CD28+CD95+, central memory, CM; CD28−CD95+, effector memory, EM) or against CD3, CD56, and CD16 to delineate NK cell subsets (CD3-CD56hiCD16−; CD3−CD56dimCD16−; CD3−CD56dimCD16+). Frequency and MFI of A488 was corrected against control (no LDH pulse). Shown is data from 2 patients in duplicate on same day.

### DETAILED DESCRIPTION

Disclosed herein are agents that inhibit LDH-5 binding and/or incorporation and/or internalization and/or endocytosis to an immune cell, and the use of the agents to prevent down-regulation of immune responses by tumor cells.

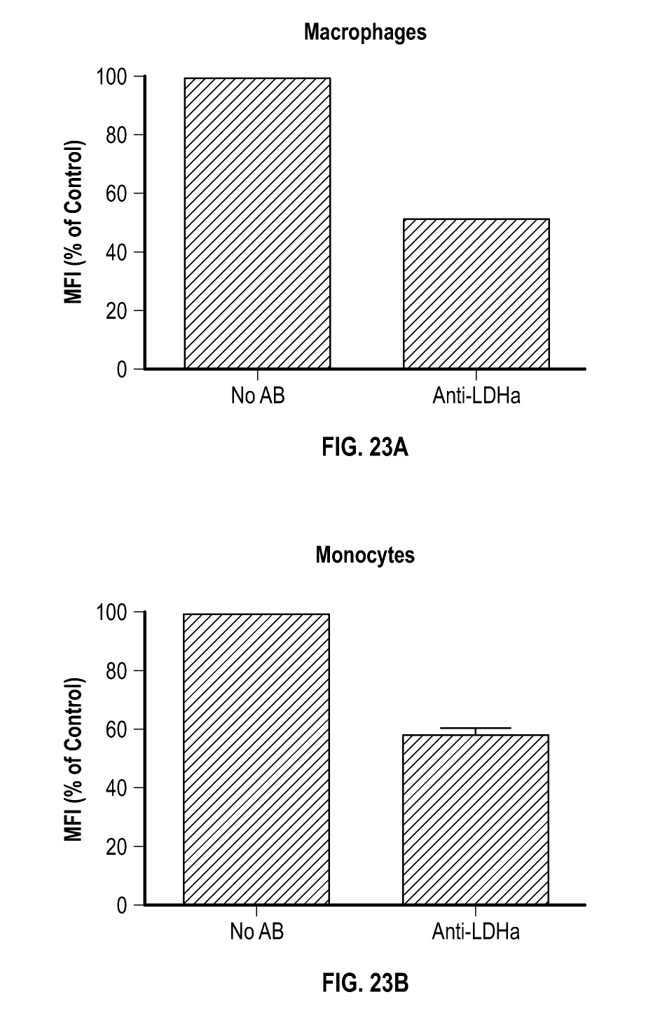

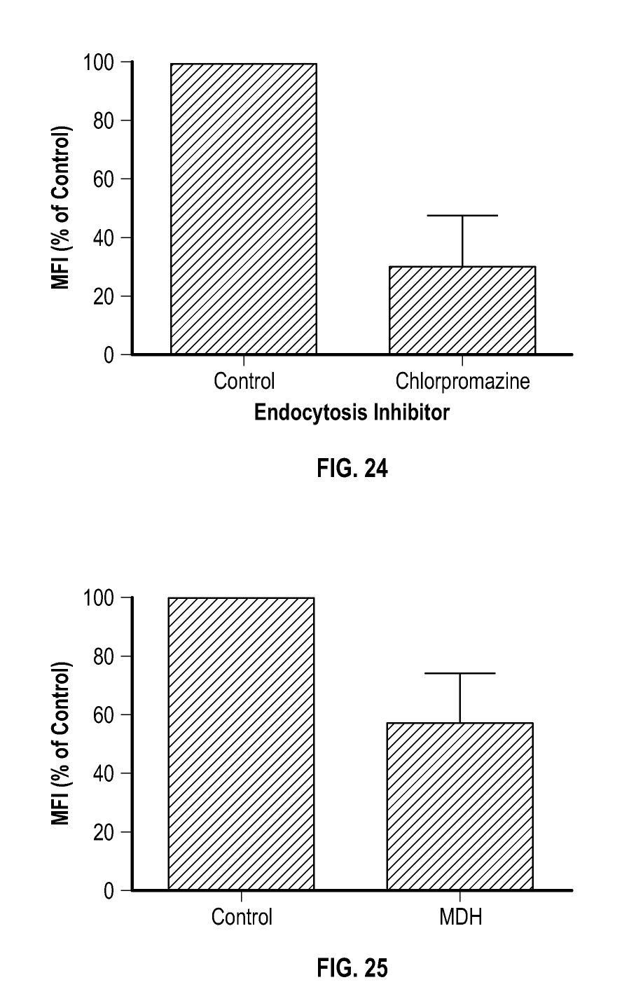

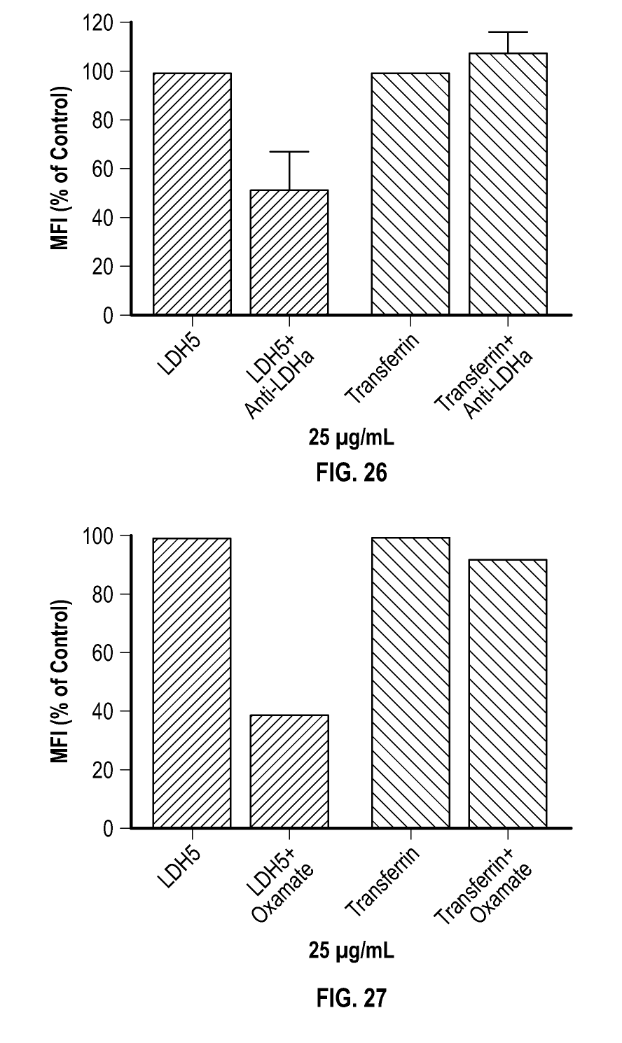

LDH-5, but not lactate, induces IL-6 gene expression in the glioblastoma cell line U87, demonstrating an impact of LDH-5 on an immune response with respect to cytokines (FIG. 26).

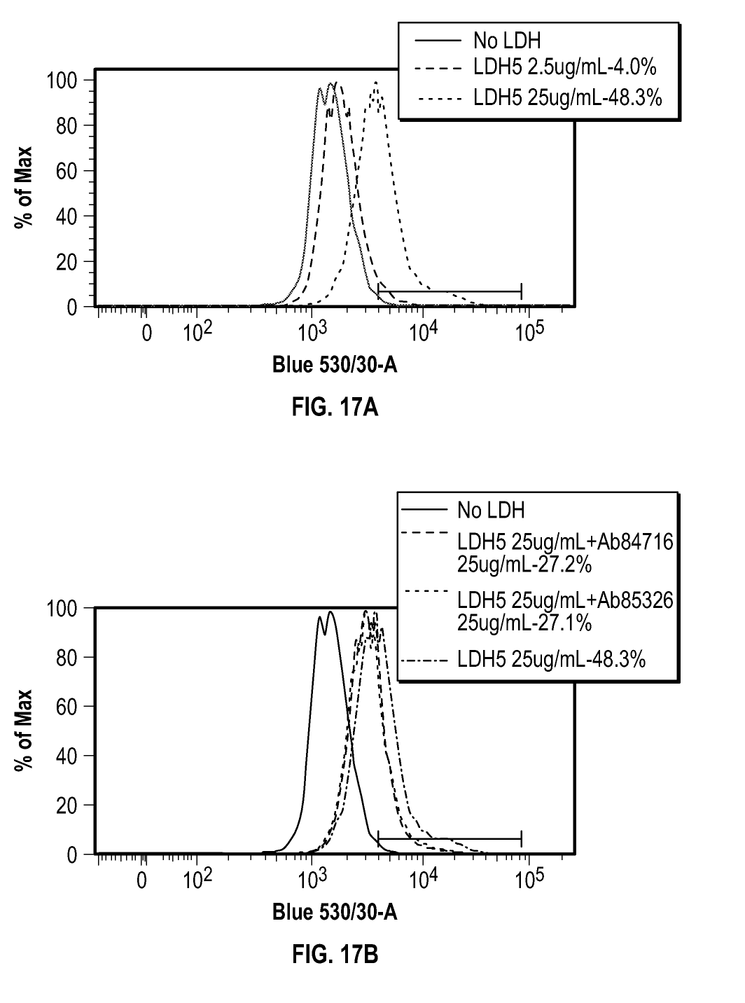

Evidence is provided herein, that by blocking incorporation of LDH-5 into a tumor itself an effect on down-regulation of the immune system (FIGS. 16-18) takes place. In some aspects, therapeutic binding agents inhibit incorporation, binding, or internalization into a tumor.

### Antibodies.

Antibodies specific for LDH-5 block down-regulation of the immune system by LDH-5. For example, one antibody preparation that blocks down-regulation of the immune system by LDH-5 is anti-LDHA antibody (ab84716; Abcam, Cambridge, Mass.), a rabbit polyclonal antibody raised against a synthetic peptide having amino acid sequence: ATLKDQLIYNLLKEEQTPQNKITVVGVGAVGMACAISILMKDLADELALV (SEQ ID NO: 1), corresponding to N terminal amino acids 2-51 of human LDH-5. Another antibody that blocks down-regulation of the immune system by LDH-5 is anti-Lactate Dehydrogenase antibody (ab85326 [AF14A11]; Abcam, Cambridge, Mass.), which is a mouse monoclonal to Lactate Dehydrogenase.

As described herein, five other antibodies were tested that are also specific for LDH-5, but they do not block the down-regulation of the immune system by LDH-5. Thus, it was shown that antibody binding to the amino terminus or to particular epitope(s) of LDH-5, had the surprising effects of blocking down-regulation of the immune system by LDH-5. Thus, the antibody binding to specified regions of LDH-5 led to a surprising effect of blocking down-regulation of the immune system by LDH-5. In some alternatives, an antibody or antibody binding fragment is provided, wherein the antibody is specific for LDH-5. In some alternatives, the antibody or antibody binding fragment is specific for an epitope on LDH-5. In some alternatives, the antibody or antibody binding fragment is specific for the amino acids within the amino terminus of LDH-5, wherein the amino acids are amino acid residues 1-166, 1-150, 1-125, 1-100, 1-75, 1-50, 1-25, 1-15, 1-10, or 1-5 or a range defined by any two numbers in between 1-166, including 1 and 166.

In some alternatives, a therapeutic antibody disclosed herein binds to LDH-5 or a variant thereof. Said antibody can be humanized in some alterantives.

In some alternatives, a therapeutic antibody disclosed herein binds to a receptor for LDH-5 or a variant thereof on an immune cell, wherein the antibody blocks binding of LDH-5 or a variant thereof to a receptor on an immune cell.

In some alternatives, the antibody or portion thereof comprises SEQ ID NO: 1. In some alternatives, this antibody is humanized.

### Other Agents and Methods.

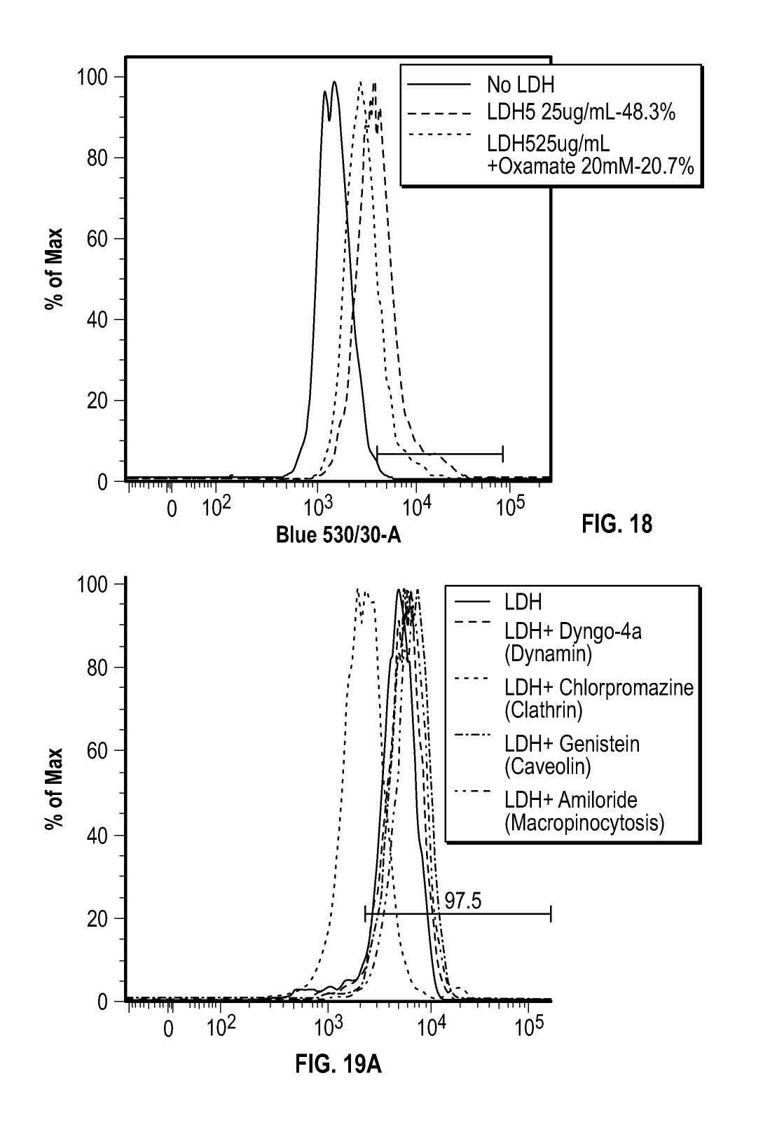

Some alternatives relate to a therapeutic dose of an inhibitor of LDH-5 activity. In some alternatives, the inhibitor is an enzymatic inhibitor (e.g., oxamate) (See FIG. 18). In other alternatives, the inhibitor is not an enzymatic inhibitor.

Some alternatives relate to modulation of expression of LDH-5, for example using RNAi and/or small molecules.

Some alternatives relate to a method of treating, inhibiting, or ameliorating cancer in a subject comprising administering to said subject a therapeutic dose of a nucleotide sequence encoding a peptide from the amino terminus of LDH-5. In some alternatives, the nucleotide sequence encodes a peptide having the amino acid sequence of SEQ ID NO: 1, SEQ ID NO: 2, SEQ ID NO: 3 or SEQ ID NO: 4.

“Combination therapy” as described herein, refers to a therapy that uses more than one medication or modality for a treatment. Combination therapy, for example can also refer to multiple therapies to treat a single disease, and often all the therapies are pharmaceutical. Combination therapy can also involve prescribing and administering separate drugs in which the dosage can also have more than one active ingredient. In some alternative, a combination therapy is provided. In some alternatives, the combination therapy comprises a therapeutic dose of an inhibitor of LDH-5 activity. In some alternatives, the combination therapy further comprises an inhibitor. In some alternatives, the inhibitor is not an enzymatic inhibitor. In some alternatives, the inhibitor is an enzymatic inhibitor. In some alternatives combination therapy comprises administering a therapeutic dose of an inhibitor of LDH-5 activity and an anti-MICB antibody, an anti-ULBP1 antibody and/or an anti-MICA antibody. These antibodies can be humanized in some embodiments.

“Chemotherapeutic drugs” are category of anti-cancer medicaments that can use, for example, chemical substances, such as anti-cancer drugs (chemotherapeutic agents) that can be given as part of a standardized chemotherapy regimen. Chemotherapeutic drugs can be given with a curative intent, or it can aim to prolong life or to reduce symptoms (palliative chemotherapy). Additional chemotherapies can also include hormonal therapy and targeted therapy, as it is one of the major categories of medical oncology (pharmacotherapy for cancer). These modalities are often used in conjunction with other cancer therapies, such as radiation therapy, surgery, and/or hyperthermia therapy.

However, traditional chemotherapeutic agents can be cytotoxic, for example, they can act by killing cells that divide rapidly, one of the main properties of most cancer cells. This means that chemotherapy also harms cells that divide rapidly under normal circumstances including cells in the bone marrow, digestive tract, and/or hair follicles.

Some newer anticancer drugs (for example, various monoclonal antibodies) are not indiscriminately cytotoxic, but rather target proteins that are abnormally expressed in cancer cells and that are essential for their growth. Such treatments are often referred to as targeted therapy (as distinct from classic chemotherapy) and are often used alongside traditional chemotherapeutic agents in antineoplastic treatment regimens. In some alternatives, the methods described herein can further comprise administering such targeted anti-cancer therapies.

Chemotherapy in which chemotherapeutic drugs are administered, can use one drug at a time (single-agent chemotherapy) or several drugs at once (combination chemotherapy or polychemotherapy). The combination of chemotherapy and radiotherapy is chemoradiotherapy. Chemotherapy using drugs that convert to cytotoxic activity only upon light exposure is called photochemotherapy or photodynamic therapy. In some alternatives of the methods described herein, the method can further comprise administering to a subject having cancer, photochemotherapy or photodynamic therapy.

Chemotherapuetic drugs can include but is not limited to antibody-drug conjugates (for example, an antibody attached to a drug by a linker), nanoparticles (for example a nanoparticle can be 1-1000 nanometer sized particle for promoting tumor selectivity and aid in delivering low-solubility drugs), electochemotherapy, alkylating agents, antimetabolites (for example, 5-fluorouracil (5-FU), 6-mercaptopurine (6-MP), Capecitabine (Xeloda®), Cladribine, Clofarabine, Cytarabine (Ara-C®), Floxuridine, Fludarabine, Gemcitabine (Gemzar®), Hydroxyurea, Methotrexate, Pemetrexed (Alimta®), Pentostatin, and Thioguanine), anti-tumor antibiotics, topoisomerase inhibitors, mitotic inhibitors, corticosteroids, DNA intercalating agents, and checkpoint inhibitors (for example checkpoint kinases CHK1, CHK2). In some alternatives of the methods described herein, the anti-LDH5 antibody or binding fragment thereof, which may be humanized, is administered in combination with one or more anti-cancer agents, such as any one or more of the foregoing compounds or therpies. In some alternatives, the one or more anti-cancer agent that is co-administered or administered within the same protocol for a patient as the anti-LDH5 antibody or binding fragment thereof, which may be humanized, comprises antibody-drug conjugates, nanoparticles, electochemotherapy, alkylating agents, antimetabolites, anti-tumor antibiotics, topoisomerase inhibitors, mitotic inhibitors, corticosteroids, DNA intercalating agents, and checkpoint inhibitors. In some alternatives, the antimetabolites comprises 5-fluorouracil (5-FU), 6-mercaptopurine (6-MP), Capecitabine (Xeloda®), Cladribine, Clofarabine, Cytarabine (Ara-C®), Floxuridine, Fludarabine, Gemcitabine (Gemzar®), Hydroxyurea, Methotrexate, Pemetrexed (Alimta®), Pentostatin, or Thioguanine.

“Checkpoint blockades” refers to a form of immunotherapy, meaning it aims to help the patient's own immune system fight cancer. It can use substances such as monoclonal antibodies, which can be designed to target extremely specific molecules on cell surfaces. For example, the antibodies unblock a reaction that stops the immune system's natural attack on invading cancer cells. In another example, a ligand-receptor interaction that has been investigated as a target for cancer treatment is the interaction between the transmembrane programmed cell death 1 protein (PDCD1, PD-1; also known as CD279) and its ligand, PD-1 ligand 1 (PD-L1, CD274). In normal physiology PD-L1 on the surface of a cell binds to PD1 on the surface of an immune cell, which inhibits the activity of the immune cell. It appears that upregulation of PD-L1 on the cancer cell surface can allow them to evade the host immune system by inhibiting T cells that might otherwise attack the tumor cell. Antibodies that bind to either PD-1 or PD-L1 and therefore block the interaction can allow the T-cells to attack the tumor. The LDH-5 antibody can be combined in some alternatives, with checkpoint blockade immunotherapeutics. In some alternatives, the checkpoint blockade therapeutics comprises PD-1 antibodies. In some alternatives, the checkpoint blockade therapeutics comprises PD-L1. In some alternatives, the one or more anti-cancer agent that is co-administered or administered within the same protocol for a patient as the anti-LDH5 antibody or binding fragment thereof, which may be humanized, comprises one or more of such checkpoint blockades.

“Small molecule inhibitors” as described herein refers to small inhibitors that can target proteins of interest. The proteins can be proteins that are secreted by tumor cells or proteins secreted during cellular stress. The protein can also be LDH-5 in which an inhibitor is sought in order to inhibit the activity of LDH-5. Small molecule inhibitors can include but are not limited to kinase inhibitors, inhibitors of Bcl-2 family proteins for cancer therapy, MCl-1 inhibitors, and tyrosine kinase inhibitors. Tyrosine kinase inhibitors can include but are not limited to Imatinib mesylate (approved for chronic myelogenous leukemia, gastrointestinal stromal tumor and some other types of cancer), Gefitinib (Iressa, also known as ZD1839); targets the epidermal growth factor receptor (EGFR) tyrosine kinase) Erlotinib (marketed as Tarceva), Sorafenib, Sunitinib (Sutent), Dasatinib (Srycel), Lapatinib (Tykerb), Nilotinib (Tasigna), Bortezomib (Velcade), Janus kinase inhibitors, ALK inhibitors, crizotinib Bcl-2 inhibitors, obatoclax, navitoclax, gossypol, PARP inhibitors, Iniparib, Olaparib, PI3K inhibitors, perifosine, Apatinib, VEGF Receptor 2 inhibitors, AN-152, Braf inhibitors, vemurafenib, dabrafenib, LGX818, MEK inhibitors, trametinib, MEK162, CDK inhibitors, PD-0332991, Hsp90 inhibitors, and salinomycin. In some alternatives, the one or more anti-cancer agent that is co-administered or administered within the same protocol for a patient as the anti-LDH5 antibody or binding fragment thereof, which may be humanized, comprises one or more of such small molecule inhibitors. In some alternatives, the small molecule inhibitors that are used comprise kinase inhibitors. Small molecule inhibitors can also include small molecules that can bind LDH-5 and inhibit activity. In some alternatives, the small molecule inhibitors comprise inhibitors of Bcl-2 family proteins. In some alternatives, the small molecule inhibitors comprise MCl-1 inhibitors. In some alternatives, the small molecule inhibitors comprise tyrosine kinase inhibitors. In some alternatives, the small molecule inhibitor is Imatinib. In some alternatives, the small molecule inhibitor is mesylate. In some alternatives, the small molecule inhibitor is Gefitinib. In some alternatives, the small molecule inhibitor is Erlotinib. In some alternatives, the small molecule inhibitor is Sorafenib. In some alternatives, the small molecule inhibitor is Sunitinib (Sutent). In some alternatives, the small molecule inhibitor is Dasatinib. In some alternatives, the small molecule inhibitor is Lapatinib (Tykerb). In some alternatives, the small molecule inhibitor is Nilotinib (Tasigna). In some alternatives, the small molecule inhibitor is Bortezomib (Velcade). In some alternatives, the small molecule inhibitors are Janus kinase inhibitors. In some alternatives, the small molecule inhibitor is an ALK inhibitor. In some alternatives, the small molecule inhibitor is crizotinib. In some alternatives, the small molecule inhibitors are Bcl-2 inhibitors. In some alternatives, the small molecule inhibitor is obatoclax. In some alternatives, the small molecule inhibitor is navitoclax. In some alternatives, the small molecule inhibitor is gossypol. In some alternatives, the small molecule inhibitors are PARP inhibitors. In some alternatives, the small molecule inhibitor is Iniparib. In some alternatives, the small molecule inhibitor is Olaparib. In some alternatives, the small molecule inhibitor are PI3K inhibitors. In some alternatives, the small molecule inhibitor is perifosine. In some alternatives, the small molecule inhibitor is Apatinib. In some alternatives, the small molecule inhibitors are tyrosine VEGF Receptor 2 inhibitors. In some alternatives, the small molecule inhibitor is AN-152. In some alternatives, the small molecule inhibitors are Braf inhibitors. In some alternatives, the small molecule inhibitor is vemurafenib. In some alternatives, the small molecule inhibitor is dabrafenib. In some alternatives, the small molecule inhibitor is LGX818. In some alternatives, the small molecule inhibitors are MEK inhibitors. In some alternatives, the small molecule inhibitor is trametinib. In some alternatives, the small molecule inhibitor is MEK162. In some alternatives, the small molecule inhibitors are CDK inhibitors. In some alternatives, the small molecule inhibitor is PD-0332991. In some alternatives, the small molecule inhibitors are Hsp90 inhibitors. In some alternatives, the small molecule inhibitor is salinomycin. Again, in some alternatives, the one or more anti-cancer agent that is co-administered or administered within the same protocol for a patient as the anti-LDH5 antibody or binding fragment thereof, which may be humanized, comprises any one or more of the aforementioned small molecule inhibitors.

“Adoptive cellular therapy” as described herein refers to the transfer of cells, most commonly immune-derived cells, back into the same patient or into a new recipient host with the goal of transferring the immunologic functionality and characteristics into the new host. In an example, T cell adoptive therapy can comprise the addition of chimeric antigen receptors, or CARs, to redirect the specificity of cytotoxic and helper T cells. T cell adoptive therapy can also include administering T-cells with engineered T cell receptors (TCRs) for specificity to specific epitopes on a cell of interest. The LDH-5 antibody or binding fragment thereof, which can be humanized, can be combined in some alternatives, with adoptive cellular therapy. In some alternatives, adoptive cellular therapy comprises administering T cells comprising chimeric antigen receptors.

In some alternatives, a method of inhibiting, ameliorating, or treating cancer in a subject is provided, wherein the method comprises selecting a subject to receive a binding agent specific for Lactate dehydrogenase 5 (LDH-5), or selecting a subject to receive an anti-cancer therapy, providing to said subject an amount of a binding agent, such as a fragment comprising a CDR domain or a Fab fragment, that is specific for LDH-5, preferably a binding agent that is specific for amino acid residues 1-241, 1-200, 1-175, 1-150, 1-125, 1-100, 1-75, 1-50, 1-25, 1-15, 1-10, or 1-5 or a range defined by any two numbers in between 1-241, including 1 and 241 of SEQ ID NOs 1, 2, 3 or 4, and optionally, observing, analyzing, monitoring, or measuring an inhibition, amelioration, or treatment of cancer in said subject. In some alternatives, a method of treating, inhibiting, or ameliorating cancer in a subject is provided, wherein the method comprises detecting LDH-5, or LDH-5 activity in a biological sample obtained from said subject, and administering to said subject a therapeutic dose of an anti-LDH-5 antibody or binding fragment thereof raised against the amino terminus of said LDH-5 or a variant thereof such as, amino acid residues 1-241, 1-200, 1-175, 1-150, 1-125, 1-100, 1-75, 1-50, 1-25, 1-15, 1-10, or 1-5 or a range defined by any two numbers in between 1-241, including 1 and 241 of SEQ ID NOs 1, 2, 3 or 4. In some alternatives, a method of treating, inhibiting, or ameliorating cancer in a subject is provided, wherein the method comprises administering to said subject a therapeutic dose of a nucleotide sequence encoding a peptide from the amino terminus of LDH-5. In some alternatives, a method of treating, inhibiting, or ameliorating cancer in a subject is provided, wherein the method comprises detecting LDH-5, or LDH-5 activity in a biological sample obtained from said subject, administering to said subject a therapeutic dose of an anti-LDH-5 antibody or binding fragment thereof raised against the amino terminus of said LDH-5 or a variant thereof such as, amino acid residues 1-241, 1-200, 1-175, 1-150, 1-125, 1-100, 1-75, 1-50, 1-25, 1-15, 1-10, or 1-5 or a range defined by any two numbers in between 1-241, including 1 and 241 of SEQ ID NOs 1, 2, 3 or 4 and administering one or more other anti-cancer agents. In some alternatives, a method of stimulating an immune system in a subject with cancer is provided, wherein the method comprises selecting a subject to receive a binding agent specific for Lactate dehydrogenase 5 (LDH-5), or selecting a subject to receive an anti-cancer therapy, providing to said subject an amount of a binding agent, such as a fragment comprising a CDR domain or a Fab fragment, that is specific for LDH-5, preferably a binding agent that is specific for amino acid residues 1-241, 1-200, 1-175, 1-150, 1-125, 1-100, 1-75, 1-50, 1-25, 1-15, 1-10, or 1-5 or a range defined by any two numbers in between 1-241, including 1 and 241 of SEQ ID NOs 1, 2, 3 or 4, preferably said binding agent binds to LDH-5 and optionally, observing, analyzing, monitoring, or measuring an inhibition, amelioration, or treatment of cancer in said subject. In some alternatives of the methods provided herein, the methods further comprise administering in combination with the binding agent, such as a fragment comprising a CDR domain or a Fab fragment, that is specific for LDH-5, immunotherapy treatments, wherein the immunotherapy treatments modulate immune cells, wherein the treatments comprise at least one of checkpoint blockades, small molecule inhibitors, and/or adoptive cellular therapies. In some alternatives, of the methods provided herein, the method further comprises administering in combination with the therapeutic dose of an anti-LDH-5 antibody or binding fragment thereof, immunotherapy treatments, wherein the immunotherapy treatments modulate immune cells, wherein the treatments comprise at least one of checkpoint blockades, small molecule inhibitors, and/or adoptive cellular therapies. In some alternatives, of the methods described herein the method further comprises administering in combination with a therapeutic dose of a nucleotide sequence encoding a peptide from the amino terminus of LDH-5, immunotherapy treatments, wherein the immunotherapy treatments modulate immune cells, wherein the treatments comprise at least one of checkpoint blockades, small molecule inhibitors, and/or adoptive cellular therapies. In some alternatives, the checkpoint blockade therapeutics comprises PD-1 antibodies. In some alternatives, the checkpoint blockade therapeutics comprises PD-L1. The LDH-5 antibody can be combined in some alternatives, with adoptive cellular therapy. In some alternatives, adoptive cellular therapy comprises administering T cells comprising chimeric antigen receptors. In some alternatives, the adoptive cellular therapies comprise administering T-cells comprising chimeric antigen receptors. In some alternatives, the chimeric antigen receptors target epitopes on tumors. In some alternatives, the small molecule inhibitors are kinase inhibitors. In some alternatives, the small molecules are Chk1,2 inhibitors. In some alternatives, the small molecule inhibitors comprise inhibitors of Bcl-2 family proteins. In some alternatives, the small molecule inhibitors comprise MCl-1 inhibitors. In some alternatives, the small molecule inhibitors comprises tyrosine kinase inhibitors. In some alternatives, the small molecule inhibitor is Imatinib. In some alternatives, the small molecule inhibitor is mesylate. In some alternatives, the small molecule inhibitor is Gefitinib. In some alternatives, the small molecule inhibitor is Erlotinib. In some alternatives, the small molecule inhibitor is Sorafenib. In some alternatives, the small molecule inhibitor is Sunitinib (Sutent). In some alternatives, the small molecule inhibitor is Dasatinib. In some alternatives, the small molecule inhibitor is Lapatinib (Tykerb). In some alternatives, the small molecule inhibitor is Nilotinib (Tasigna). In some alternatives, the small molecule inhibitor is Bortezomib (Velcade). In some alternatives, the small molecule inhibitors are Janus kinase inhibitors. In some alternatives, the small molecule inhibitor is an ALK inhibitor. In some alternatives, the small molecule inhibitor is crizotinib. In some alternatives, the small molecule inhibitors are Bcl-2 inhibitors. In some alternatives, the small molecule inhibitor is obatoclax. In some alternatives, the small molecule inhibitor is navitoclax. In some alternatives, the small molecule inhibitor is gossypol. In some alternatives, the small molecule inhibitors are PARP inhibitors. In some alternatives, the small molecule inhibitor is Iniparib. In some alternatives, the small molecule inhibitor is Olaparib. In some alternatives, the small molecule inhibitor are PI3K inhibitors. In some alternatives, the small molecule inhibitor is perifosine. In some alternatives, the small molecule inhibitor is Apatinib. In some alternatives, the small molecule inhibitors are tyrosine VEGF Receptor 2 inhibitors. In some alternatives, the small molecule inhibitor is AN-152. In some alternatives, the small molecule inhibitors are Braf inhibitors. In some alternatives, the small molecule inhibitor is vemurafenib. In some alternatives, the small molecule inhibitor is dabrafenib. In some alternatives, the small molecule inhibitor is LGX818. In some alternatives, the small molecule inhibitors are MEK inhibitors. In some alternatives, the small molecule inhibitor is trametinib. In some alternatives, the small molecule inhibitor is MEK162. In some alternatives, the small molecule inhibitors are CDK inhibitors. In some alternatives, the small molecule inhibitor is PD-0332991. In some alternatives, the small molecule inhibitors are Hsp90 inhibitors. In some alternatives, the small molecule inhibitor is salinomycin. Again, in some alternatives, the one or more anti-cancer agent that is co-administered or administered within the same protocol for a patient as the anti-LDH5 antibody or binding fragment thereof, which may be humanized, comprises any one or more of the aforementioned inhibitors.

Small inhibitors can also include serine/threonine kinase inhibitors. Without being limiting examples are Temsirolimus (Torisel), Everolimus (Afinitor), Vemurafenib (Zelboraf), Trametinib (Mekinist) and Dabrafenib (Tafinlar). In some alternatives, the small molecule inhibitor is Temsirolimus (Torisel). In some alternatives, the small molecule inhibitor is Everolimus (Afinitor). In some alternatives, the small molecule inhibitor is Vemurafenib (Zelboraf). In some alternatives, the small molecule inhibitor is Trametinib (Mekinist). In some alternatives, the small molecule inhibitor is Dabrafenib (Tafinlar). Again, in some alternatives, the one or more anti-cancer agent that is co-administered or administered within the same protocol for a patient as the anti-LDH5 antibody or binding fragment thereof, which may be humanized, comprises any one or more of the aforementioned inhibitors.

In some alternatives of the methods provided herein, particularly in combination with treatments predicated on the activation of immune cells, the LDH-5 antibody or binding agent can be administered with checkpoint blockades, small molecule inhibitors, and adoptive cellular therapies, such as anti-CTLA-4 antibodies, anti-PD1 antibodies, anti-PD-L1 antibodies, Chk1,2 inhibitors, CAR's, and TCR's. In some alternatives, the small molecule inhibitor is Temsirolimus (Torisel). In some alternatives, the small molecule inhibitor is Everolimus (Afinitor). In some alternatives, the small molecule inhibitor is Vemurafenib (Zelboraf). In some alternatives, the small molecule inhibitor is Trametinib (Mekinist). In some alternatives, the small molecule inhibitor is Dabrafenib (Tafinlar). In some alternatives, the small molecule inhibitors are kinase inhibitors. In some alternatives, the small molecules are Chk1,2 inhibitors. In some alternatives, the small molecule inhibitors comprise inhibitors of Bcl-2 family proteins. In some alternatives, the small molecule inhibitors comprise MCl-1 inhibitors. In some alternatives, the small molecule inhibitors comprise tyrosine kinase inhibitors. In some alternatives, the small molecule inhibitor is Imatinib. In some alternatives, the small molecule inhibitor is mesylate. In some alternatives, the small molecule inhibitor is Gefitinib. In some alternatives, the small molecule inhibitor is Erlotinib. In some alternatives, the small molecule inhibitor is Sorafenib. In some alternatives, the small molecule inhibitor is Sunitinib (Sutent). In some alternatives, the small molecule inhibitor is Dasatinib. In some alternatives, the small molecule inhibitor is Lapatinib (Tykerb). In some alternatives, the small molecule inhibitor is Nilotinib (Tasigna). In some alternatives, the small molecule inhibitor is Bortezomib (Velcade). In some alternatives, the small molecule inhibitors are Janus kinase inhibitors. In some alternatives, the small molecule inhibitor is an ALK inhibitor. In some alternatives, the small molecule inhibitor is crizotinib. In some alternatives, the small molecule inhibitors are Bcl-2 inhibitors. In some alternatives, the small molecule inhibitor is obatoclax. In some alternatives, the small molecule inhibitor is navitoclax. In some alternatives, the small molecule inhibitor is gossypol. In some alternatives, the small molecule inhibitors are PARP inhibitors. In some alternatives, the small molecule inhibitor is Iniparib. In some alternatives, the small molecule inhibitor is Olaparib. In some alternatives, the small molecule inhibitors are PI3K inhibitors. In some alternatives, the small molecule inhibitor is perifosine. In some alternatives, the small molecule inhibitor is Apatinib. In some alternatives, the small molecule inhibitors are tyrosine VEGF Receptor 2 inhibitors. In some alternatives, the small molecule inhibitor is AN-152. In some alternatives, the small molecule inhibitors are Braf inhibitors. In some alternatives, the small molecule inhibitor is vemurafenib. In some alternatives, the small molecule inhibitor is dabrafenib. In some alternatives, the small molecule inhibitor is LGX818. In some alternatives, the small molecule inhibitors are MEK inhibitors. In some alternatives, the small molecule inhibitor is trametinib. In some alternatives, the small molecule inhibitor is MEK162. In some alternatives, the small molecule inhibitors are CDK inhibitors. In some alternatives, the small molecule inhibitor is PD-0332991. In some alternatives, the small molecule inhibitors are Hsp90 inhibitors. In some alternatives, the small molecule inhibitor is salinomycin. Again, in some alternatives, the one or more anti-cancer agent that is co-administered or administered within the same protocol for a patient as the anti-LDH5 antibody or binding fragment thereof, which may be humanized, comprises any one or more of the aforementioned inhibitors. In some alternatives, adoptive cellular therapies comprises administering T cells comprising chimeric antigen receptors (CARs). In some alternatives, the CARs are engineered to bind to an epitope on a tumor cell.

### LDH-5 and Variants.

LDH-5 (L-lactate dehydrogenase A chain isoform 5; Genbank Accession No. NP_001158888) is expressed in human and has the amino acid sequence:

(SEQ ID NO: 2)
MATLKDQLIYNLLKEEQTPQNKITVVGVGAVGMACAISILMKDLADELAL
VDVIEDKLKGEMMDLQHGSLFLRTPKIVSGKDYNVTANSKLVIITAGARQ
QEGESRLNLVQRNVNIFKFIIPNVVKYSPNCKLLIVSNPVDILTYVAWKI
SGFPKNRVIGSGCNLDSARFRYLMGERLGVHPLSCHGWVLGEHGDSSVPV
WSGMNVAGVSLKTLHPDLGTDKDKEQWKEVHKQVVERVFTE.

A variant of LDH-5, as disclosed herein, can share 70%, 75%, 80%, 85%, 90%, 95%, 96%, 97%, 98%, or 99% sequence identity with SEQ ID NO: 1, 2, 3 or 4 or any other percent sequence identity within a range defined by any two of the aforementioned percentages. The methods disclosed herein relate to LDH isozymes that are cross-reactive with the LDH-5 receptor and/or are internalized by immune cells.

In some alternatives, binding agents block LDH-5 receptor binding, incorporation, internalization and/or endocytosis to immune cells or tumor cells. Some binding agents are cross reactive with other LDH types, especially those containing LDH 2-5, especially LDH-A.

### Immune Response.

The immune response has an important role in cancer, for identification and elimination of tumors. For example, transformed cells of tumors can express antigens that are not found on normal cells. To the immune system, these antigens appear foreign, and their presence causes immune cells to attack the transformed tumor cells. The antigens expressed by tumors have several sources, for example, some are derived from oncogenic viruses like human papillomavirus, which can cause cervical cancer, while in other examples, the organism's own proteins that occur at low levels in normal cells can reach high levels in tumor cells. One example is an enzyme called tyrosinase that, when expressed at high levels, transforms certain skin cells (e.g. melanocytes) into tumors called melanomas. A third possible source of tumor antigens are proteins normally important for regulating cell growth and survival, that commonly mutate into cancer inducing molecules called oncogenes.

The main response of the immune system to tumors is to destroy the abnormal cells using killer T cells, sometimes with the assistance of helper T cells. Tumor antigens are presented on MHC class I molecules in a similar way to viral antigens. This allows killer T cells to recognize the tumor cell as abnormal. NK cells also kill tumorous cells in a similar way, especially if the tumor cells have fewer MHC class I molecules on their surface than normal; this is a common phenomenon with tumors. Sometimes antibodies are generated against tumor cells allowing for their destruction by the complement system.

In some examples, tumors can evade the immune system and go on to become cancers. Tumor cells often have a reduced number of MHC class I molecules on their surface, thus avoiding detection by killer T cells. Some tumor cells also release products that inhibit the immune response; for example, by secreting the cytokine TGF-β, which suppresses the activity of macrophages and lymphocytes. In addition, immunological tolerance may develop against tumor antigens, so the immune system no longer attacks the tumor cells.

Paradoxically, macrophages can promote tumor growth when tumor cells send out cytokines that attract macrophages, which then generate cytokines and growth factors that nurture tumor development. In addition, a combination of hypoxia in the tumor and a cytokine produced by macrophages induces tumor cells to decrease production of a protein that blocks metastasis and thereby assists spread of cancer cells.

Stimulating the immune system in an individual with cancer can help to decrease, and inhibit tumor growth in the individual. Immune system stimulation can also increase the response of the immune system to destroy the abnormal cells using killer T cells, sometimes with the assistance of helper T cells. Stimulation of the immune system can lead to NK T cells to produce large quantities of interferon gamma, IL-4, granulocyte macrophage colony stimulating factor, as well as multiple cytokines and chemokines, such as for example IL-2, Interleukin 13, Interleukin-17, Interleukin-21, and TNF-alpha. Immune response stimulation would lead to a strong response in the identification and elimination of tumors. In some alternatives, a method of stimulating the immune system is provided, wherein the method comprises selecting a subject to receive a binding agent specific for Lactate dehydrogenase 5 (LDH-5), or selecting a subject to receive an anti-cancer therapy, providing to said subject an amount of a binding agent, such as a fragment comprising a CDR domain or a Fab fragment, that is specific for LDH-5, preferably a binding agent that is specific for amino acid residues 1-241, 1-200, 1-175, 1-150, 1-125, 1-100, 1-75, 1-50, 1-25, 1-15, 1-10, or 1-5 or a range defined by any two numbers in between 1-241, including 1 and 241 of SEQ ID NOs 1, 2, 3 or 4, preferably said binding agent binds to LDH-5, and optionally, observing, analyzing, monitoring, or measuring an inhibition, amelioration, or treatment of cancer in said subject. In some alternatives, the binding agent is an anti-LDH-5 antibody or binding fragment thereof, such as a fragment comprising a CDR domain or a Fab fragment, preferably a monoclonal antibody or binding fragment thereof, such as a fragment comprising a CDR domain or a Fab fragment. In some alternatives, the anti-LDH-5 antibody or binding fragment thereof is directed to the amino terminus of LDH-5, such as amino acid residues 1-166, 1-150, 1-125, 1-100, 1-75, 1-50, 1-25, 1-15, 1-10, or 1-5 or a range defined by any two numbers in between 1-166, including 1 and 166 of SEQ ID NOs 1, 2, 3 or 4. In some alternatives, the antibody is directed against a peptide having the amino acid sequence of SEQ ID NO: 1, 2, 3 or 4. In some alternatives, the antibody is Ab84716. In some alternatives, the antibody is a monoclonal antibody. In some alternatives, the monoclonal antibody is Ab85326, also named AF14A11. In some alternatives, the cancer is in the brain, endometrium, colon, blood, lung or epithelium/mouth. In some alternatives, the lung cancer is non-small cell lung carcinoma (NSCLC). In some alternatives, the cancer of the epithelium/mouth is a squamous cell carcinoma. In some alternatives, the cancer is a glioma. In some alternatives, the glioma is a glioblastoma. In some alternatives, the agent inhibits LDH-5 binding and/or incorporation and/or internalization and/or endocytosis to a lymphocyte, an NK Cell, an NKG2D-expressing cell and/or a monocyte.

### Patient Samples.

Tumor tissue and blood were collected from patients with glioblastoma multiforme (GBM) during surgical resection. Blood also was collected from patients free of malignant glioma tumor burden during procedures such as meningioma resection. Meningioma-resected patients were used as negative controls for two reasons. First, because blood was collected from both patient populations at the time of surgery, all patients had been subjected to a standard algorithm of preoperative management including, but not limited to, antibiotic administration, a bolus of dexamethasone, and mannitol, as well as other medications including anticonvulsant drugs. Additionally, healthy controls are unavailable because, the patients must undergo intracranial surgery to obtain tumor-infiltrating lymphocytes. All patients gave informed consent for sample acquisition under the University of California San Francisco (UCSF) Internal Review Board (IRB)-approved Brain Tumor Research Center protocol CHR #10-01271. Longitudinal GBM patient samples were obtained from patients on a Phase I/II clinical trial for autologous heat shock protein vaccination that was approved by the UCSF IRB and was done in accordance with the Declaration of Helsinki and guidelines for Good Clinical Practice. The clinical trial is registered at clinicaltrials.gov. Peripheral blood mononuclear cells (PBMCs) from healthy donors were obtained from leukocyte reduction Pall filters (Blood Centers of the Pacific) and processed, as described below.

### Tumor-Infiltrating and Circulating Mononuclear Cell Isolation.

PBMCs were isolated from whole blood by a Ficoll-Paque Plus density gradient centrifugation (GE HealthCare). Tumor-infiltrating lymphocytes were isolated from tissue samples by mincing tumor samples and treating with 1 mg/mL collagenase D (Sigma-Aldrich) in PBS for 30 min followed by Percoll density gradient centrifugation (Sigma-Aldrich).

### Cell Selections.

Monocytes and natural killer (NK) cells were isolated using NK cell- or monocyte-positive selection kits according to the manufacturer's instructions (StemCell Technologies, Inc.).

### Cell Lines.

293T and the glioma cell lines U87, SF767, and U251 were obtained from the UCSF Brain Tumor Research Center. Cell lines were cultured in DMEM H-21 with 10% (vol/vol) FBS and 1% penicillin and streptomycin.

NKG2D Ligand Induction on Freshly Isolated Monocytes from Healthy Donors.

U87 tumor-conditioned medium was generated by culturing U87 cells in complete RPMI medium (RPMI-1640, 25 mM Hepes, 2.0 g/L NaHCO3 supplemented with 2% (vol/vol) FBS, 1% penicillin-streptomycin, 1 mM sodium pyruvate, and 10 mM nonessential amino acids) for 48 h when tumor cells were confluent. The U87-conditioned medium was diluted 1:1 with fresh complete RPMI medium to replenish nutrients and was used in subsequent assays for culturing with freshly isolated peripheral blood monocytes for 18-72 h.

### Purification and Identification of Natural Killer Group 2, Member D Ligand-Inducing Factor.

The Natural killer group 2, member D (NKG2D) ligand-inducing factor present in U87-conditioned medium was identified by chromatographic separations and assaying fractions for their ability to induce transcription of MICB and ULBP-1 when cultured with monocytes isolated from healthy donors. Briefly, 12 L of U87 medium supernatant was concentrated 10-fold using Amicon Ultra-15 protein concentrators with a 10-kDA restriction (Millipore). Proteins then were precipitated with 60-100% saturated ammonium sulfate. Active fractions were purified further on a Phenyl Sepharose column (GE Healthcare) by loading in 1 M ammonium sulfate and eluting with a 100 mM Na2-HSO4-1 M Na2 HSO4 gradient. The NKG2D ligand-inducing activity eluted in the range of 72-20 ms/cm and was purified further on a Superdex-200 column (GE Healthcare) and eluted in the range of 40-70 kDa. The activity-containing fraction was run over a MonoS column (GE Healthcare) using a binding buffer of 25 mM KCl, 50 mM sodium acetate, pH 5.0, and the elution buffer, 1 M KCl, 50 mM sodium acetate, pH 5.0. The activity eluted between 27 and 42 ms/cm. The purified activity retaining fraction was analyzed in-gel and in-solution by MS-MS by the UCSF Mass Spectrometry Facility and the National Bio-Organic Biomedical Mass Spectrometry Resource Center.

### Flow Cytometry.

Cells were stained for 25 min on ice with monoclonal antibodies and washed in PBS with 2% (wt/vol) BSA. FITC-conjugated anti-ULBP-1, phycoerythrin (PE)-conjugated anti-MICA/B, PE-Cy7-conjugated anti-CD56, FITC-conjugated anti-CD3, allophycocyanin (APC)-conjugated anti-NKG2D, PECy7-conjugated anti-CD14, APC-conjugated anti-HLA-DR, Pacific Blue-conjugated anti-CD11b, APC-conjugated anti-IFN-γ, FITC-conjugated anti-granzyme B, PE-conjugated anti-perforin, and APC- or PE-conjugated anti-CD107a were purchased from BD Pharmingen, R&D Systems, Inc., or eBioscience. Samples were analyzed using a BD FACSCalibur with Cell Quest Pro software, and data were analyzed using FlowJo software (TreeStar). Fluorochrome-conjugated isotype-matched control Igs were used to detect specific staining of cell populations.

### Quantitative PCR.

Whole-cell mRNA was extracted by using the RNeasy Mini Kit (Qiagen) and then was converted to cDNA using random hexamer priming and SuperScript III reverse transcriptase (100-250 ng RNA per reaction) (Invitrogen). PCR amplification was performed with SYBR Green master mix (5-10 ng cDNA per reaction) (Applied Biosystems) using an iQ5 Real-Time PCR thermal cycler (Bio-Rad). Reactions were performed in duplicate. Cycle threshold (Ct) values of NKG2D ligands were normalized to expression levels of hypoxanthine phosphoribosyltransferase and displayed as relative expression units. Primer sequences are listed in Table 1.

TABLE 1
Primers used in Study

| Gene | Forward primer | Reverse primer |
|---|---|---|
| ULBP-1 | TGCAGGCCAGGATGTCTTGT | CATCCCTGTTCTTCTCCCACTTC |
| | (SEQ ID NO: 5) | (SEQ ID NO: 6) |
| ULBP-2 | CAGAGCAACTGCGTGACATT | GGCCACAACCTTGTCATTCT |
| | (SEQ ID NO: 7) | (SEQ ID NO: 8) |
| ULBP-3 | GGATTTCACACCCAGTGGAC | GCCTCTTCTTCCTGTGCATC |
| | (SEQ ID NO: 9) | (SEQ ID NO: 10) |
| ULBP-4 | GGCTCAGGGAATTCTTAGGG | CATTTTGCCACCAGACACAG |
| | (SEQ ID NO: 11) | (SEQ ID NO: 12) |
| ULBP-5 | CAAGACAGTCACACCCGTCA | AAGCCATCCTGTGCAGTCTC |
| | (SEQ ID NO: 13) | (SEQ ID NO: 14) |
| ULBP-6 | TGACATCACCGTCATCCCTA | TGCTCACAAGACATCCTTGC |
| | (SEQ ID NO: 15) | (SEQ ID NO: 16) |
| MICA | ACAATGCCCCAGTCCTCCAGA | ATTTTAGATATCGCCGTAGTTCT |
| | (SEQ ID NO: 17) | (SEQ ID NO: 18) |
| MICB | TGAGCCCCACAGTCTTCGTTA | CCTGCGTTTCTGCCTGTCATA |
| | (SEQ ID NO: 19) | (SEQ ID NO: 20) |

### NK Cell Functional Assays.

Freshly isolated NK cells were used in assays immediately after selection or were activated overnight with 500 U/mL recombinant IL-2 (National Cancer Institute Biological Resources Branch). Unless otherwise indicated, NK cells were cultured at a 1:1 ratio with monocytes or glioma tumor cells in the presence of PE-conjugated anti-CD107a for 3-18 h. Intracellular protein expression of cytokines and cytolytic granules was measured by adding brefeldin A to cultures 2-6 h before the end of the assay, and subsequently cells were fixed using 1% paraformaldehyde in PBS and then were stained for intracellular IFN-γ, perforin, and/or granzyme B. Target cell apoptosis was measured by staining with antibodies, listed above to distinguish tumor cells, NK cells, and monocytes. Tumor cells were defined as CD45−, MHC class I+ cells; monocytes were defined as CD45+CD14+ MHC class II+ cells; and NK cells were defined as CD45+, CD3−, CD56+ cells. Cells were stained for extracellular annexin V using Annexin Staining buffer (BD Pharmingen) to detect apoptotic cells.

### Lactate Dehydrogenase Assays.

Induction of NKG2D ligands was evaluated by using purified native lactate dehydrogenase (LDH) isoenzyme 5 (MyBioSource or Abcam) at the indicated concentrations, expressed in enzymatically active units per liter. Monocytes were cultured for 24 h in RPMI complete medium.

### LDH Inhibition.

U87 glioma cells were washed and cultured for 24 h in the presence of 20 mM sodium oxamate (Sigma-Aldrich). Sodium oxamate then was dialyzed from the supernatant, and supernatant was added to monocytes from healthy donors for 24 h. Cells were analyzed for induction of MHC class I polypeptide-related sequence A (MICA), MHC class I polypeptide-related sequence B (MICB), and UL16-binding protein 1 (ULBP1) mRNA.

### LDH Expression.

LDH-A and LDH-B cDNA were amplified from RNA isolated from U87 cells using the following primer sequences: LDH-A 5′: CGGCCAGAATTCCGCCACCATGGCAACTCTAAAGGATCAGCTGA (5′ Eco RI site) (SEQ ID NO: 21); LDH-A 3′: CGCCGACTCGAGTAA TAAAATTGCAGCTCCTTTTGGATC (3′ Xho I site) (SEQ ID NO: 22); LDH-B 5′: CGCCGAGGTACCCGCCACCATGGCAACTCTTAAGGAAAAACTC (5′ Kpn I site) (SEQ ID NO: 23); LDHB 3′: CGCCGAGAATTCTAATCACAGGTCTTTTAGGTCCTTCTG (3′ Eco RI site) (SEQ ID NO: 24). Restriction-digested cDNA was cloned into pcDNA3.1+ vector, and plasmids were amplified in Top10 competent Escherichia coli. Sequence analysis identified positive clones, and plasmid DNA was purified using a Qiagen MaxiPrep kit. One microgram of DNA was transiently transfected into 293T cells using Lipofectamine 2000. Seventy-two hours later, supernatant was analyzed to confirm LDH activity and assayed for NKG2D ligand induction on monocytes isolated from healthy donors.

### LDH Activity Assay.

Supernatants and patient sera were analyzed for LDH enzymatic activity using a commercially available colorimetric assay according to the manufacturer's instructions (Sigma-Aldrich). Standard curves were generated using purified native LDH5.

Acquisition of PBMCs from Patients with Other Solid Tumors.

Five milliliters of peripheral blood was collected from (i) breast cancer patients using the IRB-approved protocol UCSF CHR #H8409-27022-05 in collaboration with Michael Campbell; (ii) prostate cancer patients in collaboration with Larry Fong and John Kurhanewicz (IRB approval UCSF CHR #H7579-03002); and (iii) patients with hepatocellular carcinoma in collaboration with Stewart Cooper from the Ibrahim El-Hefni Liver Biorepository at California Pacific Medical Center [California Pacific Medical Center (CPMC) IRB approval CPMC #25.117 and 27.102].

### Statistical Analysis.

Data shown in all figures were collected from at least two independent experiments performed in triplicate. Representative raw experimental data are shown to clarify gating strategy and are summarized in Excel format. Determinations of statistical significance were made based on integrated experimental data. Statistical significance was determined by using a two-tailed Student t test with P<0.05, as determined by analysis in Prism GraphPad software or Excel. Error bars represent ±SD. Quantitative PCR was analyzed using the Pfaffl method, and statistical significance was determined using a nonparametric Wilcoxon two-group test.

### Additional Alternatives.

In some alternatives, a method of inhibiting, ameliorating, or treating cancer in a subject is provided, wherein the method comprises selecting a subject to receive a binding agent specific for Lactate dehydrogenase 5 (LDH-5), or selecting a subject to receive an anti-cancer therapy, providing to said subject an amount of a binding agent, such as a fragment comprising a CDR domain or a Fab fragment, that is specific for LDH-5, preferably a binding agent that is specific for amino acid residues 1-241, 1-200, 1-175, 1-150, 1-125, 1-100, 1-75, 1-50, 1-25, 1-15, 1-10, or 1-5 or a range defined by any two numbers in between 1-241, including 1 and 241 of SEQ ID NOs 1, 2, 3 or 4 and optionally, observing, analyzing, monitoring, or measuring an inhibition, amelioration, or treatment of cancer in said subject. In some alternatives, the binding agent is an anti-LDH-5 antibody or binding fragment thereof, such as a fragment comprising a CDR domain or a Fab fragment, preferably a monoclonal antibody or binding fragment thereof, such as a fragment comprising a CDR domain or a Fab fragment, any of which may be humanized. In some alternatives, the anti-LDH-5 antibody or binding fragment thereof is directed to the amino terminus of LDH-5, such as amino acid residues 1-166, 1-150, 1-125, 1-100, 1-75, 1-50, 1-25, 1-15, 1-10, or 1-5 or a range defined by any two numbers in between 1-166, including 1 and 166 of SEQ ID NOs 1, 2, 3 or 4. In some alternatives, the antibody is directed against a peptide having the amino acid sequence of SEQ ID NO: 1, 2, 3 or 4. In some alternatives, the antibody is Ab84716. In some alternatives, the antibody is a monoclonal antibody. In some alternatives, the monoclonal antibody is Ab85326, also named AF14A11. In some alternatives, the cancer is in the brain, endometrium, colon, blood, lung or epithelium/mouth. In some alternatives, the lung cancer is non-small cell lung carcinoma (NSCLC). In some alternatives, the cancer of the epithelium/mouth is a squamous cell carcinoma. In some alternatives, the cancer is a glioma. In some alternatives, the glioma is a glioblastoma. In some alternatives, the agent inhibits LDH-5 binding and/or incorporation and/or internalization and/or endocytosis to a lymphocyte, an NK Cell, an NKG2D-expressing cell and/or a monocyte. In some alternatives, the LDH-5 is extracellular. In some alternatives, the binding agent binds to LDH-5 without inhibiting or abolishing enzymatic activity. In some alternatives, the methods can further comprise administering targeted therapies. In some alternatives, the method can further comprise administering to a subject having cancer, photochemotherapy or photodynamic therapy. In some alternatives of the methods described herein, the antibody or binding fragment thereof is administered in combination with one or more other anti-cancer agents. In some alternatives, the one or more anti-cancer agent comprises antibody-drug conjugates, nanoparticles, electochemotherapy, alkylating agents, antimetabolites, anti-tumor antibiotics, topoisomerase inhibitors, mitotic inhibitors, corticosteroids, DNA intercalating agents, and checkpoint inhibitors. In some alternatives, the antimetabolites comprises 5-fluorouracil (5-FU), 6-mercaptopurine (6-MP), Capecitabine (Xeloda®), Cladribine, Clofarabine, Cytarabine (Ara-C®), Floxuridine, Fludarabine, Gemcitabine (Gemzar®), Hydroxyurea, Methotrexate, Pemetrexed (Alimta®), Pentostatin, or Thioguanine.

In some alternatives, a method of treating, inhibiting, or ameliorating cancer in a subject is provided, wherein the method comprises detecting LDH-5, or LDH-5 activity in a biological sample obtained from said subject, and administering to said subject a dose of an anti-LDH-5 antibody or binding fragment thereof raised against the amino terminus of said LDH-5 or a variant thereof such as, amino acid residues 1-241, 1-200, 1-175, 1-150, 1-125, 1-100, 1-75, 1-50, 1-25, 1-15, 1-10, or 1-5 or a range defined by any two numbers in between 1-241, including 1 and 241 of SEQ ID NOs 1, 2, 3 or 4. In some alternatives, the detection of LDH-5 is detection of LDH-5 itself, a subunit of LDH-5, or LDH-5 enzymatic activity. In some alternatives, the level or amount of LDH-5 protein or level or amount of LDH-5 activity in the biological sample is elevated relative to a control such as a biological sample obtained from a subject without cancer or with a benign tumor. In some alternatives, LDH-5 is detected in a biological sample selected from the group consisting of a serum sample, a tumor environment/microenvironment, a tissue sample from a tumor and a tissue sample from a region surrounding a tumor. In some alternatives, LDH-5 is detected in serum and binding of said antibody is to serum LDH-5. In some alternatives, the antibody or binding fragment thereof is administered in combination with one or more other anti-cancer agents. In some alternatives, said one or more anti-cancer agent is an antibody selected from the group consisting of an anti-MICB antibody, an anti-ULBP1 antibody and an anti-MICA antibody or binding fragments thereof can be used. In some alternatives, the agent is an anti-LDH-5 antibody, which inhibits LDH-5 binding to a receptor on an immune cell. In some alternatives, the one of more anti-cancer agent is an LDH-5 intracellular enzymatic inhibitor and/or an anti-NKG2d antibody, wherein the anti-NKG2d antibody blocks binding and/or ULBP1. In some alternatives, the methods can further comprise administering targeted therapies. In some alternatives, the method can further comprise administering to a subject having cancer, photochemotherapy or photodynamic therapy. In some alternatives of the methods described herein, the antibody or binding fragment thereof is administered in combination with one or more other anti-cancer agents. In some alternatives, the one or more anti-cancer agent comprises antibody-drug conjugates, nanoparticles, electochemotherapy, alkylating agents, antimetabolites, anti-tumor antibiotics, topoisomerase inhibitors, mitotic inhibitors, corticosteroids, DNA intercalating agents, and checkpoint inhibitors. In some alternatives, the antimetabolites comprises 5-fluorouracil (5-FU), 6-mercaptopurine (6-MP), Capecitabine (Xeloda®), Cladribine, Clofarabine, Cytarabine (Ara-C®), Floxuridine, Fludarabine, Gemcitabine (Gemzar®), Hydroxyurea, Methotrexate, Pemetrexed (Alimta®), Pentostatin, or Thioguanine.

In some alternatives, a method of treating, inhibiting, or ameliorating cancer in a subject is provided, wherein the method comprises administering to said subject a therapeutic dose of a nucleotide sequence encoding a peptide from the amino terminus of LDH-5. In some alternatives, the nucleotide sequence encodes a peptide having the amino acid sequence of SEQ ID NO: 1, SEQ ID NO: 2, SEQ ID NO: 3 or SEQ ID NO: 4.

In some alternatives, a method of treating, inhibiting, or ameliorating cancer in a subject is provided, wherein the method comprises detecting LDH-5, or LDH-5 activity in a biological sample obtained from said subject, administering to said subject a therapeutic dose of an anti-LDH-5 antibody or binding fragment thereof raised against the amino terminus of said LDH-5 or a variant thereof such as, amino acid residues 1-241, 1-200, 1-175, 1-150, 1-125, 1-100, 1-75, 1-50, 1-25, 1-15, 1-10, or 1-5 or a range defined by any two numbers in between 1-241, including 1 and 241 of SEQ ID NOs 1, 2, 3 or 4 and administering one or more other anti-cancer agents. In some alternatives, the detection of LDH-5 is detection of LDH-5 itself, a subunit of LDH-5, or LDH-5 enzymatic activity. In some alternatives, the level or amount of LDH-5 protein or level or amount of LDH-5 activity in the biological sample is elevated relative to a control such as a biological sample obtained from a subject without cancer or with a benign tumor. In some alternatives, LDH-5 is detected in a biological sample selected from the group consisting of a serum sample, a tumor environment/microenvironment, a tissue sample from a tumor and a tissue sample from a region surrounding a tumor. In some alternatives, LDH-5 is detected in serum and binding of said antibody is to serum LDH-5. In some alternatives, one or more anti-cancer agent is an antibody selected from the group consisting of an anti-MICB antibody, an anti-ULBP1 antibody and/or an anti-MICA antibody. In some alternatives, the methods can further comprise administering targeted therapies. In some alternatives, the method can further comprise administering to a subject having cancer, photochemotherapy or photodynamic therapy. In some alternatives of the methods described herein, the antibody or binding fragment thereof is administered in combination with one or more other anti-cancer agents. In some alternatives, the one or more anti-cancer agent comprises antibody-drug conjugates, nanoparticles, electochemotherapy, alkylating agents, antimetabolites, anti-tumor antibiotics, topoisomerase inhibitors, mitotic inhibitors, corticosteroids, DNA intercalating agents, and checkpoint inhibitors. In some alternatives, the antimetabolites comprises 5-fluorouracil (5-FU), 6-mercaptopurine (6-MP), Capecitabine (Xeloda®), Cladribine, Clofarabine, Cytarabine (Ara-C®), Floxuridine, Fludarabine, Gemcitabine (Gemzar®), Hydroxyurea, Methotrexate, Pemetrexed (Alimta®), Pentostatin, or Thioguanine.

In some alternatives, a method of stimulating an immune system in a subject with cancer is provided, wherein the method comprises selecting a subject to receive a binding agent specific for Lactate dehydrogenase 5 (LDH-5), or selecting a subject to receive an anti-cancer therapy, providing to said subject an amount of a binding agent, such as a fragment comprising a CDR domain or a Fab fragment, that is specific for LDH-5, preferably a binding agent that is specific for amino acid residues 1-241, 1-200, 1-175, 1-150, 1-125, 1-100, 1-75, 1-50, 1-25, 1-15, 1-10, or 1-5 or a range defined by any two numbers in between 1-241, including 1 and 241 of SEQ ID NOs 1, 2, 3 or 4, preferably said binding agent binds to LDH-5 and optionally, observing, analyzing, monitoring, or measuring an inhibition, amelioration, or treatment of cancer in said subject. In some alternatives, the binding agent is an anti-LDH-5 antibody or binding fragment thereof, such as a fragment comprising a CDR domain or a Fab fragment, preferably a monoclonal antibody or binding fragment thereof, such as a fragment comprising a CDR domain or a Fab fragment. In some alternatives, the anti-LDH-5 antibody or binding fragment thereof is directed to the amino terminus of LDH-5, such as amino acid residues 1-166, 1-150, 1-125, 1-100, 1-75, 1-50, 1-25, 1-15, 1-10, or 1-5 or a range defined by any two numbers in between 1-166, including 1 and 166 of SEQ ID NOs 1, 2, 3 or 4. In some alternatives, the antibody is directed against a peptide having the amino acid sequence of SEQ ID NO: 1, 2, 3 or 4. In some alternatives, the antibody is Ab84716. In some alternatives, antibody is a monoclonal antibody. In some alternatives, the monoclonal antibody is Ab85326, also named AF14A11. In some alternatives, the cancer is in the brain, endometrium, colon, blood, lung or epithelium/mouth. In some alternatives, the lung cancer is non-small cell lung carcinoma (NSCLC). In some alternatives, the cancer of the epithelium/mouth is a squamous cell carcinoma. In some alternatives, the cancer is a glioma. In some alternatives, the agent inhibits LDH-5 binding and/or incorporation and/or internalization and/or endocytosis to a lymphocyte, an NK Cell, an NKG2D-expressing cell and/or a monocyte. In some alternatives, the LDH-5 is extracellular. In some alternatives, the antibody or binding fragment thereof is administered in combination with one or more other anti-cancer agents. In some alternatives, the one of more anti-cancer agent is an LDH-5 intracellular enzymatic inhibitor and/or an anti-NKG2d antibody, wherein the anti-NKG2d antibody blocks binding and/or ULBP1. In some alternatives, the methods can further comprise administering targeted therapies. In some alternatives, the method can further comprise administering to a subject having cancer, photochemotherapy or photodynamic therapy. In some alternatives of the methods described herein, the antibody or binding fragment thereof is administered in combination with one or more other anti-cancer agents. In some alternatives, the one or more anti-cancer agent comprises antibody-drug conjugates, nanoparticles, electochemotherapy, alkylating agents, antimetabolites, anti-tumor antibiotics, topoisomerase inhibitors, mitotic inhibitors, corticosteroids, DNA intercalating agents, and checkpoint inhibitors. In some alternatives, the antimetabolites comprises 5-fluorouracil (5-FU), 6-mercaptopurine (6-MP), Capecitabine (Xeloda®), Cladribine, Clofarabine, Cytarabine (Ara-C®), Floxuridine, Fludarabine, Gemcitabine (Gemzar®), Hydroxyurea, Methotrexate, Pemetrexed (Alimta®), Pentostatin, or Thioguanine.

### Tumor Immune Evasion by Lactate Dehydrogenase Induction of NKG2D Ligands on Myeloid Cells in Cancer Patients.

Tumor-infiltrating myeloid cells (TIMCs) are key regulators of the tumor microenvironment, governing local immune responses. Here it was shown that NKG2D ligands MICB and ULBP-1 are expressed on TIMCs and circulating blood monocytes in patients with glioblastoma multiform (GBM), impairing NKG2D receptor-dependent Natural Killer (NK) cell-mediated immune responses and that tumor derived lactate dehydrogenase (LDH) isoform 5 induces transcription of ligands in monocytes from healthy individuals. LDH in the sera of GBM patients correlates with expression of NKG2D ligands on circulating monocytes, and NK cells isolated from tumors have immune dysfunction. NKG2D ligands are also present on circulating monocytes isolated from patients with breast, prostate, and HCV-induced hepatocellular carcinomas, thus revealing a general immune evasion strategy whereby tumors produce soluble factors that induce NKG2D ligands on myeloid cells, subverting anti-tumor responses.

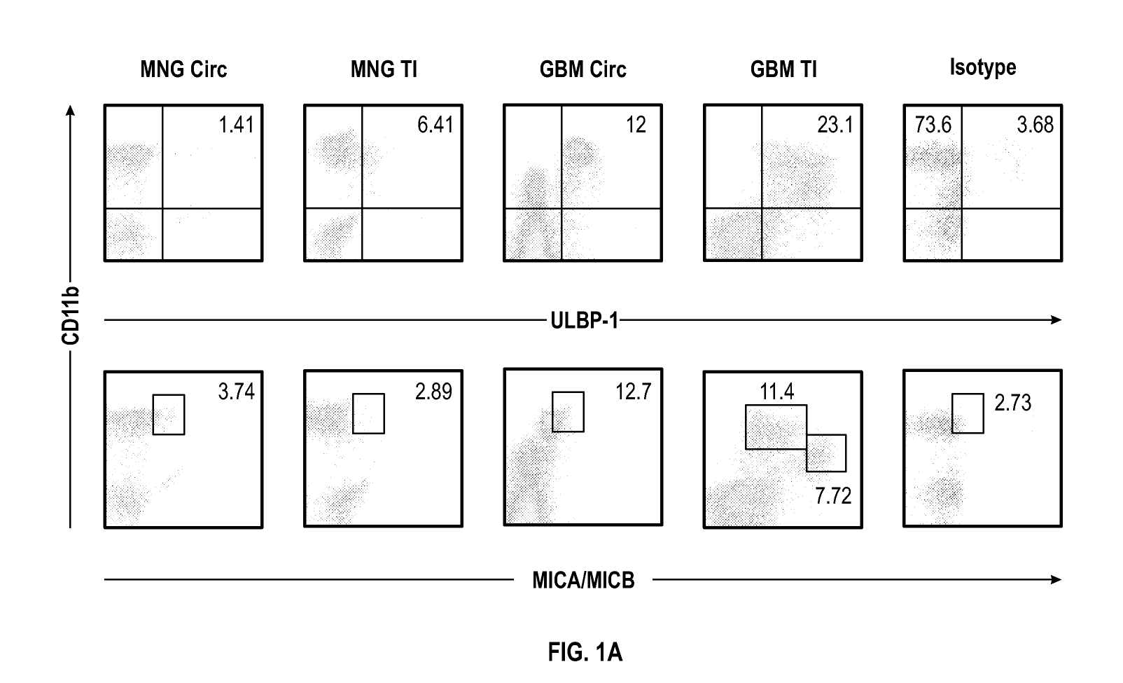

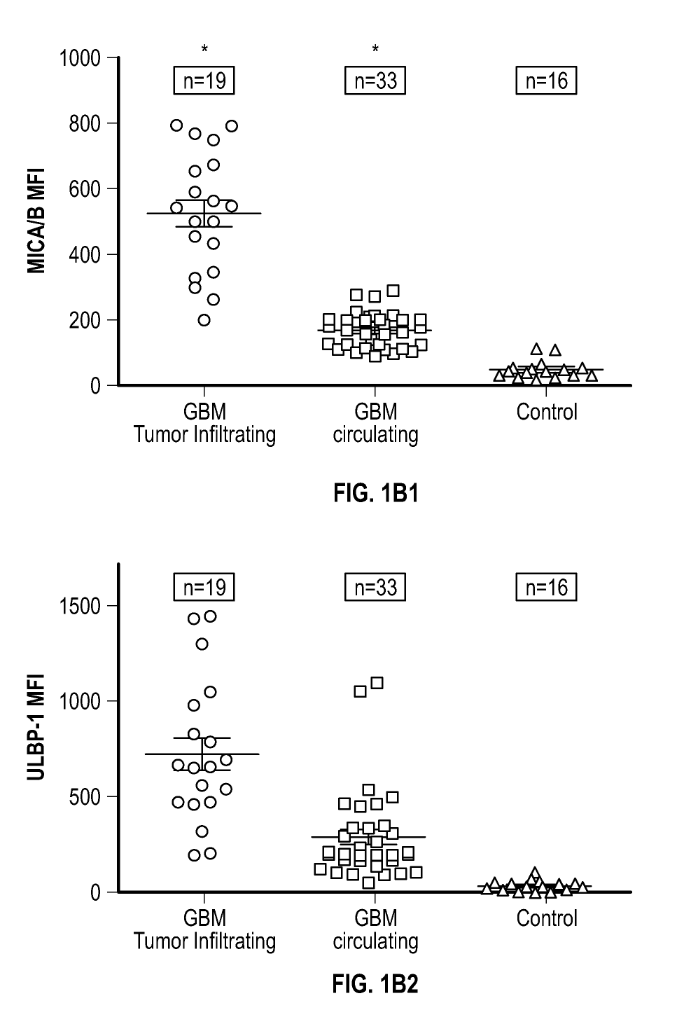

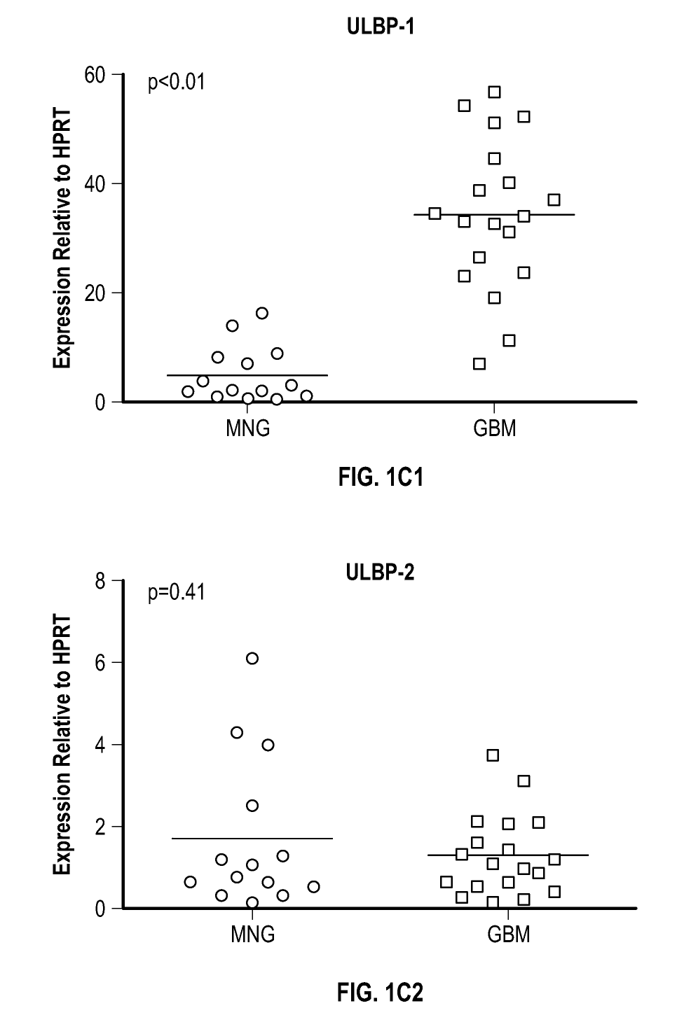

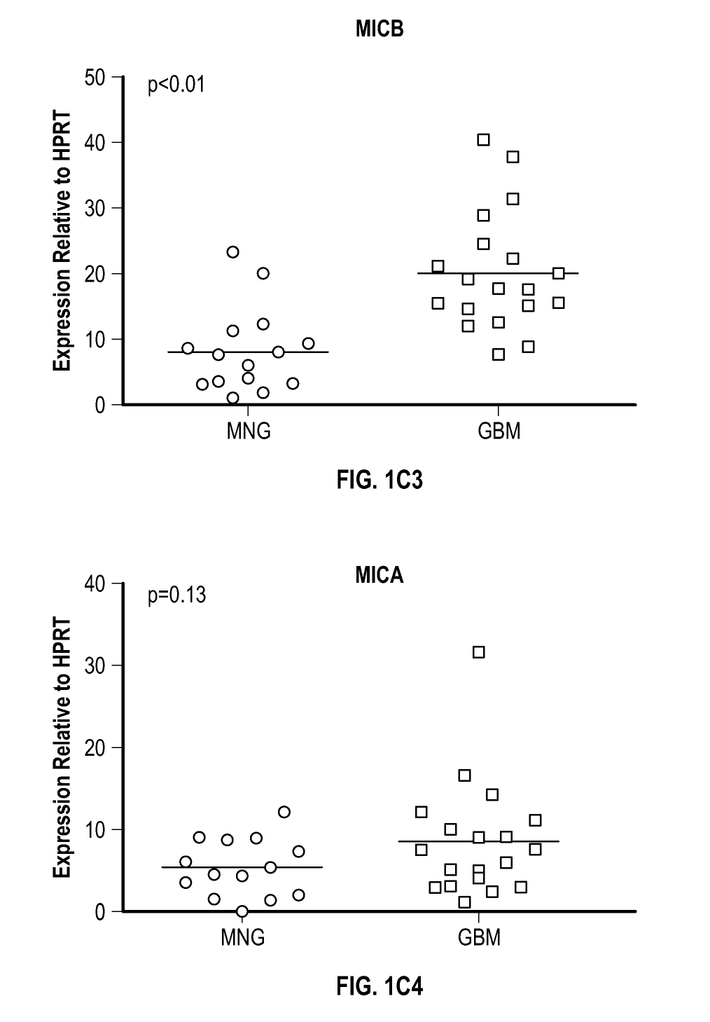

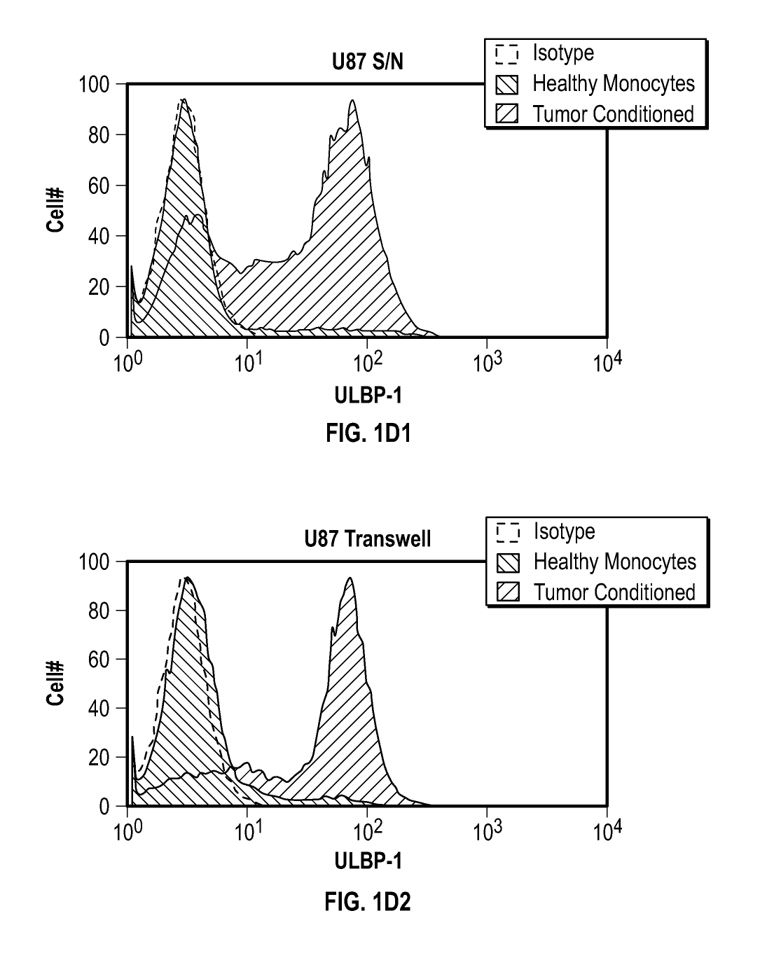

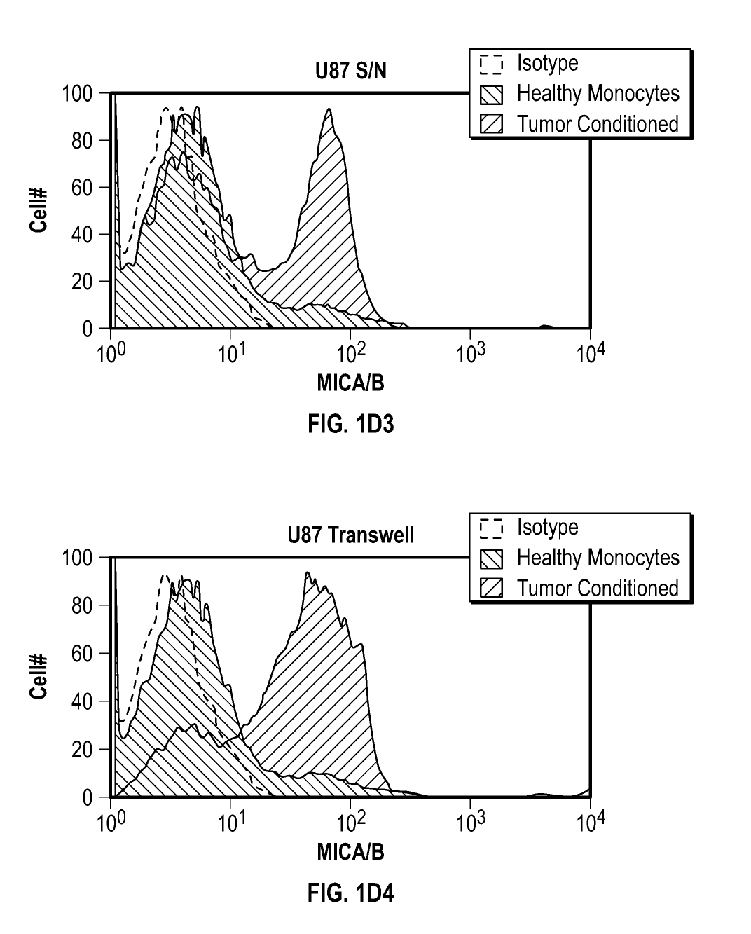

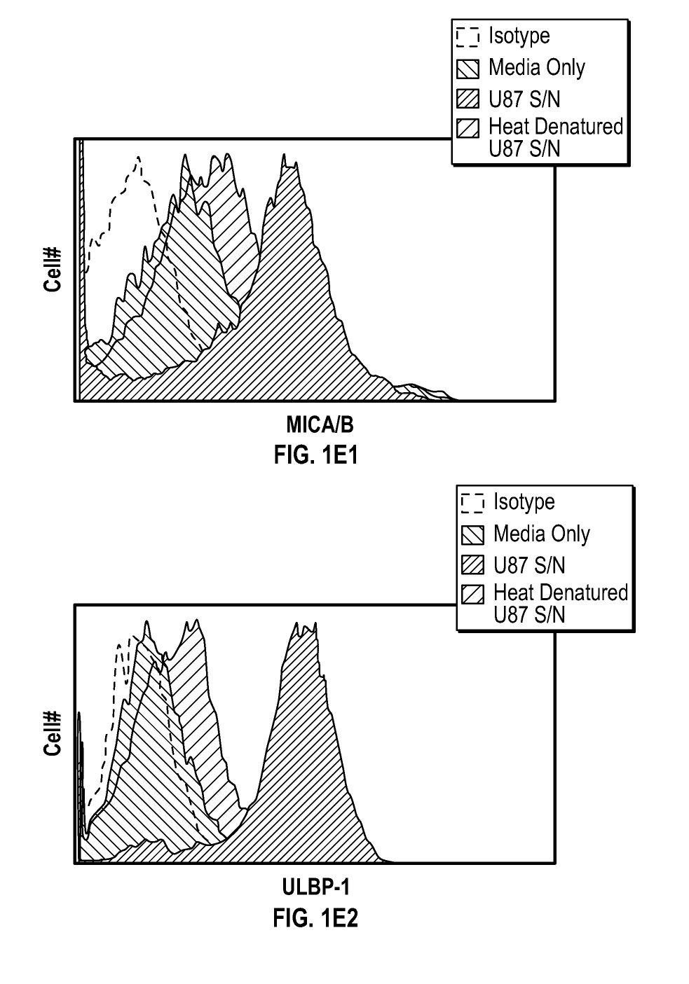

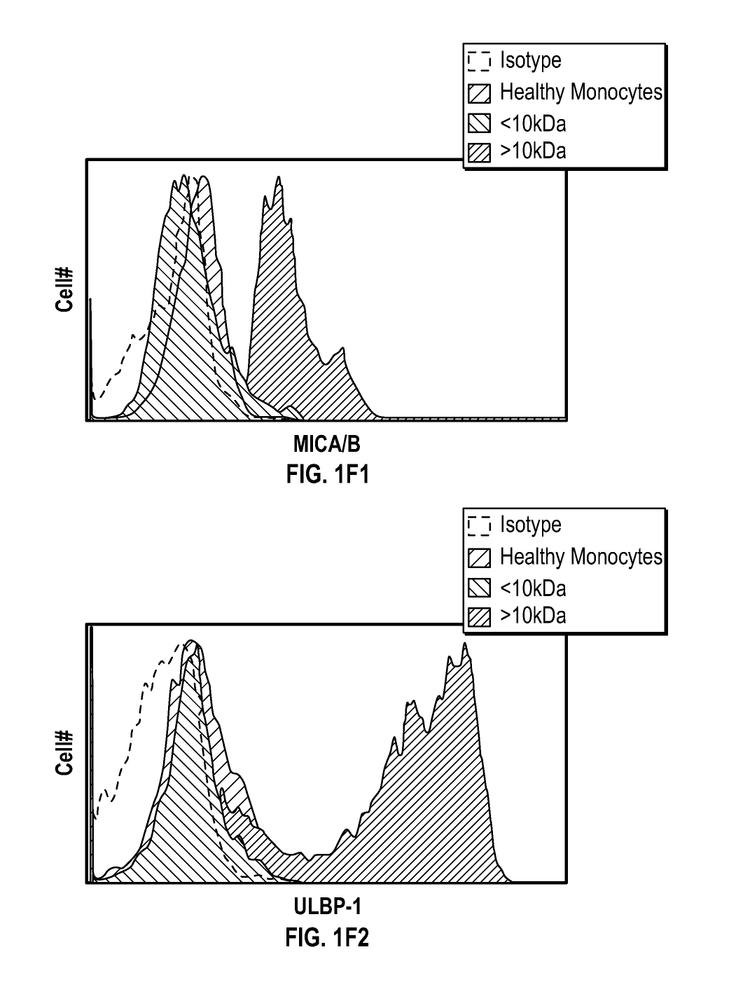

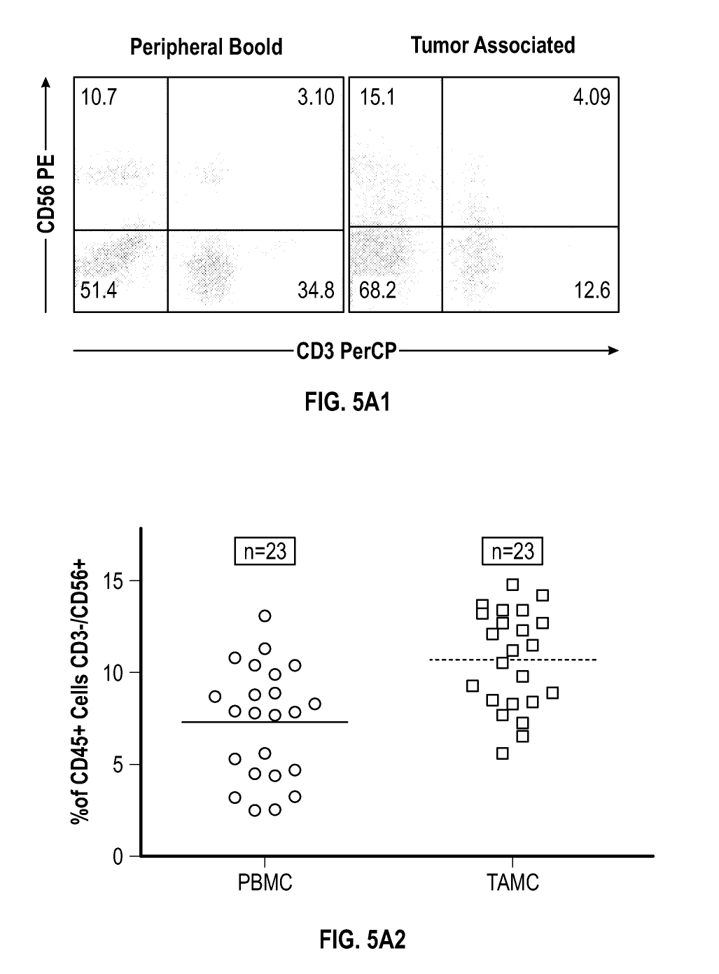

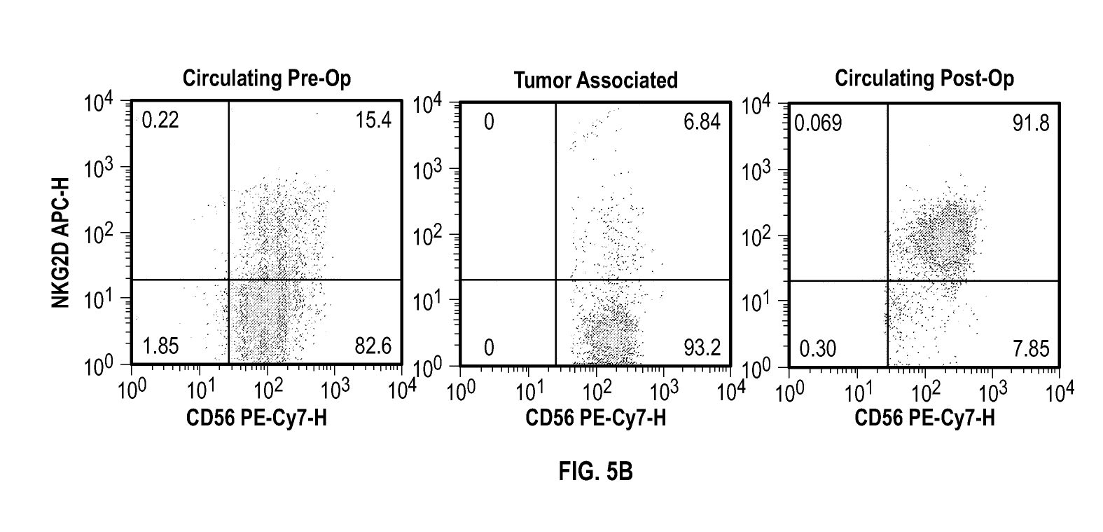

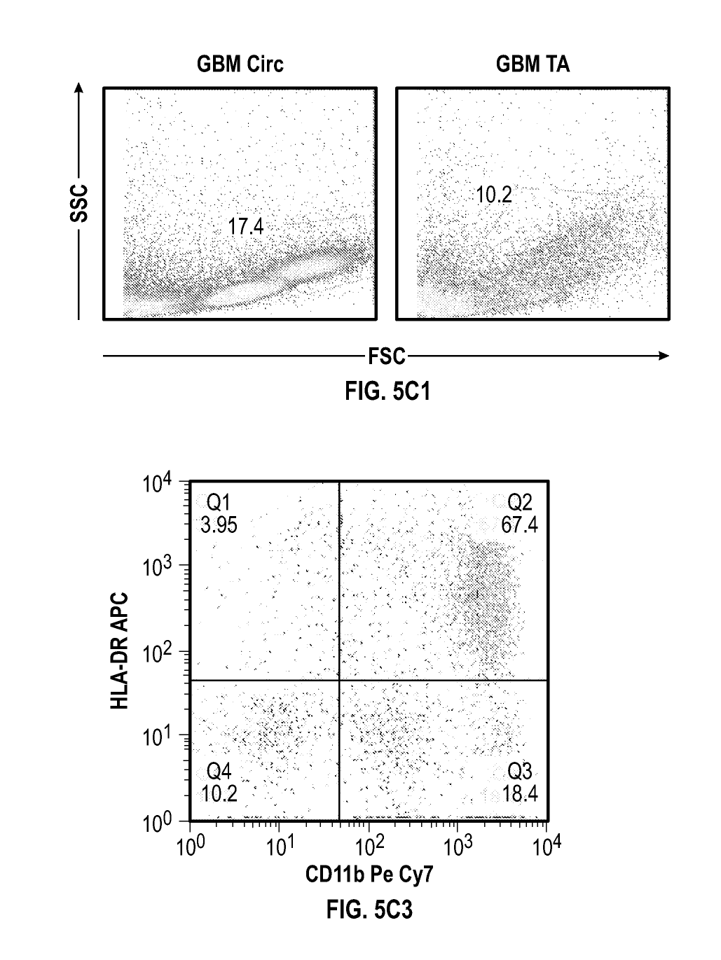

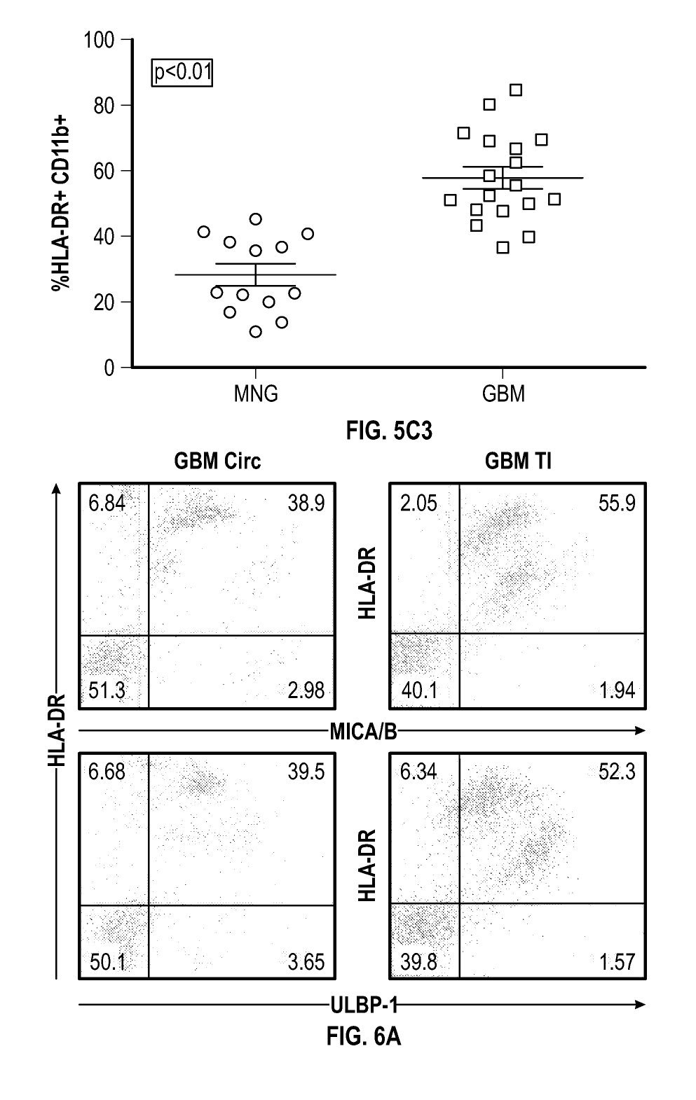

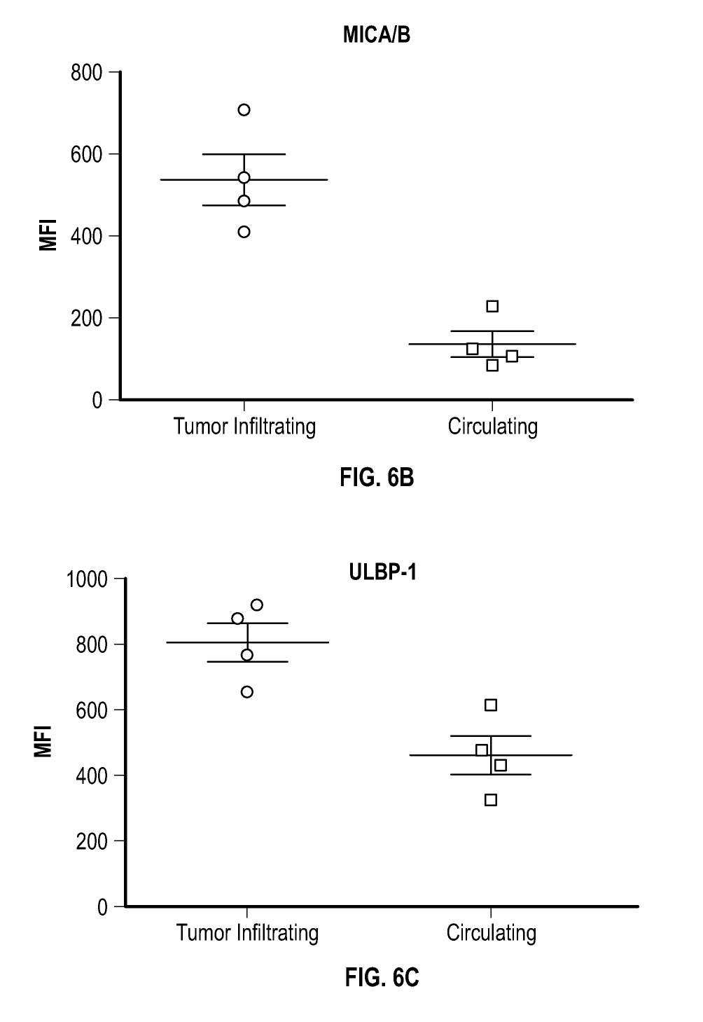

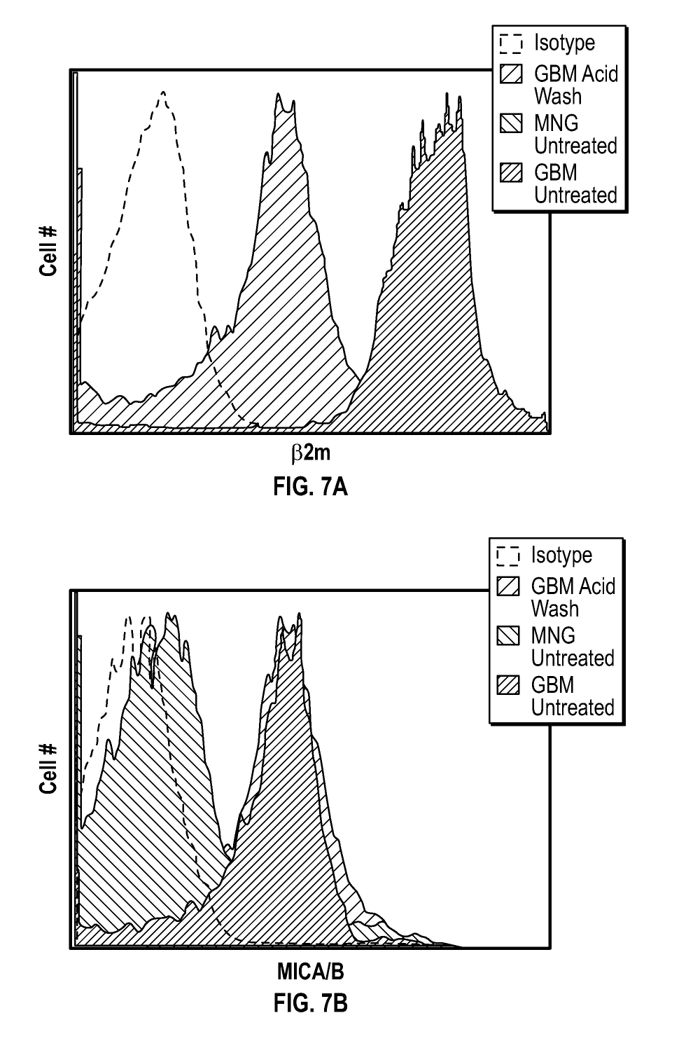

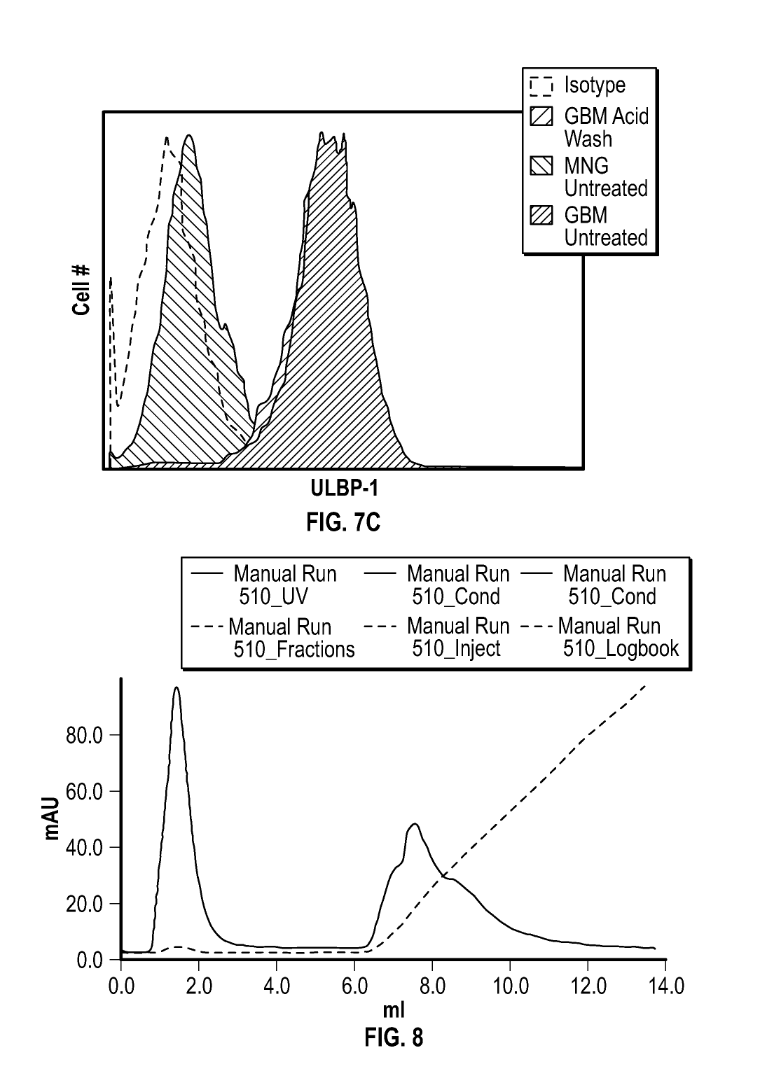

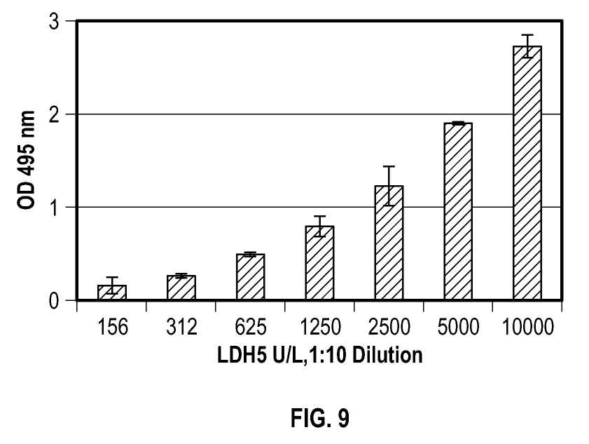

It is also evident that tumor cells in GBM patients express NKG2D ligands, and that circulating and tumor-infiltrating NK cells have decreased NKG2D (Crane, C. A, et al. TGF-beta downregulates the activating receptor NKG2D on NK cells and CD8+ T cells in glioma patients. Neuro-oncology 12, 7-13 (2010)) (FIG. 5B), despite detection of NK cells in the tumor site at a frequency similar to NK cells in peripheral blood (FIG. 5A). Myeloid cells are the most prevalent leukocytes infiltrating tumors in GBM patients (FIG. 5C); 57.6% (n=18) of CD45-positive cells among GBM tumor-infiltrating leukocytes were CD11b high myeloid cells as compared to 28.28% (n=13) in patients with benign intracranial meningioma (MNG) (FIG. 5C). In GBM patients, TIMCs (CD11b high macrophages and CD11blow CNS-resident microglia) and circulating monocytes expressed at least two of the NKG2D ligands, MICB and ULBP-1 (n=33), which were not detected in MNG patients (n=16) (FIG. 1A, 1B). Patients with newly diagnosed GBM, analyzed before receiving any therapy, expressed MICB and ULBP-1 on circulating monocytes, indicating that expression was independent of treatment (FIG. 6, n=4). Transcriptional analysis confirmed that NKG2D ligands were not passively acquired (Groh, V., Wu, J., Vee, C. & Spies, T. Tumour-derived soluble MIC ligands impair expression of NKG2D and T-cell activation. Nature 419, 734-738 (2002)), revealing endogenous transcription of MICB and ULBP-1 in GBM patient monocytes (FIG. 1C, n=19). Of eight NKG2D ligands, only ULBP-1 and MICB were expressed by monocytes and TIMCs in all GBM patients (data not shown, n=16). Acid washing of monocytes removed β2-microglobulin, a surface protein lacking a transmembrane domain, but not MICB or ULBP-1, indicating that NKG2D ligands are endogenously expressed (FIG. 7).

Given their expression on circulating monocytes, it was hypothesized that the NKG2D ligands were induced by a tumor-derived soluble factor. Using cell-free supernatant or Transwell assays, it was determined that soluble products of the U87 glioma cell line induced expression of MICB and ULBP-1 on monocytes from healthy blood donors (MHD) (FIG. 1D). Heat denaturation (FIG. 1E) and size-exclusion dialysis (FIG. 1F) revealed that a heat labile factor >10 kDa induced MICB and ULBP-1 expression.

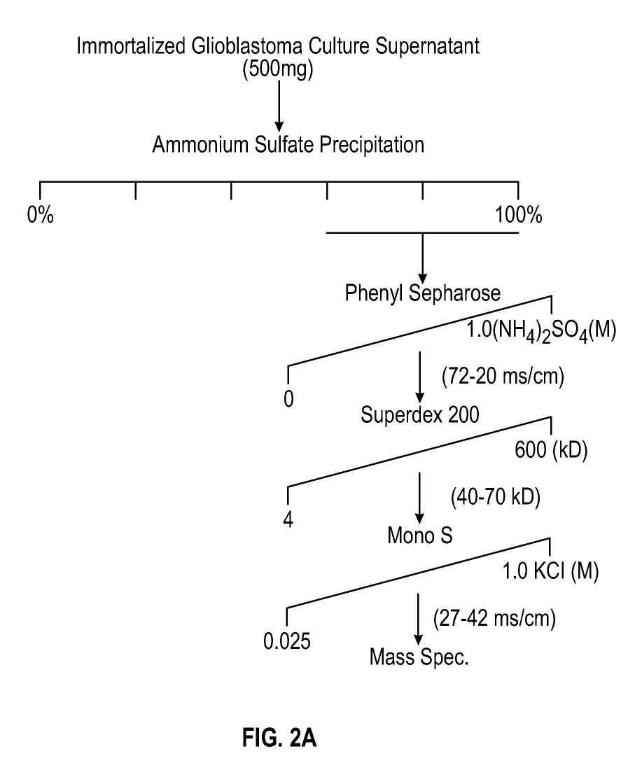

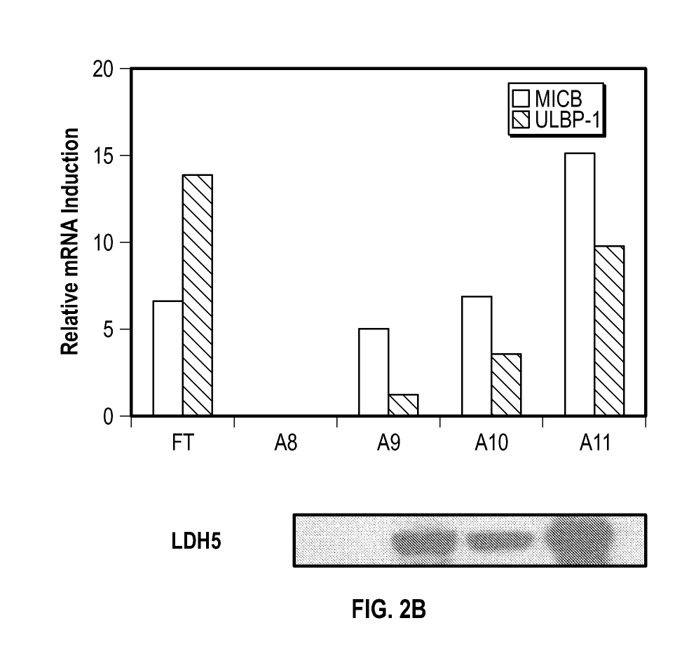

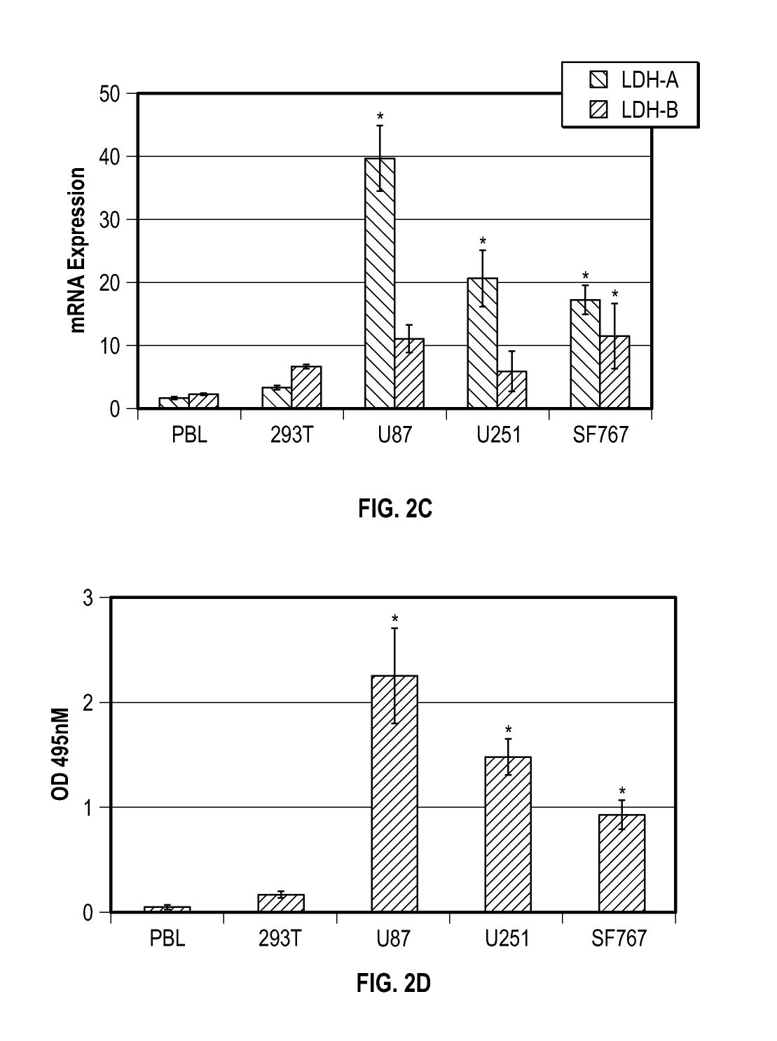

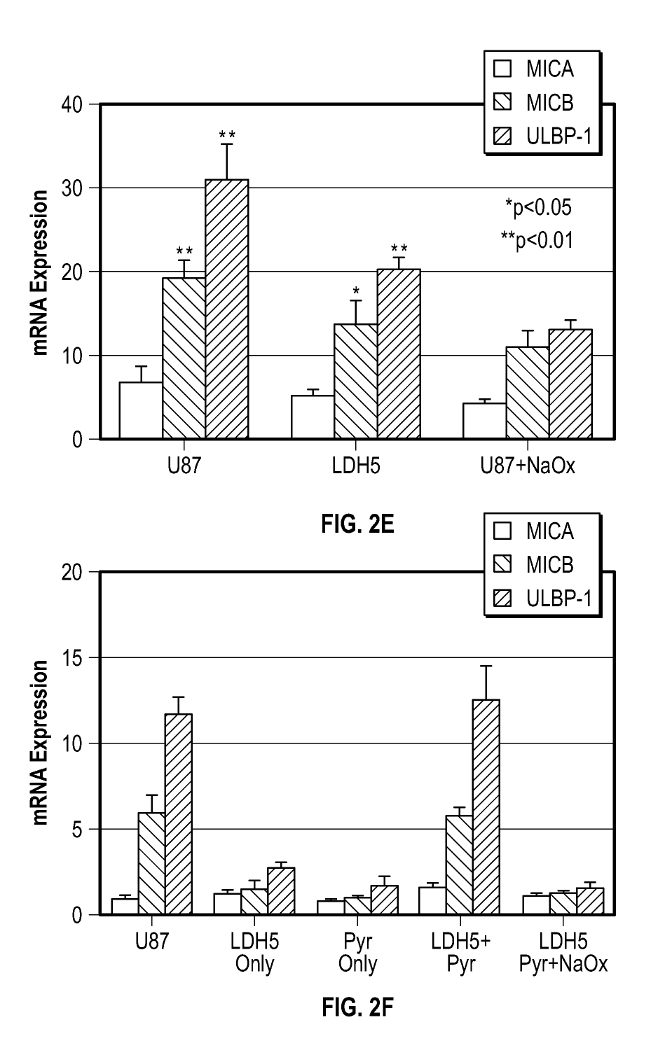

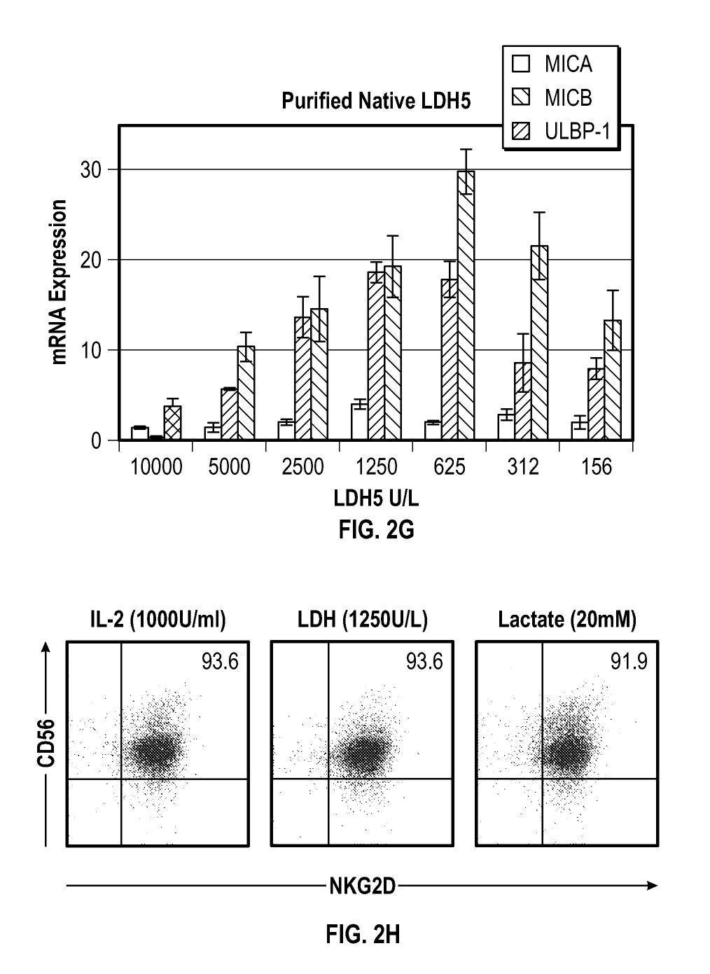

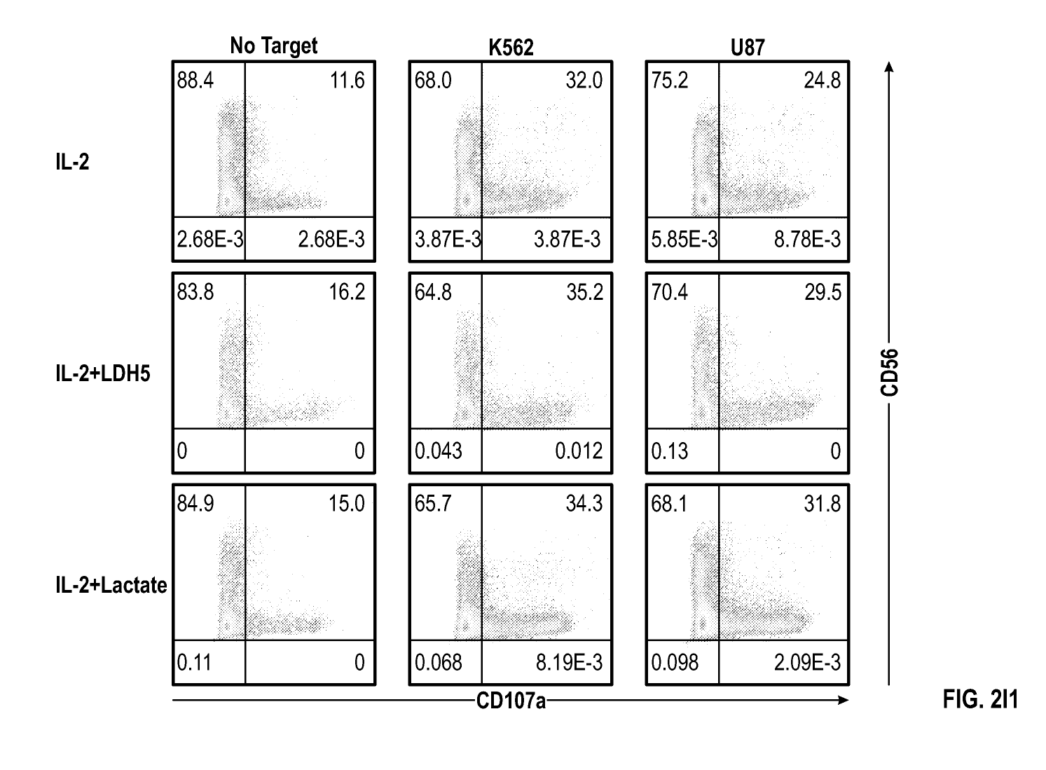

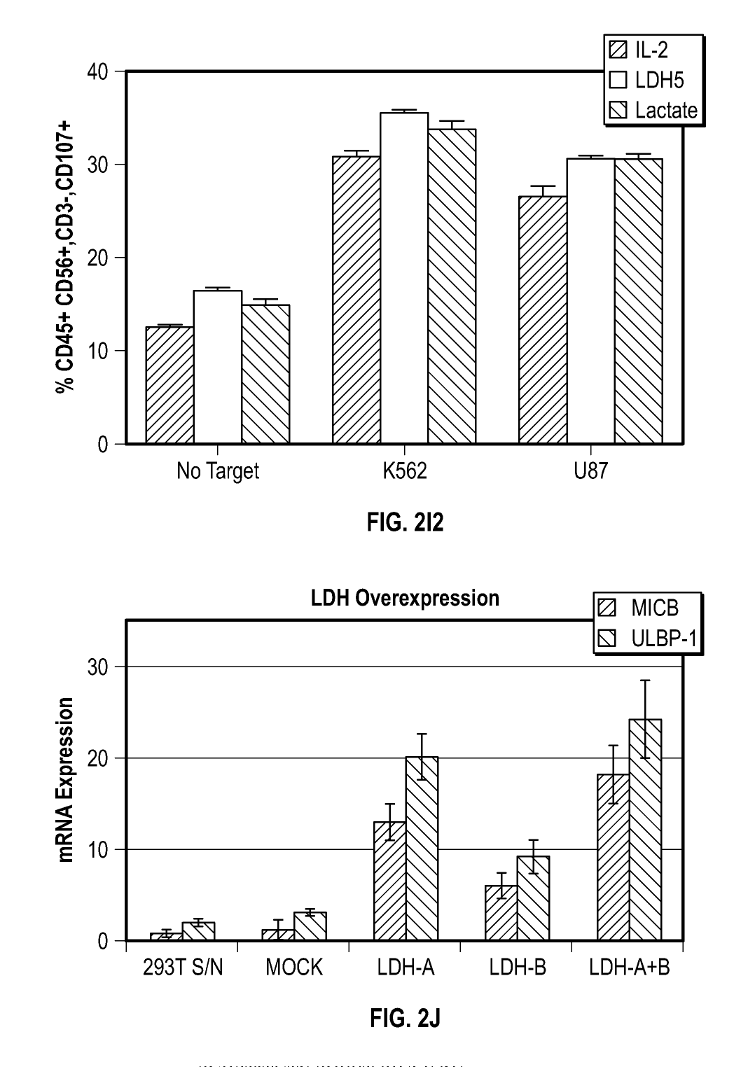

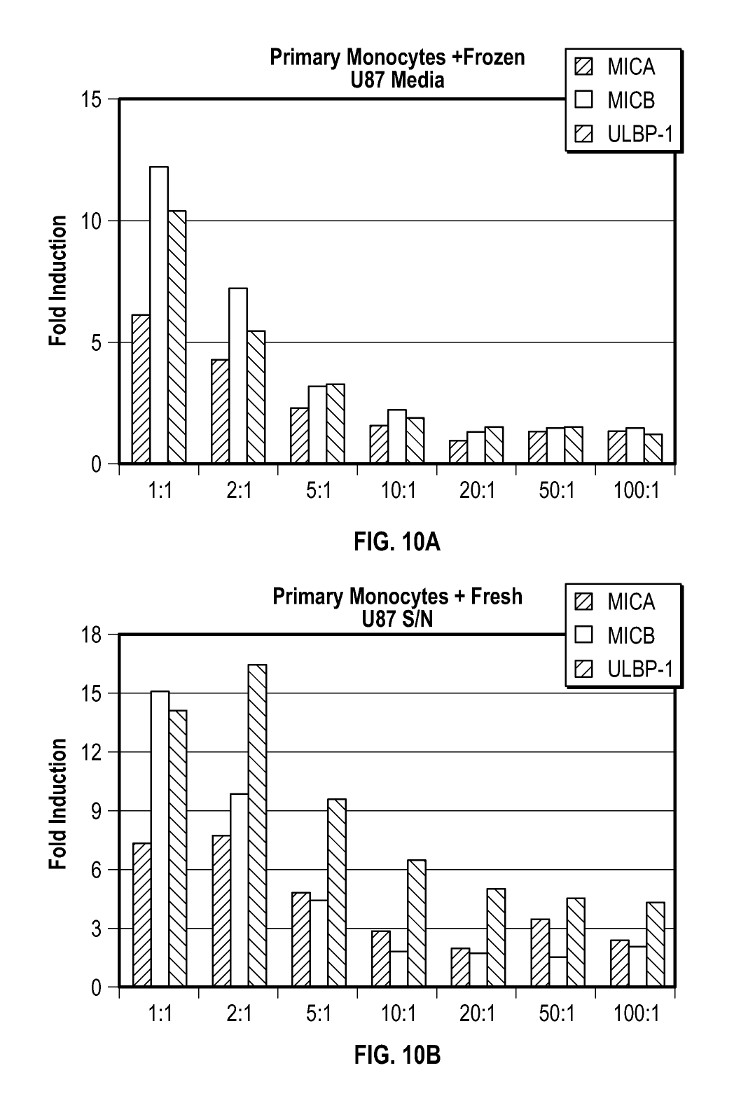

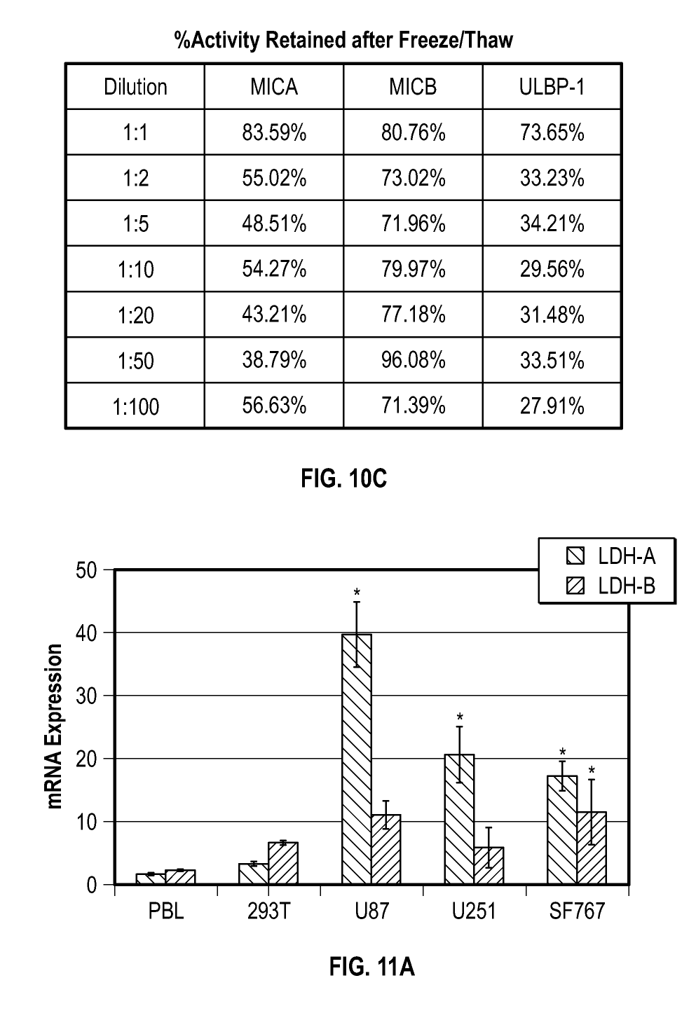

Unbiased protein purification based on in vitro induction of MICB and ULBP-1 transcription in MHD and mass spectrometry identified an isoform of Lactate Dehydrogenase (LDH-5) as the principal component that induced NKG2D ligand expression in MHD (FIG. 2 panel A, 2 panel B, and FIG. 8). Lactate dehydrogenase is a tetrameric metabolic enzyme that promotes ATP production in resource-deprived environments. There are five isoforms of LDH, consisting of different ratios of alpha and beta subunits with varied tissue distribution. Consistent with prior reports of correlations between elevated LDH-5 in sera of cancer patient and poor prognoses (Giatromanolaki, A, et al. Lactate dehydrogenase 5 (LDH-5) expression in endometrial cancer relates to the activated VEGF/VEGFR2 (KDR) pathway and prognosis. Gynecologic oncology 103, 912-918 (2006); Kolev, Y., Uetake, H., Takagi, Y. & Sugihara, K. Lactate dehydrogenase-5 (LDH-5) expression in human gastric cancer: association with hypoxia-inducible factor (HIF-1alpha) pathway, angiogenic factors production and poor prognosis. Annals of surgical oncology 15, 2336-2344 (2008); Koukourakis, M. L, Giatromanolaki, A, Sivridis, E., Gatter, K. C. & Harris, A L. Lactate dehydrogenase 5 expression in operable colorectal cancer: strong association with survival and activated vascular endothelial growth factor pathway—a report of the Tumour Angiogenesis Research Group. Journal of clinical oncology: official journal of the American Society of Clinical Oncology 24, 4301-4308 (2006); and Koukourakis, M. L, Kontomanolis, E., Giatromanolaki, A, Sivridis, E. & Liberis, V. Serum and tissue LDH levels in patients with breast/gynaecological cancer and benign diseases. Gynecologic and obstetric investigation 67, 162-168 (2009)), these findings indicate that LDH-5 can promote immune escape of tumor cells. Glioma cell lines U87, U251, and SF767 transcribe significantly more LDHa and LDHP than MHD and 293T cells (FIG. 2 panel C), and supernatants of these cell lines contained active LDH (FIG. 2 panel D and FIG. 10). Sodium oxamate, a substrate analog that prevents LDH activity, significantly reduced the ability of U87 supernatant to induce NKG2D ligands on MHD (FIG. 2 panel E), and NKG2D ligand induction required LDH-5 enzymatic activity and its substrate, pyruvate (FIG. 2 panel F). Conversely, it was examined whether LDH-5 is sufficient to induce NKG2D ligands on MHD. Healthy individuals have LDH-5 serum concentrations of <150 U/L (Copur, S., et al. Lactate dehydrogenase and its isoenzymes in serum from patients with multiple myeloma. Clinical chemistry 35, 1968-1970 (1989)). Concentrations of purified native LDH-5 as low as 156 U/L induced transcription of MICB and ULBP-1 in MHD (FIG. 2 panel G), but neither lactate nor LDH-5 alone directly impacted NK cell NKG2D expression or degranulation (FIG. 2 panel H, 2 panel I). Supernatants from 293T cells transfected with plasmids encoding either the LDHa or LDHβ subunits induced transcription of MICB and ULBP-1 in MHD (FIG. 2 panel J).

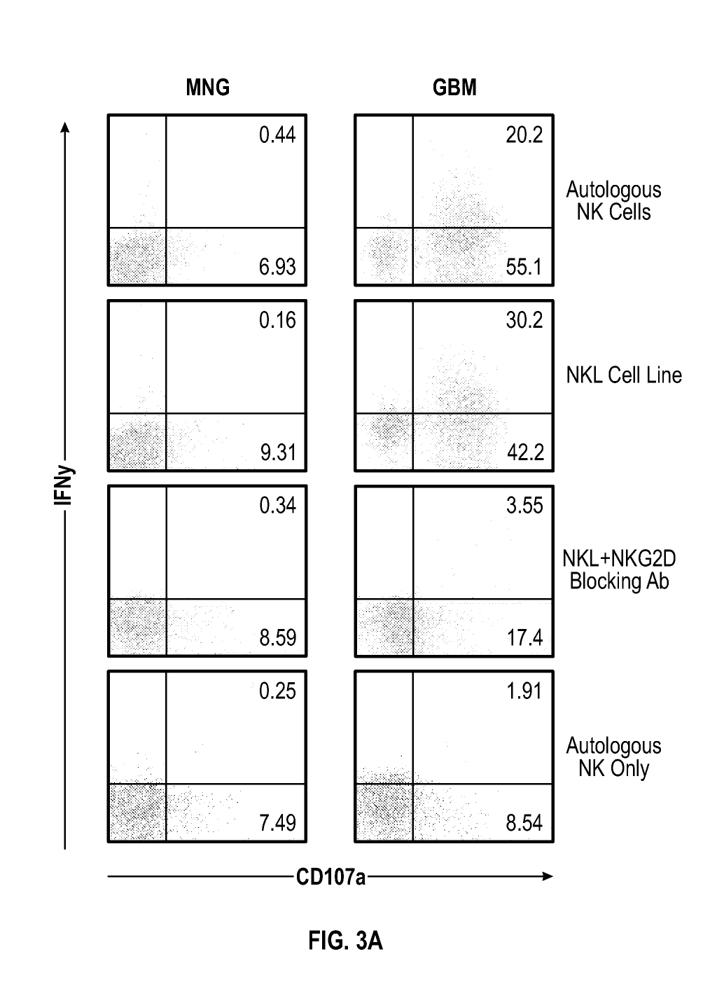

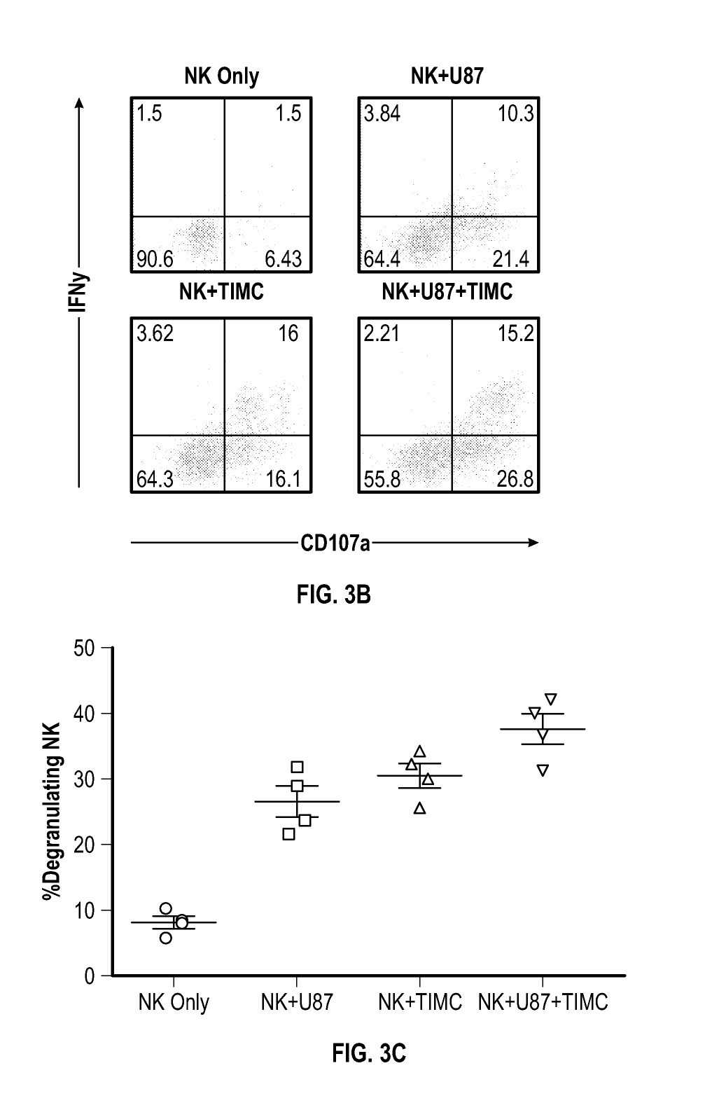

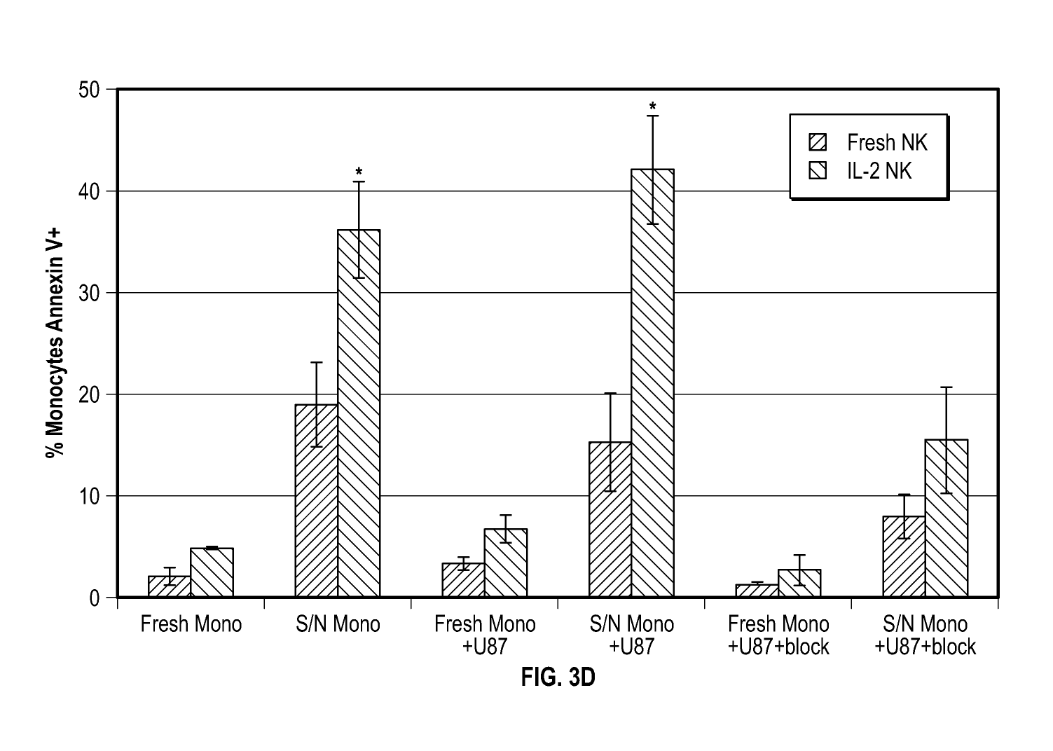

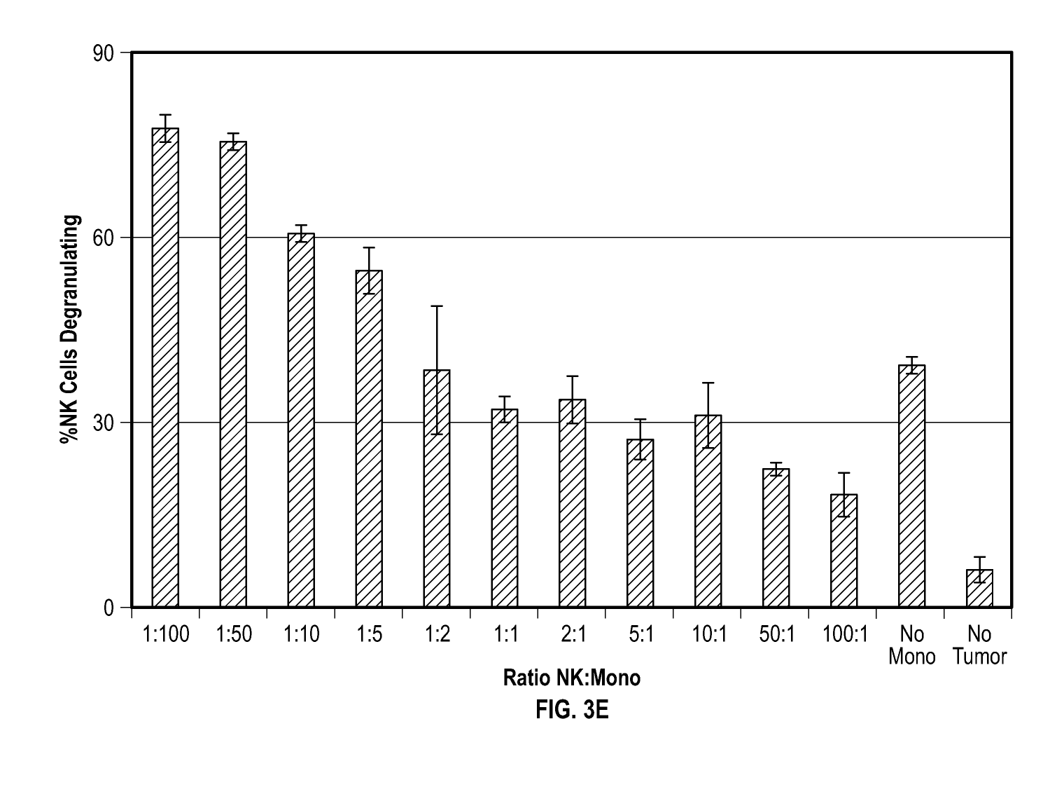

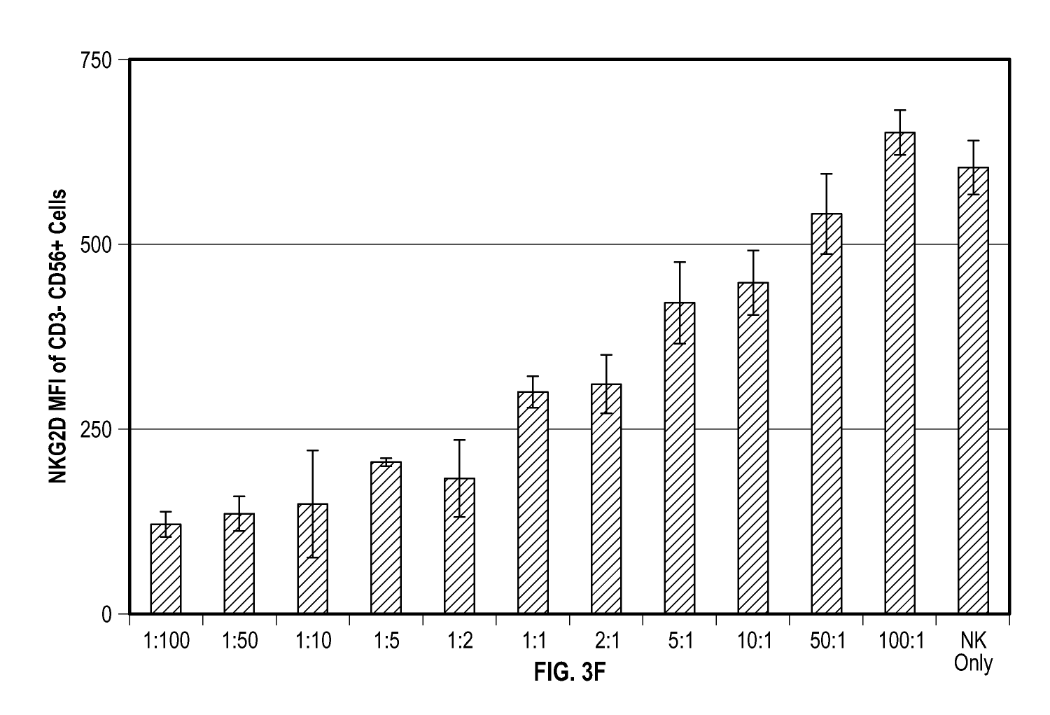

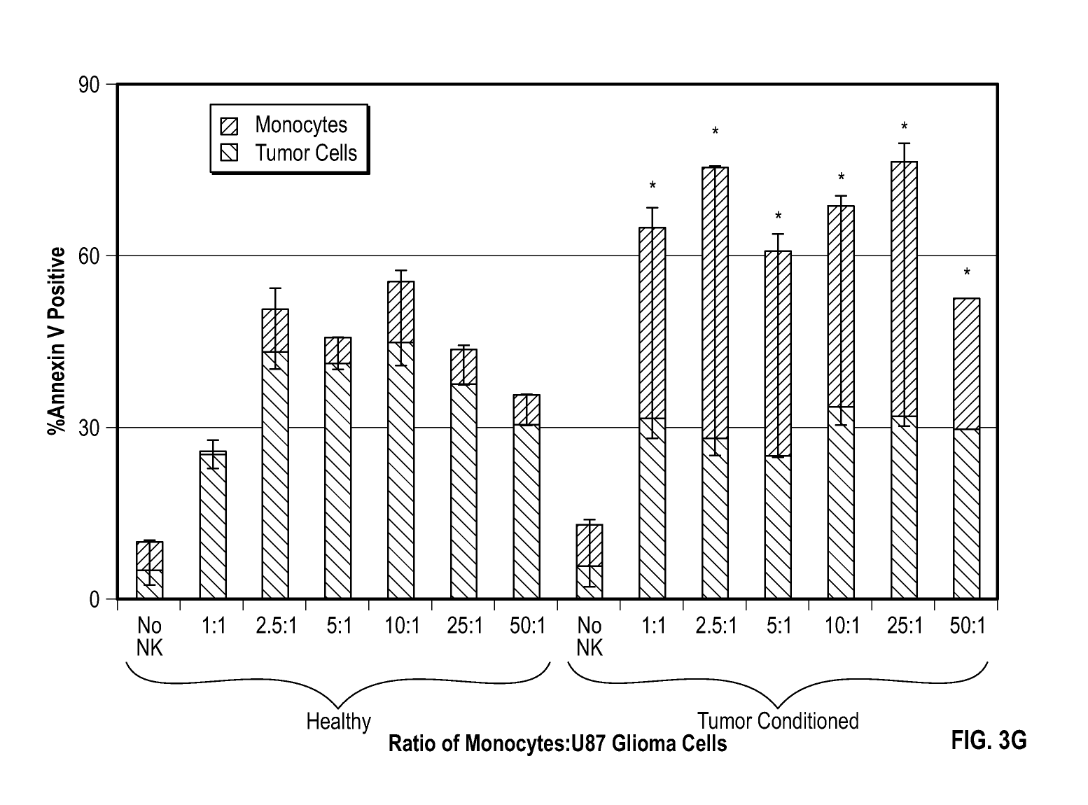

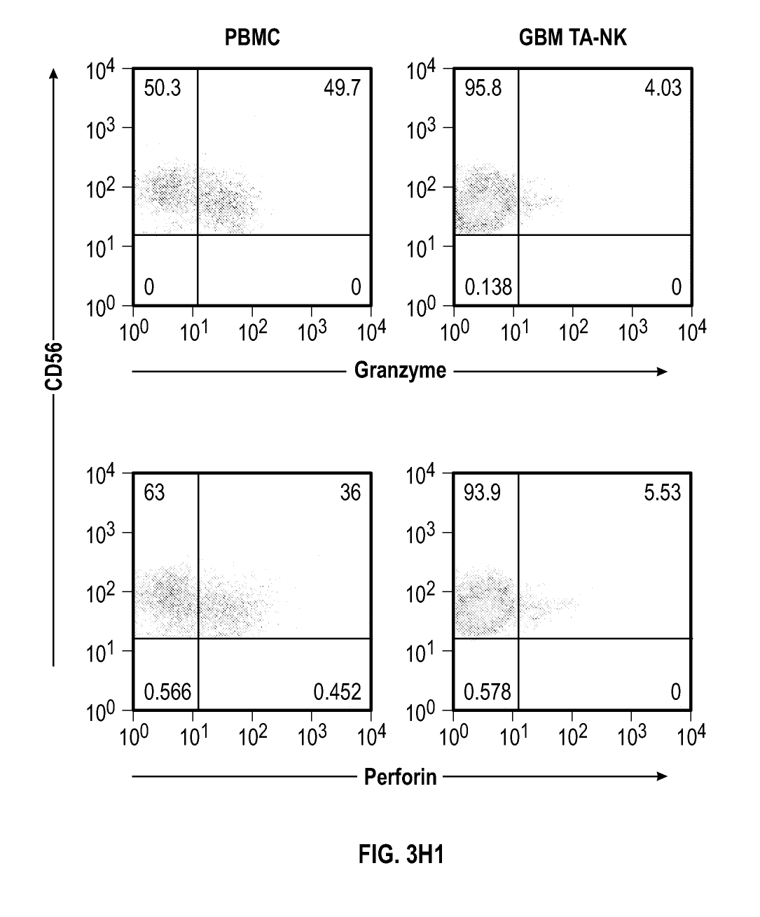

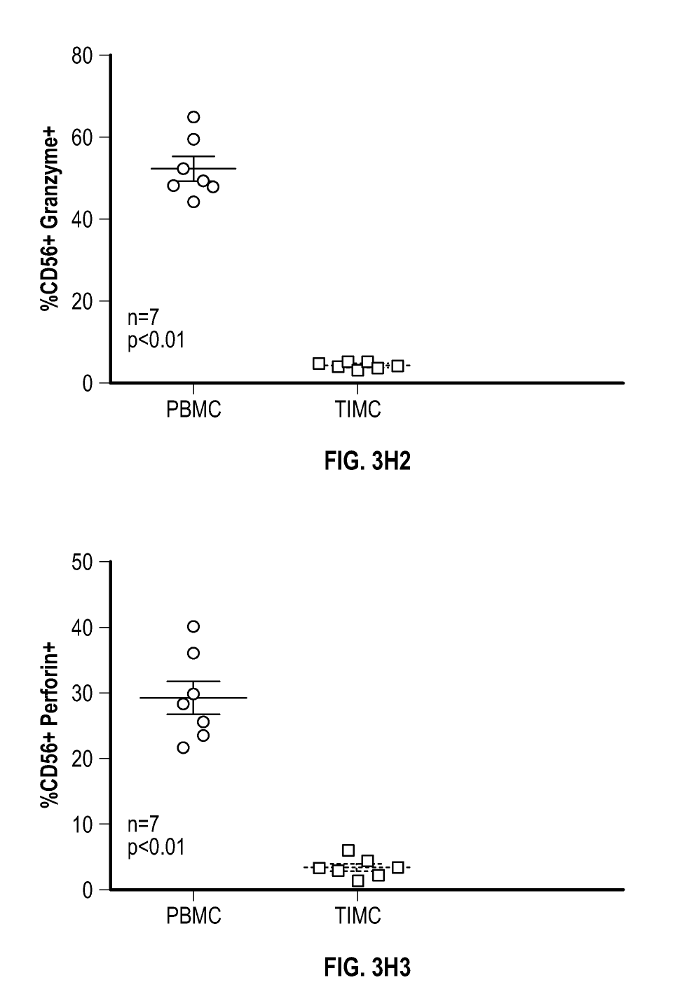

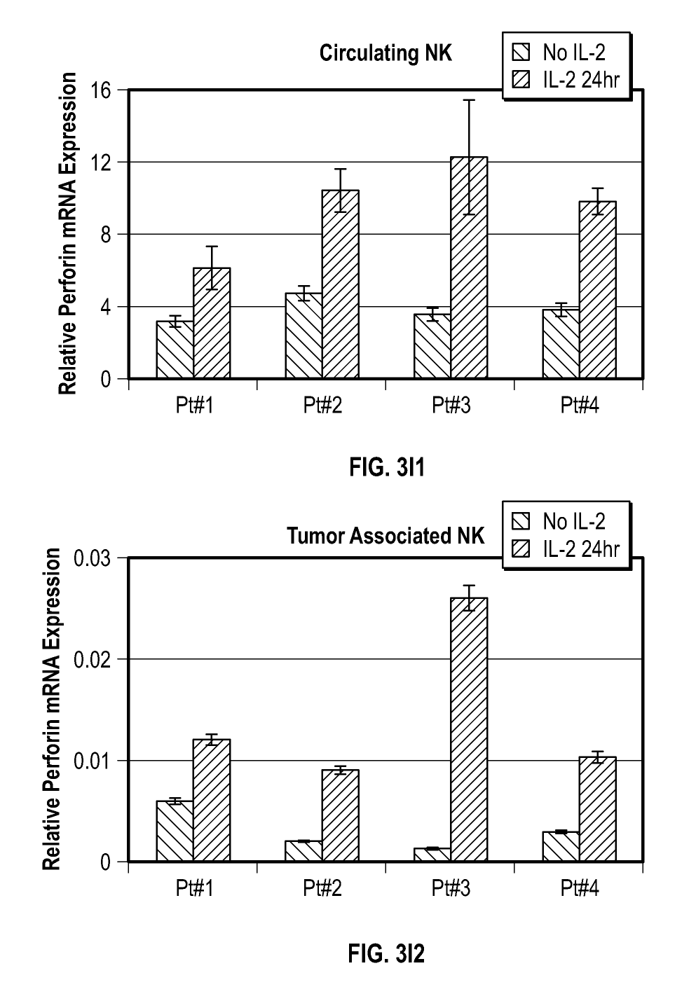

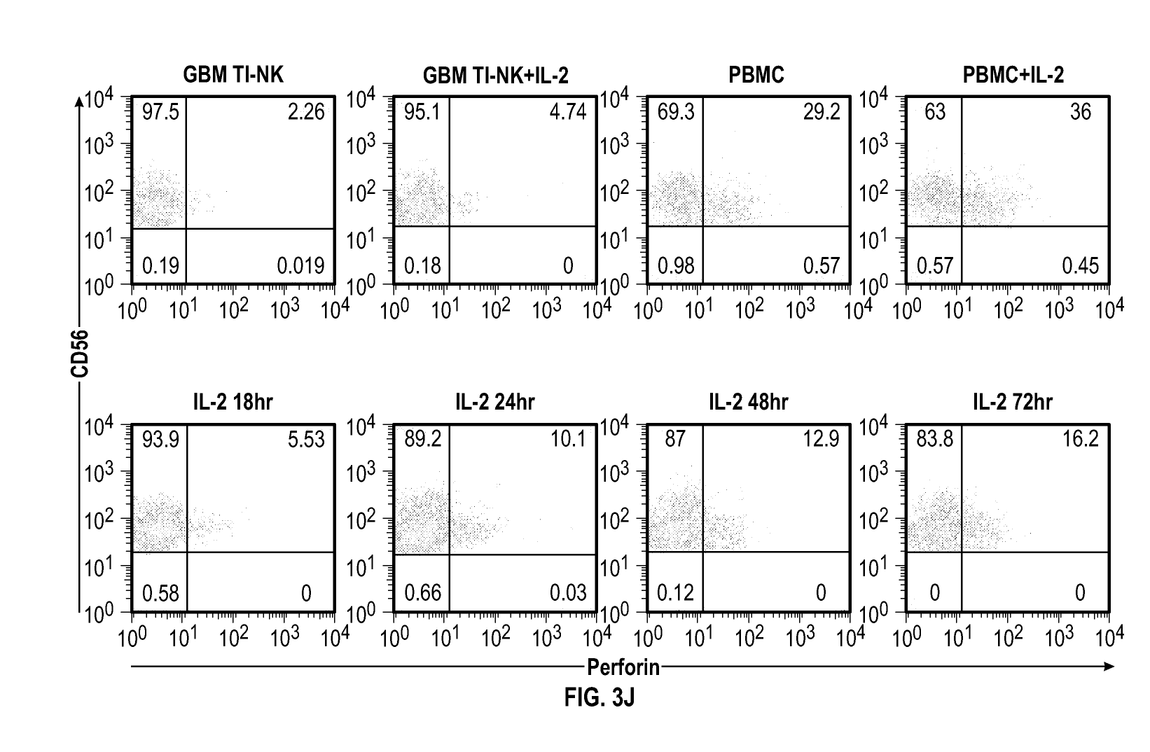

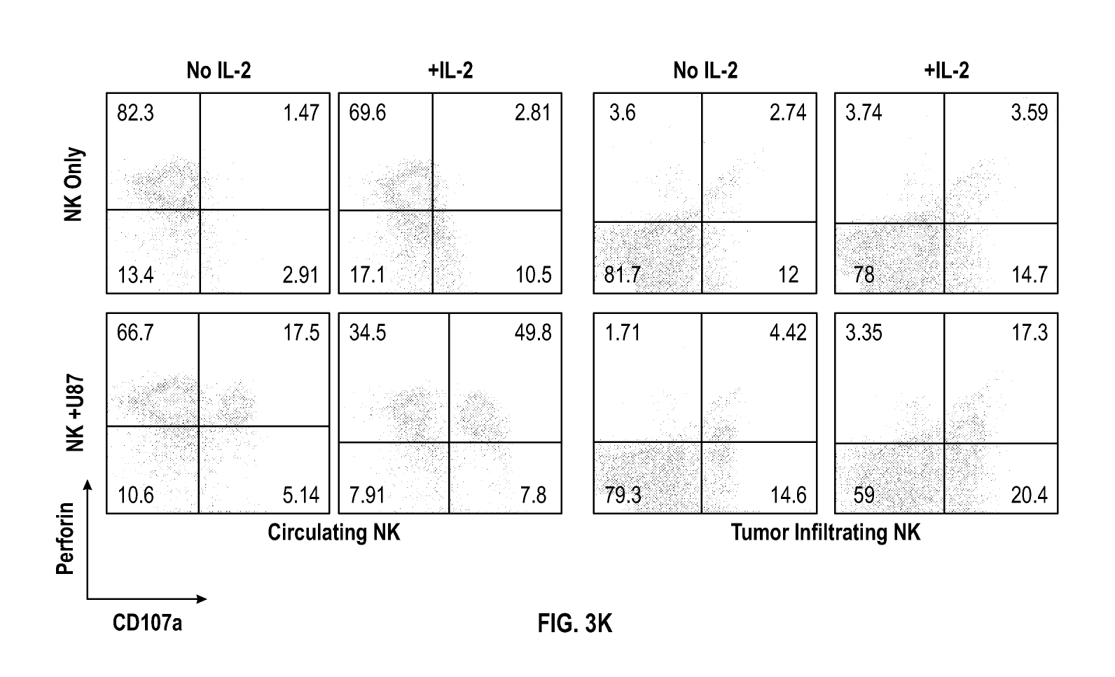

The functional consequences of NKG2D ligand expression by monocytes were then examined. When monocytes isolated from GBM patients were co-cultured with autologous IL-2-activated NK cells, they induced degranulation and interferon-gamma production of the NK cells (FIG. 3 panel A top). In contrast, NKG2D ligand-negative monocytes from MNG patients induced only background degranulation and cytokine production when co-cultured with autologous IL-2-activated NK cells. Similar results were obtained when purified circulating monocytes from GBM and MNG patients were co-cultured with the NK cell line, NKL (FIG. 3 panel A, center). Antibody blocking of NKG2D significantly decreased cytokine production and degranulation of NKL cells in response to GBM patient-derived monocytes, although degranulation over background suggests additional activating receptor/ligand interactions (FIG. 3 panel A, bottom). TIMCs had a similar effect on NK cell functions (FIG. 3 panel B). In four GBM patients analyzed, 30.26% (SD+/−3.75%) of IL-2-activated peripheral blood NK cells degranulated in response to autologous TIMCs, as compared to 26.4% (SD+/−4.68%) that degranulated in response to U87 cells. The addition of both U87 cells and GBM TIMCs increased the percentage of degranulating NK cells (FIG. 3 panel C), indicating that NKG2D ligand-expressing TIMCs do not prevent NK cell activation. NK cells from healthy donors induced apoptosis of autologous monocytes only after exposure of the monocytes to U87 cell-free supernatant, and NK cell degranulation was significantly reduced by antibody blocking of NKG2D (FIG. 3 panel D). Increasing the ratio of NKG2D ligand-expressing monocytes to NK cells increased the percentage of degranulating NK cells (FIG. 3 panel E), and decreased NKG2D expression on NK cells (FIG. 3 panel F), suggesting that NKG2D ligand-expressing myeloid cells can be targets for NK cells in the tumor microenvironment. Addition of NKG2D ligand-expressing monocytes to NK and tumor cell co-cultures demonstrated that NKG2D ligand-bearing monocytes are recognized as well as U87 cells, significantly reducing the percentage of apoptotic U87 cells (FIG. 3 panel G).

Although these in vitro assays demonstrate that IL-2-activated NK cells can kill NKG2D-ligand bearing autologous monocytes, the existence of both NKG2D ligand-expressing myeloid cells and glioblastoma cells in patients indicates that the NK cells do not eliminate these potential target cells. Therefore, NK cells freshly isolated from patients were investigated. NK cells isolated from the GBM microenvironment lacked intracellular perforin and granzyme B as compared to NK cells isolated from circulation (FIG. 3 panel H). The amount of perforin mRNA increased in both circulating and tumor-associated NK cells in the presence of IL-2; however, tumor associated NK cells transcribed over 100-fold less perforin mRNA than untreated circulating NK cells from the same patients (FIG. 3 panel I). The amount of intracellular perforin increased in tumor-associated NK cells over the course of 72 hours of culture with IL-2, but never reached that expressed by untreated autologous circulating NK cells (FIG. 3 panel J). Culture of circulating and tumor-associated NK cells with NKG2D ligand-expressing tumor targets induced significant degranulation by both NK cell populations; however, tumor-associated NK cells lacked intracellular perforin as compared to circulating NK cells, so were less efficient killers (FIG. 3 panel K).

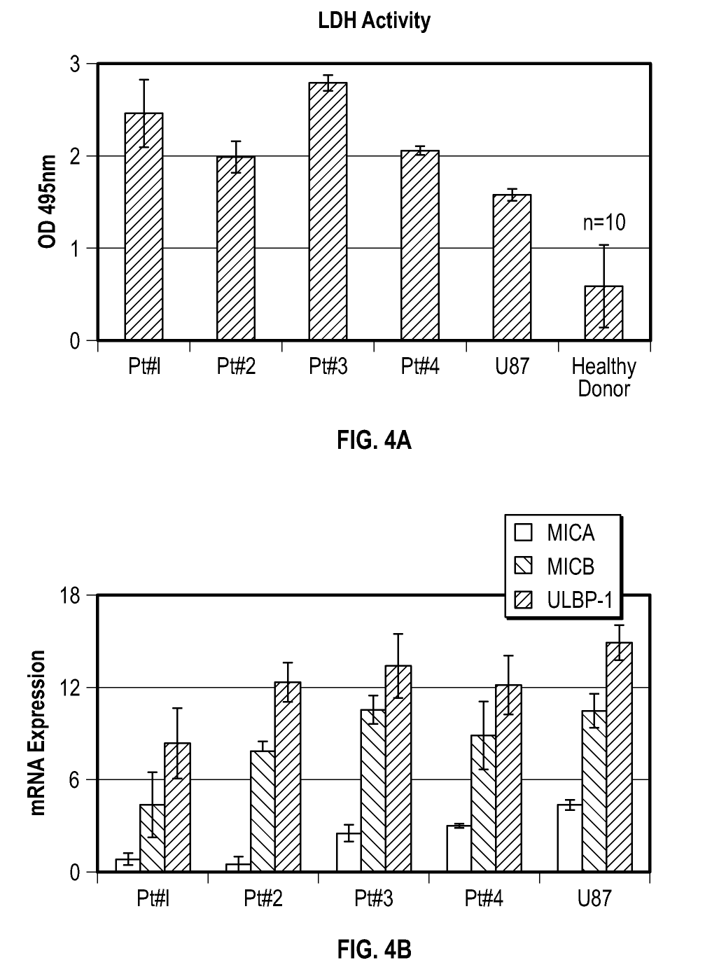

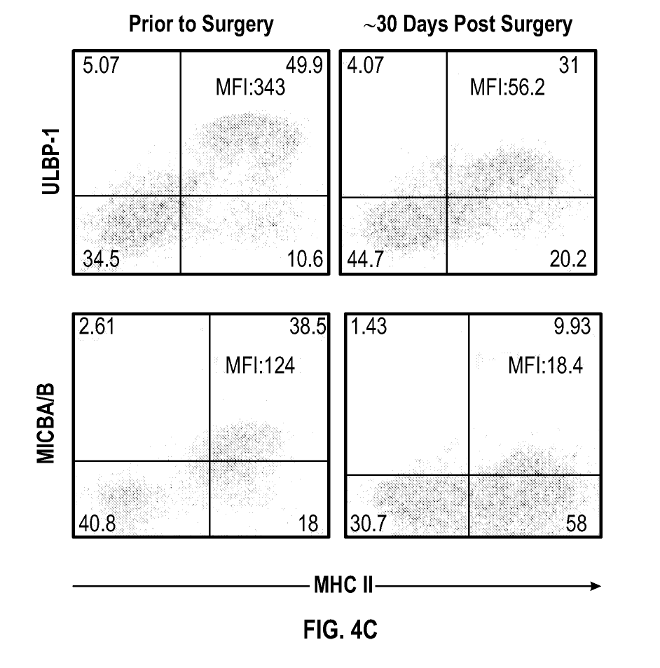

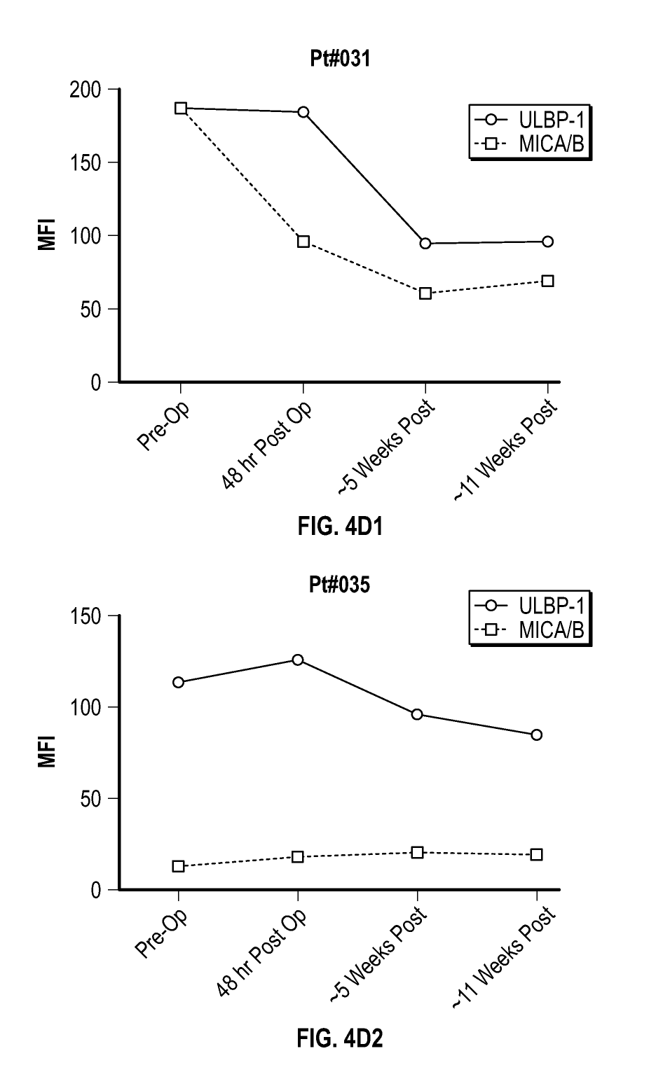

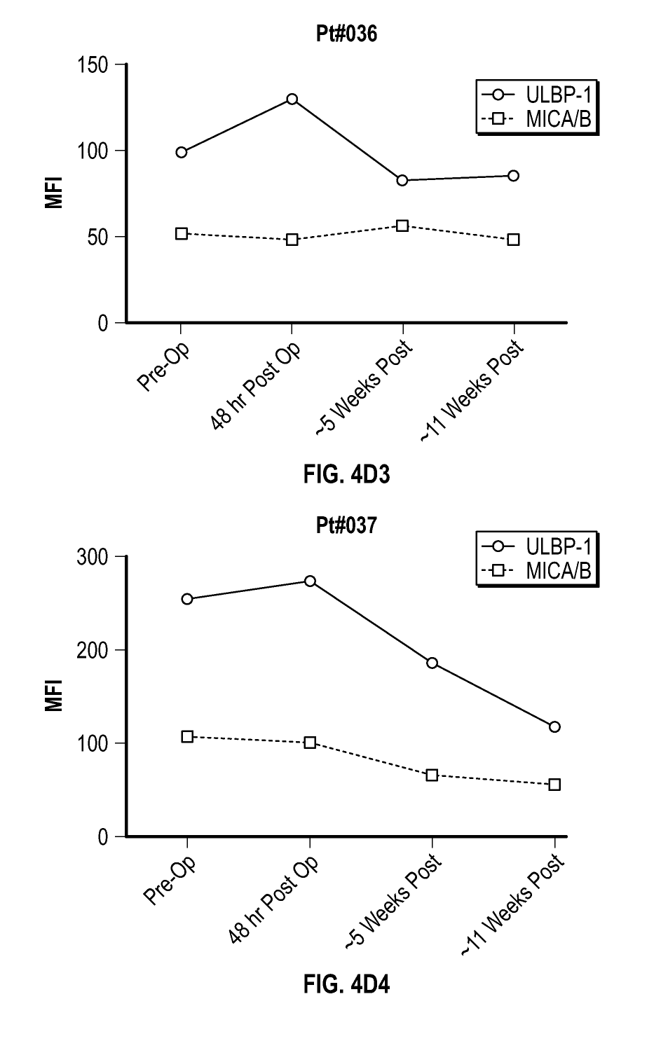

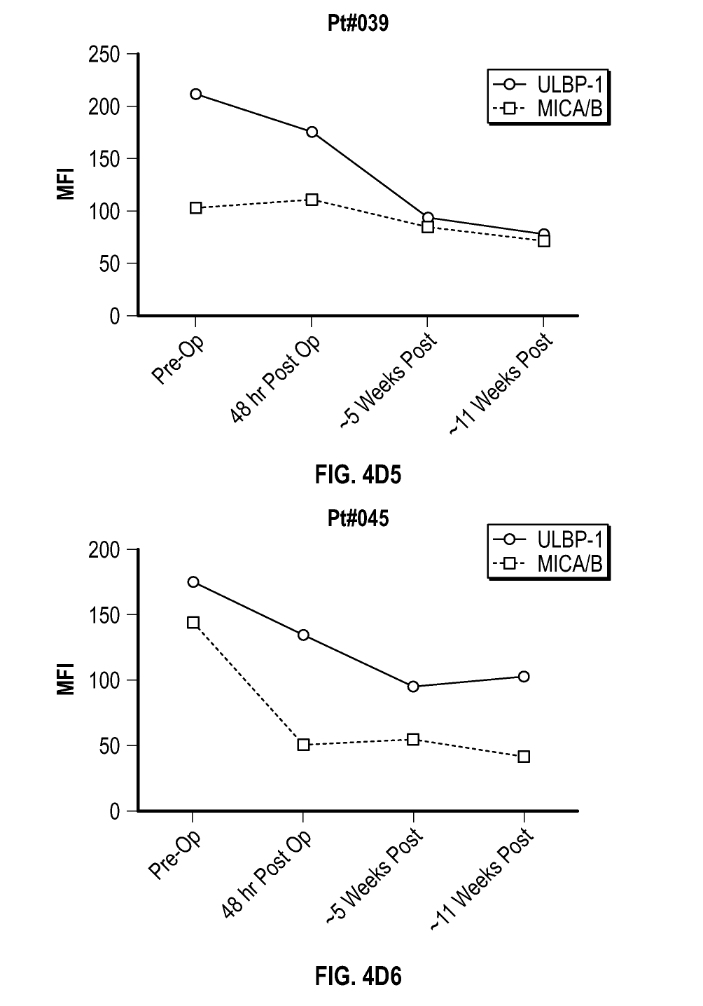

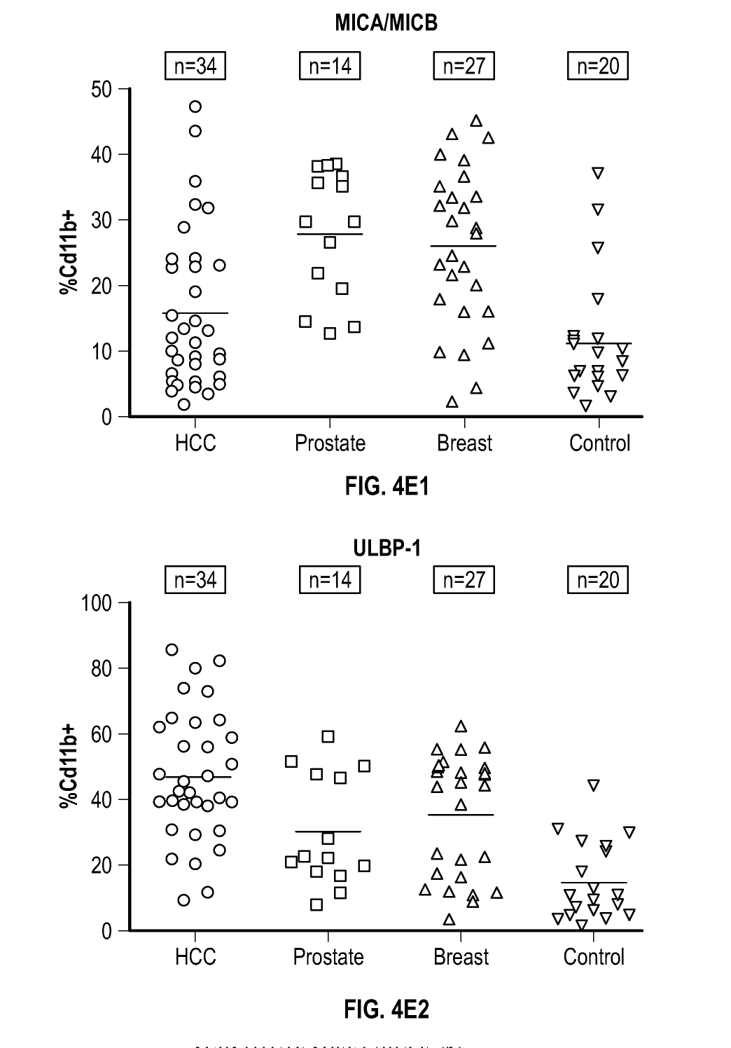

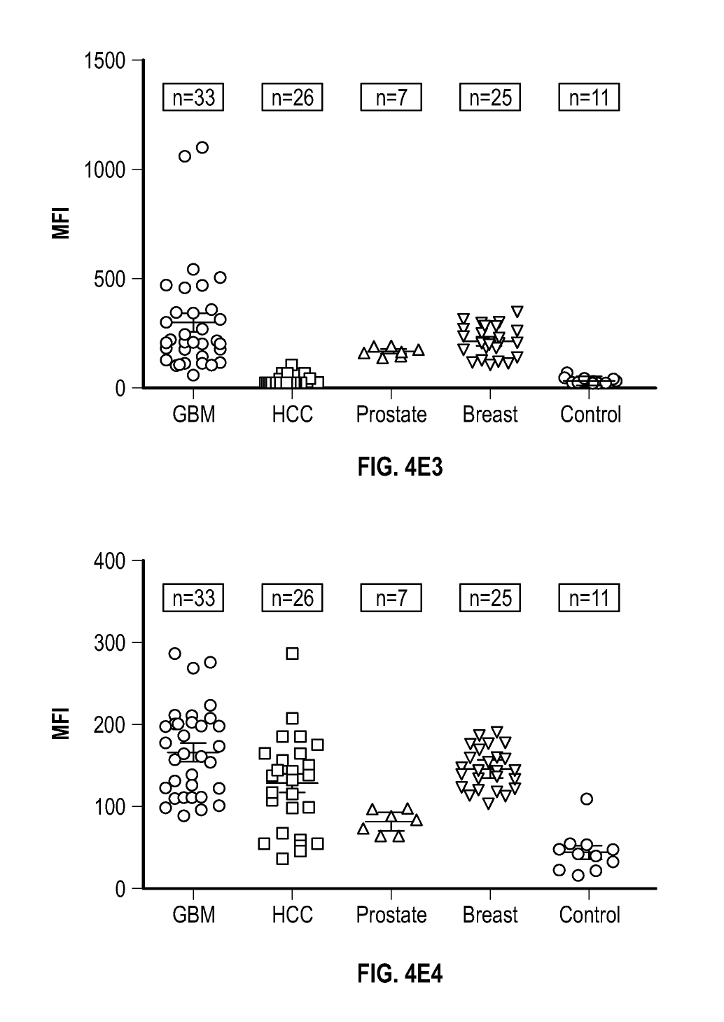

The LDH activity was measured in freshly isolated GBM patient sera due to activity loss after freezing (FIG. 12). The patient sera analyzed contained greater total LDH activity than U87 supernatant (FIG. 4 panel A). When cultured with MHD, GBM patient sera augmented transcription of MICB (6.37-14.54-fold over media alone) and ULBP-1 (10.34-22.32-fold over media alone) (FIG. 4 panel B), demonstrating that the LDH in patients' sera is sufficient to induce NKG2D ligand expression. GBM patients examined before and after tumor resection and at 34.8 days (+/−5.1 days) had significant reductions in the mean fluorescence intensity of NKG2D ligand staining on circulating monocytes (FIG. 4 panel C and 4 panel D), suggesting that ligand expression is dependent on tumor burden.

The presence of NKG2D ligand-bearing monocytes in the peripheral blood of GBM patients demonstrates that NKG2D ligands on myeloid cells are not restricted to the tumor site. Monocytes from patients with hepatocellular carcinoma (HCC, n=34), prostate cancer (n=14), and breast cancer (n=27) were then examined. Significant percentages of circulating monocytes in a subset of these patients expressed NKG2D ligands when compared to healthy controls (FIG. 4 panel E).

These findings revealed a novel mechanism of immunosuppression by tumors that can lead to immune escape. It was proposed that tumor-derived LDH-5 acts systemically by inducing expression of NKG2D ligands on the host's circulating monocytes, which in turn interacts with NKG2D receptors on NK cells, resulting in downregulation of the receptor (Crane, C. A, et al. TGF-beta downregulates the activating receptor NKG2D on NK cells and CD8+ T cells in glioma patients. Neuro-oncology 12, 7-13 (2010)) and inactivation of the NKG2D pathway in NK cells, a finding previously demonstrated in animal models with chronic exposure to NKG2D ligands (Oppenheim, D. E., et al. Sustained localized expression of ligand for the activating NKG2D receptor impairs natural cytotoxicity in vivo and reduces tumor immunosurveillance. Nat Immunol 6, 928937 (2005)). Although elevated serum LDH-5 correlates with poor prognoses in a variety of cancers, the mechanisms responsible for this are poorly understood (Kustner, W. & Weinreich, J. Comparative studies of lactate dehydrogenase isoenzyme patterns in the serums, tumors and metastases of tumor patients. Verhandlungen der Deutschen Gesellschaft fur Innere Medizin 75, 529-532 (1969)). In addition to mechanisms such as TGFβ and exome-associated soluble NKG2D ligand secretion, it was proposed that tumor-derived LDH-5 promotes immune suppression by inducing NKG2D ligands on myeloid cells that impair NKG2D-dependent NK cell immunity.

It was found that primary tumor cells in GBM patients express NKG2D ligands, even though the frequency of NK cells in the tumor is similar to that in peripheral blood (FIG. 5 Panel A). However, the level of NKG2D receptor on these NK cells was lower than observed in healthy control individuals (FIG. 5 Panel B). Myeloid cells were the most prevalent leukocytes infiltrating tumors in GBM patients (FIG. 5 panel C); 57.6% (n=18) of CD45⁺ cells among GBM tumor-infiltrating leukocytes were MHC class CD11b^{high} myeloid cells compared with 28.28% (n=13) in patients with benign intracranial meningioma (MNG) (FIG. 5 panel C). In GBM patients, tumor-infiltrating myeloid cells (CD11b^{high} macrophages and CD11b^{low} CNS-resident microglia) and circulating monocytes in the peripheral blood expressed at least two of the NKG2D ligands, MICB and ULBP-1 (n=33), which were not detected on myeloid cells in MNG patients (n=16) (FIG. 1 panels A and B). Patients with newly diagnosed GBM who were analyzed before receiving any therapy consistently expressed MICB and ULBP-1 on circulating monocytes, indicating that expression was independent of surgery, chemotherapy, radiation, or steroid treatment (FIG. 6, n=4). Transcriptional analysis revealed cell-intrinsic expression of MICB and ULBP-1 in GBM patients' circulating monocytes (n=19) (FIG. 5 panel C), excluding the possibility that soluble NKG2D ligands were passively acquired from the patients' sera. Of the eight NKG2D ligands evaluated by quantitative RT-PCR (qRT-PCR), only ULBP-1 and MICB were consistently expressed by monocytes and TIMCs in all GBM patients (FIG. 5 panel C). Acid washing of the patients' monocytes removed β2-microglobulin, a surface protein lacking a membrane anchor, but did not remove MICB or ULBP-1, confirming that these NKG2D ligands are expressed endogenously (FIG. 7).

Given their expression on circulating monocytes, it was hypothesized that the NKG2D ligands were induced by tumor-derived soluble factors that were acting systemically in the patients. By using cell-free supernatant or Transwell assays, it was determined that soluble products derived from the U87 glioma cell line induced expression of MICB and ULBP-1 on primary monocytes from healthy blood donors (FIG. 1 panel D). Heat denaturation (FIG. 1 panel E) and size-exclusion dialysis (FIG. 1 panel F) revealed that a heat labile factor of >10 kDa induced MICB and ULBP-1.

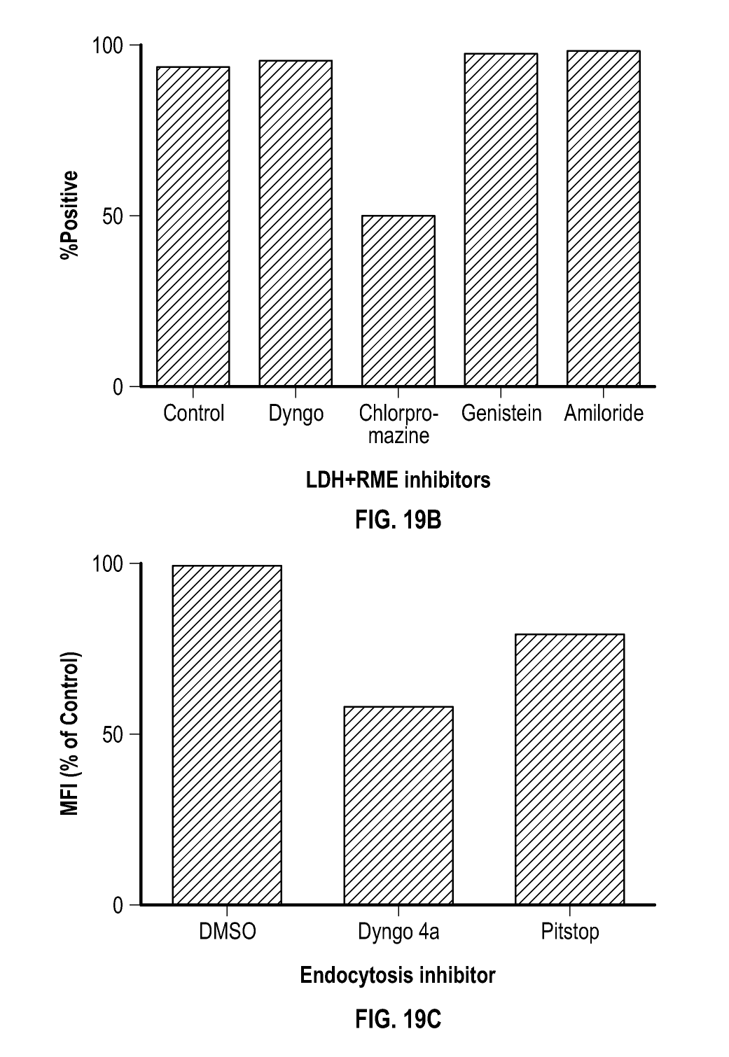

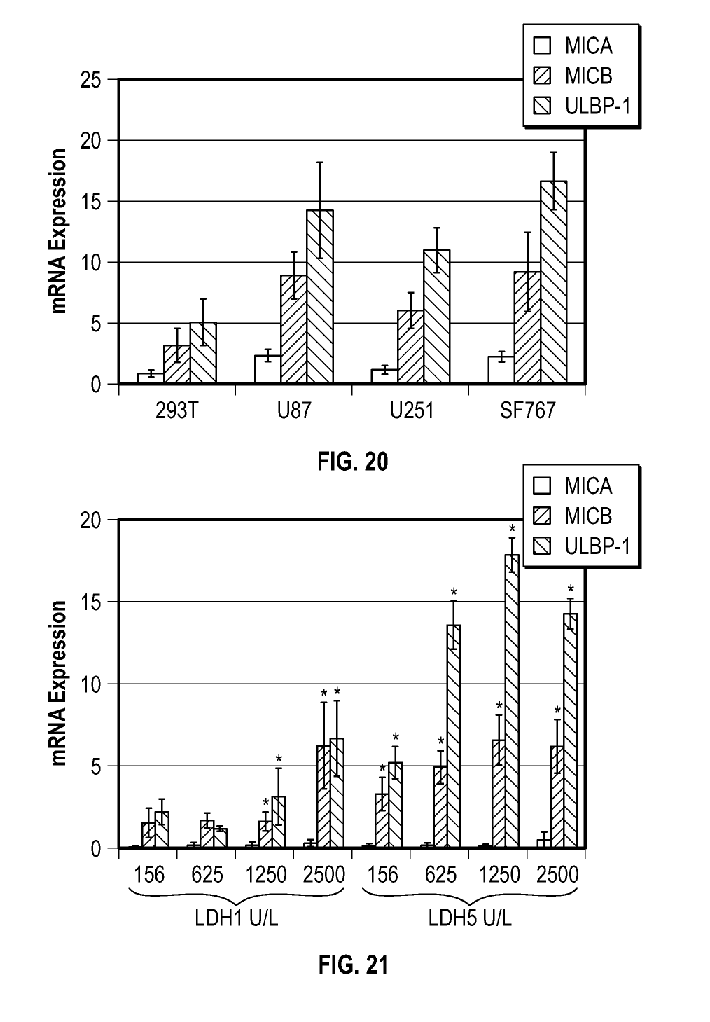

By using an unbiased protein purification screening strategy to determine the factor(s) responsible for in vitro induction of MICB and ULBP-1 transcription in healthy monocytes, LDH5 was identified as sufficient to induce NKG2D ligand expression in monocytes (FIG. 2 panels A and B). LDH is a tetrameric metabolic enzyme that binds pyruvate and promotes ATP production in resource-deprived environments. There are five isoforms of LDH, consisting of different ratios of α and β subunits, with varied tissue distribution. Consistent with prior reports of correlations between elevated LDH5 in sera of cancer patients and poor prognoses (13 [symbol] [symbol] -16), these findings indicated that LDH5 may promote immune escape of tumor cells by inducing NKG2D ligands on host myeloid cells, thereby subverting the antitumor activity. Glioma cell lines U87, U251, and SF767 transcribe LDH-A and LDH-B (FIG. 2 panel C), suggesting that they can make all five isoforms of LDH. These glioma cell lines secrete enzymatically active LDH into their supernatants (FIG. 2 panel D) that is sufficient to induce MICB and ULBP-1 mRNA in healthy monocytes (FIG. 20).

NKG2D ligand mRNA expression following treatment with purified, native LDH5 and U87 supernatant in the presence of sodium oxamate, a pyruvate analog that blocks LDH enzymatic activity was evaluated. Sodium oxamate significantly reduced the ability of U87 supernatant to induce NKG2D ligand mRNA expression in monocytes (FIG. 2 panel E). NKG2D ligand induction required LDH5 enzymatic activity and the presence of its substrate pyruvate (FIG. 2 panel F). Using purified, native LDH5 isolated from human liver, it was found that concentrations of enzymatically active LDH5 as low as 156 U/L significantly induced gene expression of MICB (7.85-fold induction) and ULBP-1 (13.26-fold induction) in monocytes compared with medium alone [healthy individuals have LDH5 serum concentrations of <150 U/mL (17)]. Higher concentrations of LDH5 (up to 625 U/L) induced a maximal increase in the amount of NKG2D ligand transcription. Interestingly, concentrations above 625 U/L induced less NKG2D ligand transcription, despite consistent cell viability, suggesting that high LDH5 concentrations may exhaust substrate or inhibit NKG2D ligand transcription (FIG. 2 panel G). LDH1, an isoform containing only β subunits, induced transcription of NKG2D ligands in monocytes, although greater concentrations of LHD1 were needed, and the amount of ligands expressed never reached that observed with LDH5 treatment (FIG. 21). Transfection of 293T cells with cDNA expression vectors encoding LDH-A, LDH-B, or both LDH-A and LDH-B induced the transcription of MICB and ULBP-1 in healthy monocytes, and NKG2D ligand transcription increased if both subunits were present, potentially producing all five isoforms of LDH (FIG. 2 panel J) and thus supporting redundant activity of one or more isoforms of LDH.

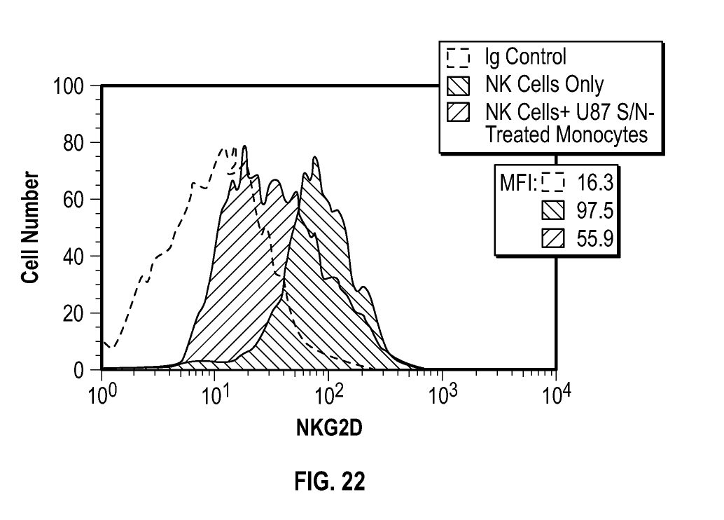

Having confirmed NKG2D ligand expression on the circulating monocytes in GBM patients and having identified a key tumor-derived enzyme responsible for transcriptional induction of NKG2D ligands on healthy monocytes, we considered the functional consequences of NKG2D ligand expression by monocytes. Because freshly isolated NK cells from the GBM patients demonstrate low expression of NKG2D and impaired NKG2D-dependent function (FIG. 22), the patients' NK cells were cultured overnight in IL-2 to restore NKG2D expression and function and then were co-cultured with autologous NKG2D ligand-bearing monocytes. As shown in FIG. 3 panel A, co-culture of the IL-2-activated NK cells with autologous NKG2D ligand-bearing monocytes triggered degranulation of 75.3% of NK cells and induced IFN-γ production by 20.2% of the degranulating NK cells. In contrast, NKG2D ligand-negative monocytes from MNG patients did not induce degranulation and cytokine production when co-cultured with autologous IL-2-activated NK cells. Similar results were obtained when purified circulating monocytes from GBM and MNG patients were co-cultured with the NK cell line, NKL (FIG. 3 panel A). Antibody blocking of NKG2D significantly decreased cytokine production and degranulation of NKL cells induced by NKG2D ligand-bearing monocytes derived from GBM patients, although degranulation over background in the NKG2D-blocked NK cells suggests the participation of additional activating receptor-ligand interactions (FIG. 3 panel A).

IL-2-activated NK cells from GBM patients also responded against autologous TIMCs. In four GBM patients analyzed, 30.26% (SD±3.75%) of IL-2-activated peripheral blood NK cells degranulated in response to autologous TIMCs, compared with 26.4% (SD±4.68%) that degranulated in response to U87 tumor cells used as a positive control (FIG. 3 panel B). The addition of both U87 cells and GBM TIMCs increased the percentage of degranulating NK cells (FIG. 3 panel C), indicating that TIMCs expressing NKG2D ligand do not prevent NK cell activation.

The ability of IL-2-activated NK cells from healthy blood donors to induce apoptosis of autologous monocytes before and after exposure to U87 glioblastoma cell supernatant to induce NKG2D ligands was tested. NK cells induced apoptosis of autologous monocytes only after exposure to U87 supernatant, which contained LDH. This induction was reduced significantly in the presence of a NKG2D-blocking antibody (FIG. 3 panel D). The addition of NKG2D ligand-expressing monocytes to co-cultures of NK and U87 tumor cells demonstrated that autologous NKG2D ligand-bearing monocytes were killed more readily than U87 tumor cells and significantly reduced the percentage of apoptotic U87 cells (FIG. 3 panel G).

Because LDH5 activity is lost after freezing, the LDH activity in sera that was freshly isolated from four GBM patients was measured. The amount of LDH measured in the patients' sera was greater than in supernatants harvested from the U87 tumor cell line used as control (FIG. 4 panel A). Importantly, when cultured with healthy monocytes, the GBM patient sera induced transcription of MICB (6.37- to 14.54-fold over medium alone) and ULBP-1 (10.34- to 22.32-fold over medium alone) (FIG. 4 panel B), suggesting that the LDH in the patients' sera is able to induce NKG2D ligand expression on healthy monocytes. In longitudinal studies, we observed that the mean fluorescent intensity of ULBP-1 staining of monocytes isolated from all GBM patients declined after tumor resection (FIG. 4 panels C and D); interestingly, however, in four of the six patients MICB, which was expressed in lower amounts, remained unchanged. In studies examining circulating monocytes in patients with other cancers, NKG2D ligands were detected on subsets of patients with hepatocellular carcinoma (n=34), prostate cancer (n=14), or breast cancer (n=27) (FIG. 4 panel E).

### LDHA Antibody Blocks LDH5 Uptake.

As shown in FIGS. 23-25, experiments were performed to show that an antibody specific for LDH5 can prevent LDH5 update in CD14+ cells. In an experiment utilizing the antibody with LDH5 in serum, the LDH5 complexed to the antibody was added to macrophages and to a sample of monocytes. As shown in FIG. 23, the mean fluorescence intensity was decreased in cells that were given the anti-LDH5 in combination with LDH5, indicating that the antibody blocked uptake when compared to the control cells, in which only the LDH5 was added to the cells. As shown in FIG. 24, the inhibitor chlorpromazine blocks LDH5 uptake. Monocyte derived macrophages were pre-incubated with 25 uM chlorpromazine for 30 min. Fluorescently labeled LDH5 was then added for an additional 30 min. Cells were then trypsinized, acid washed, fixed in 2% PFA. Fluorescence intensity was obtained by flow cytometry. As shown in FIG. 25, Malate Dehydrogenase (MDH) also blocks LDH5 uptake in CD14+ cells from healthy donors. Fluorescently labeled LDH5 was incubated with U87 in the presence or absence of equimolar MDH for 30 min. Cells were then trypsinized, acid washed, fixed in 2% PFA. Fluorescence intensity was obtained by flow cytometry. Cells exposed to LDH5-A488 were compared to cells without and amount of fluorescence was determined to represent amount of LDH5 inside of the cell.

### U87 Uptake.

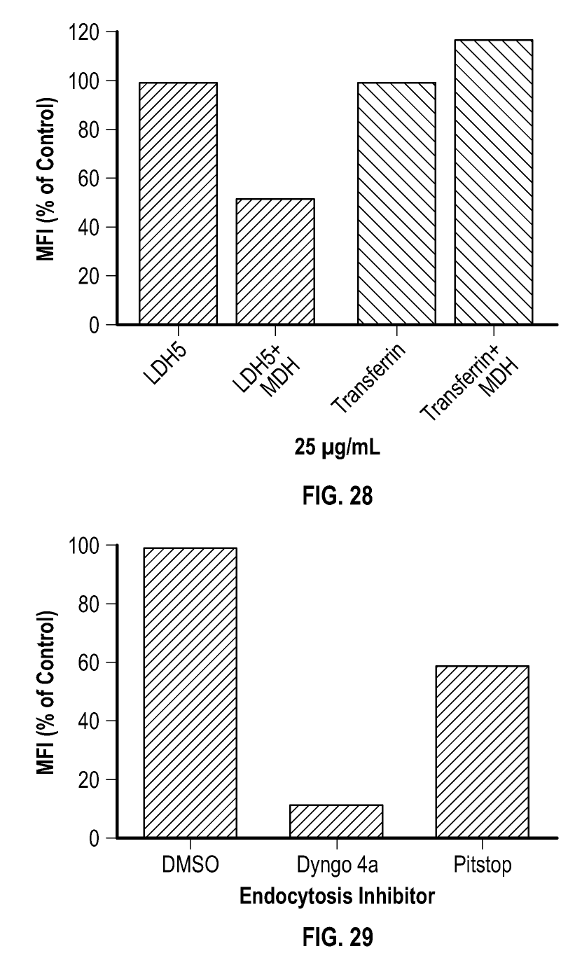

Experimental wells were pulsed with 10 ug/mL LDH-A488 for 30 min or 24 hours as indicated (control wells received no LDH) at 37° C. Cells were treated for 2 min with 0.5M acetic acid+150 mM NaCl (pH 2.5), followed by 4 subsequent washes in RPMI+10% FBS. It was demonstrated that internalization is mediated by a specific receptor that is reduced by blocking uptake using a monoclonal antibody to LDH5, and MDH, which has been demonstrated to compete with LDH5 for receptor binding. (See FIGS. 26-29).

### U87 and Mitochondrial Function.

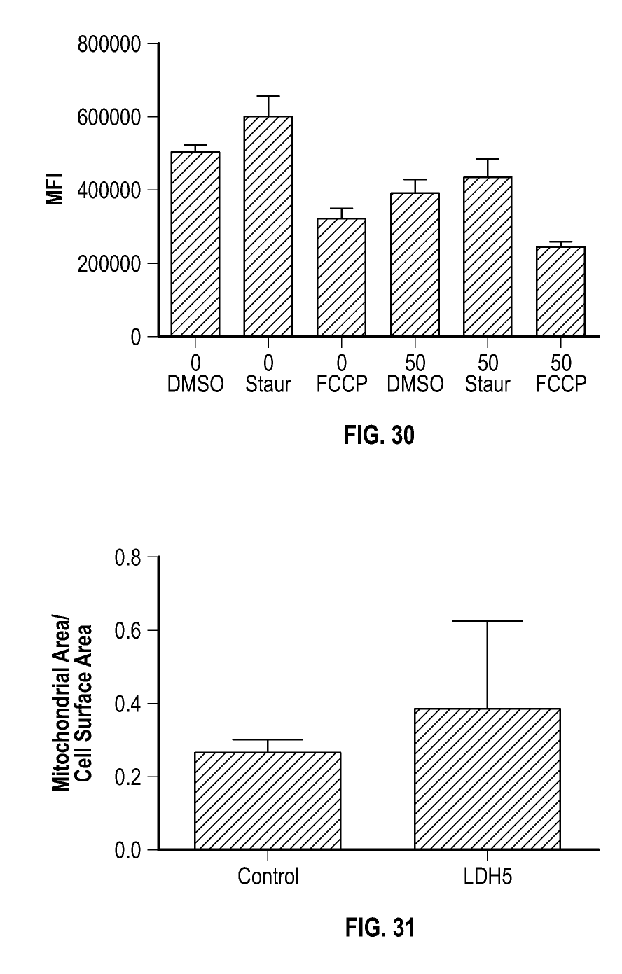

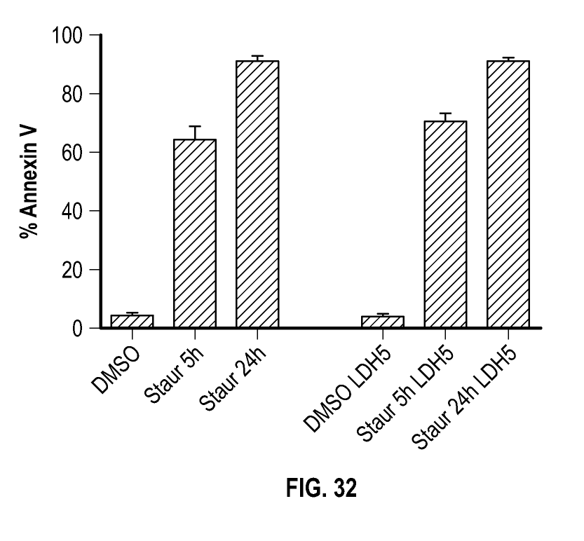

Experimental wells were pulsed with 50 ug/mL LDH-A488 for 24 hours as indicated (control wells received no LDH) at 37° C. Cells were treated for 2 min with 0.5M acetic acid+150 mM NaCl (pH 2.5), followed by 4 subsequent washes in RPMI+10% FBS. It was demonstrated that internalization results in mitochondrial membrane polarization (FIG. 30) without a significant reduction in mitochondrial number (FIG. 31) or cell death (FIG. 32). (See FIGS. 31-32).

### T Cells and NK Cells Internalize LDH5.

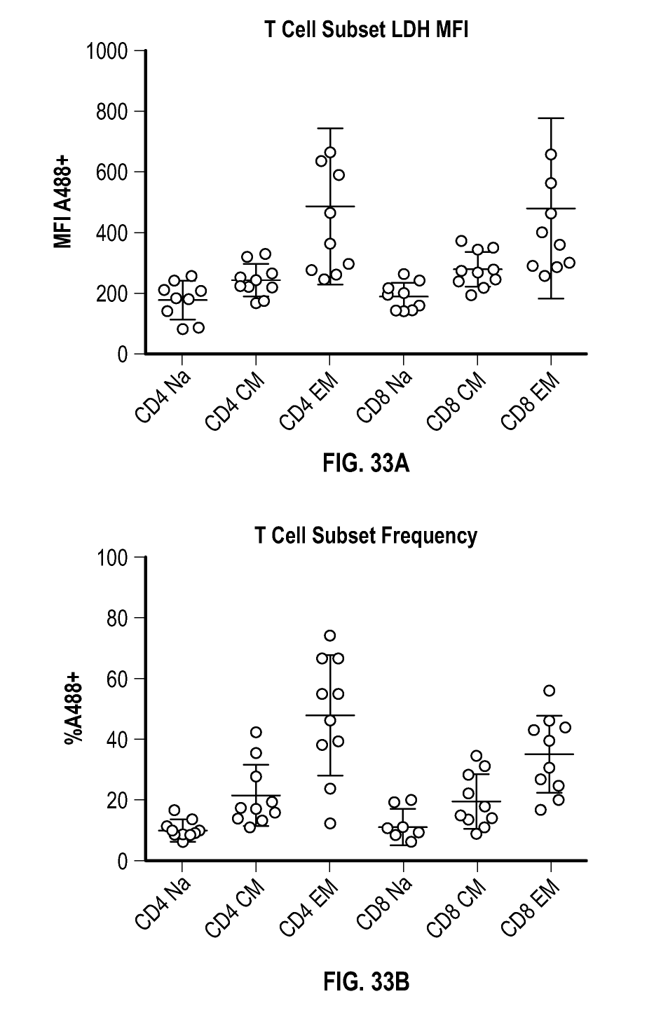

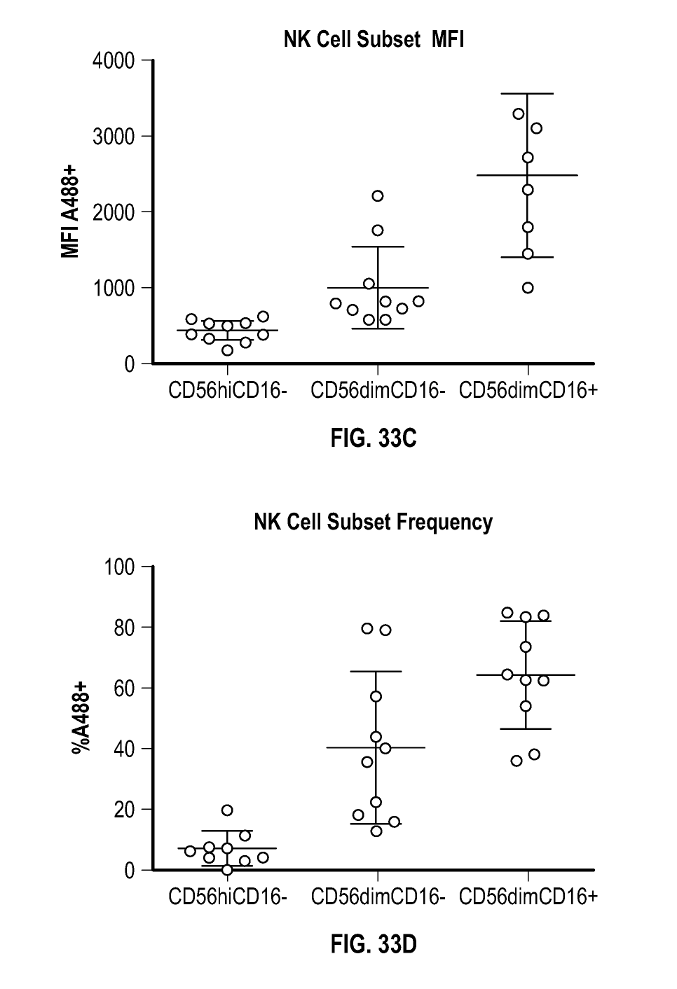

As shown in FIG. 33, T cells and NK cells internalize LDH5. Human PBMC were isolated from healthy human blood via Ficoll gradient. PBMC were cultured for 24 h in RPMI+10% FBS supplemented with 1000 U/mL IL-2. Experimental wells were pulsed with 10 ug/mL LDH-A488 for 30 min (control wells received no LDH) at 37° C. Cells were treated for 2 min with 0.5M acetic acid+150 mM NaCl (pH 2.5), followed by 4 subsequent washes in RPMI+10% FBS. Cells were stained with antibodies directed against CD4, CD8, CD28, and CD95 to delineate T cell subsets (CD28+CD95−, Naïve, Na; CD28+CD95+, central memory, CM; CD28−CD95+, effector memory, EM) or against CD3, CD56, and CD16 to delineate NK cell subsets (CD3−CD56hiCD16−; CD3−CD56dimCD16−; CD3−CD56dimCD16+). Frequency and MFI of A488 was corrected against control (no LDH pulse). Shown are 10 data points: 2 patients on 2 different days (in duplicate), and 1 patient on one day in duplicate.

Time can have an Impact on LDH Uptake.

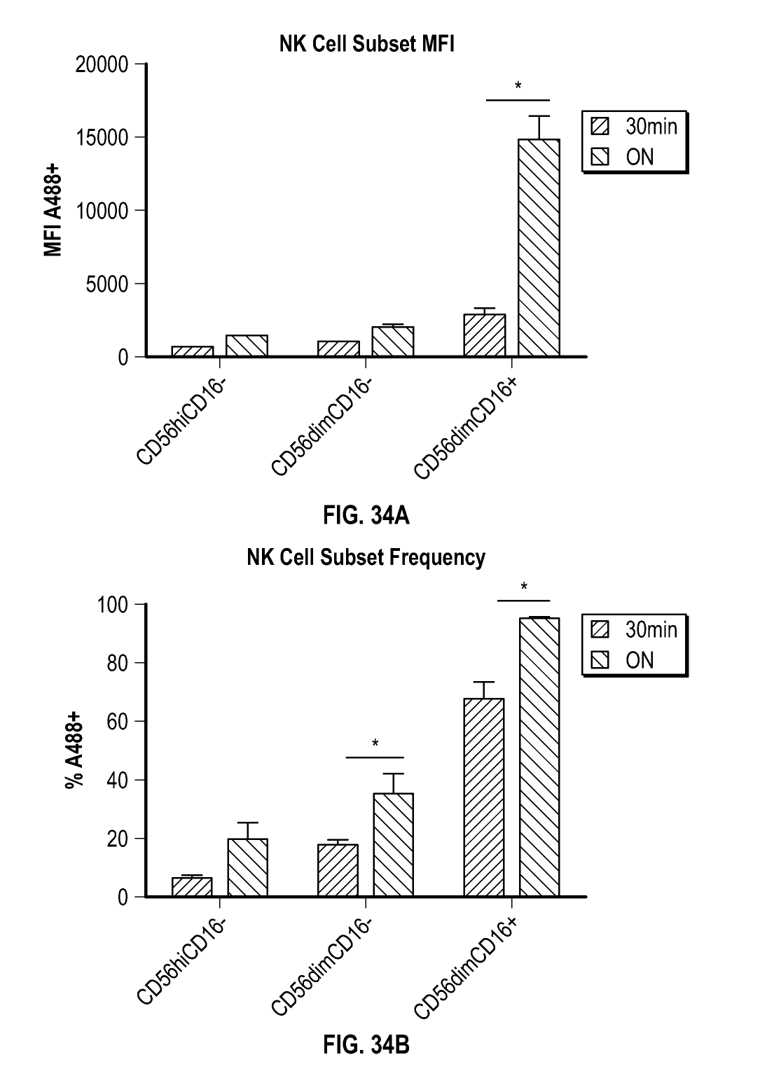

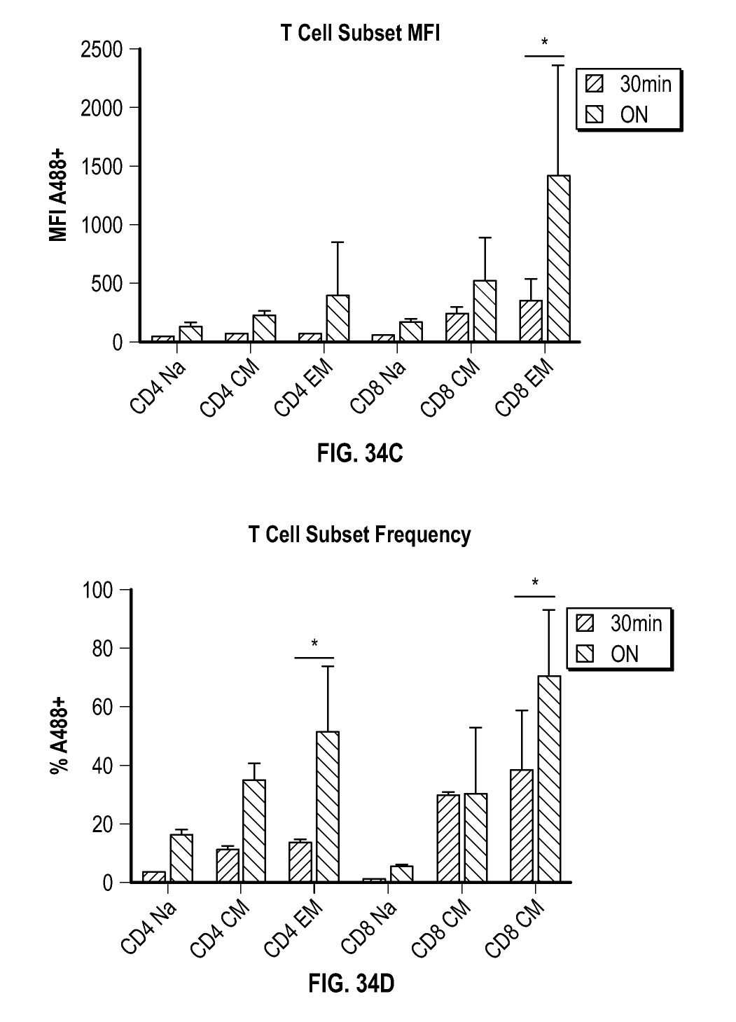

Human PBMC were isolated from healthy human blood via Ficoll gradient. PBMC were cultured for 24 h in RPMI+10% FBS supplemented with 1000 U/mL IL-2. Experimental wells were pulsed with 10 ug/mL LDH-A488 for either 30 min or 24 h at 37° C. Cells were treated for 2 min with 0.5M acetic acid+150 mM NaCl (pH 2.5), followed by 4 subsequent washes in RPMI+10% FBS. Cells were stained with antibodies directed against CD4, CD8, CD28, and CD95 to delineate T cell subsets (CD28+CD95−, Naïve, Na; CD28+CD95+, central memory, CM; CD28−CD95+, effector memory, EM) or against CD3, CD56, and CD16 to delineate NK cell subsets (CD3−CD56hiCD16−; CD3−CD56dimCD16−; CD3−CD56dimCD16+). Frequency and MFI of A488 was corrected against control (no LDH pulse). Shown are data from 2 patients in duplicate, each ran on a different day. (See FIG. 34). From these experiments, it is predicted that chronic exposure to LDH5 will have a significant impact on NK cell and T cell function, particularly if cytotoxic immune cells are activated, which could have serious negative consequences on treatments predicated on the activation of immune cells, such as checkpoint blockades, small molecule inhibitors, and adoptive cellular therapies.

### Activation of T Cells Impact LDH Endocytosis.

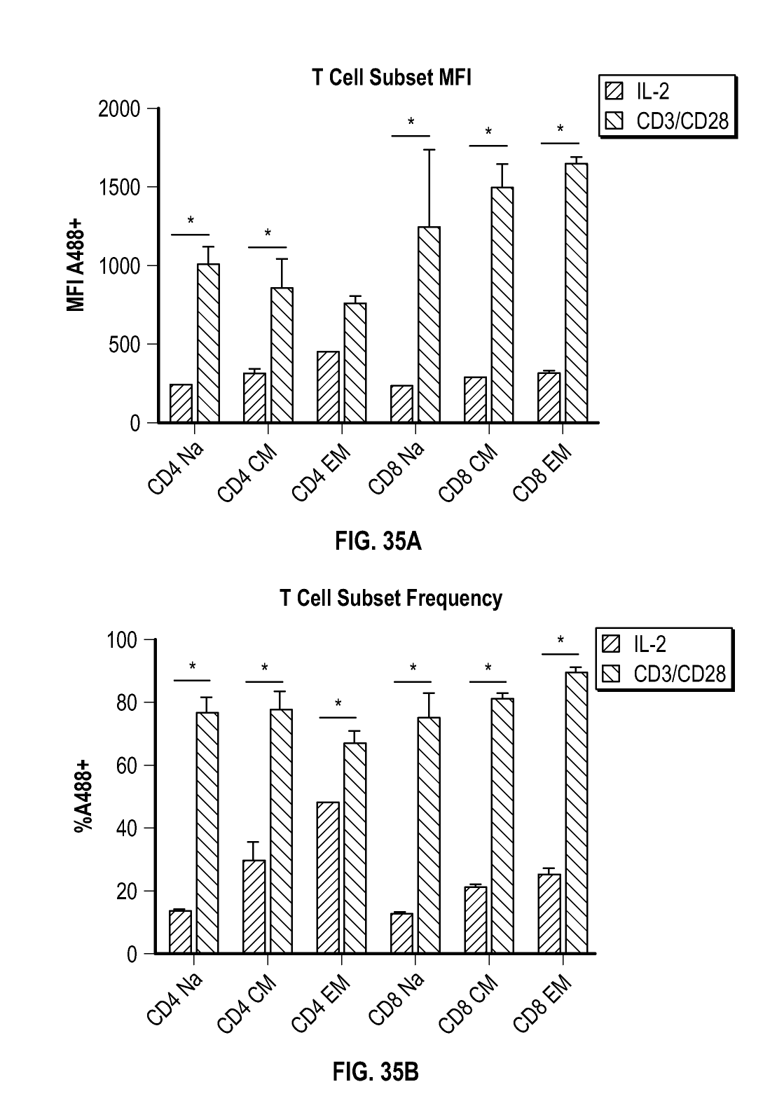

Human PBMC were isolated from healthy human blood via Ficoll gradient. T cells were separated from bulk PBMC via flow-through from a CD14+ selection kit (EasySep), followed by a Pan T cell Negative selection kit (Miltenyi). T cells were cultured for 24 h in RPMI+10% FBS and stimulated with either 1000 U/mL IL-2 or Human CD3/CD28 Dynabeads (Life Technologies). Activated cells were then pulsed with or without 10 ug/mL LDH-A488 for 30 min. Cells were treated for 2 min with 0.5M acetic acid+150 mM NaCl (pH 2.5), followed by 4 subsequent washes in RPMI+10% FBS. Cells were stained with antibodies directed against CD4, CD8, CD28, and CD95 to delineate T cell subsets (CD28+CD95−, Naïve, Na; CD28+CD95+, central memory, CM; CD28−CD95+, effector memory, EM) or against CD3, CD56, and CD16 to delineate NK cell subsets (CD3-CD56hiCD16−; CD3−CD56dimCD16−; CD3−CD56dimCD16+). Frequency and MFI of A488 was corrected against control (no LDH pulse). Shown is data from 2 patients in duplicate on same day. (See FIG. 35). From these experiments, there was internalization by T cells, and it is therefore predicted that the functional consequences will impact T cell responses to tumors. It is also predicted that chronic exposure to LDH5 will have a significant impact on NK cell and T cell function, particularly if cytotoxic immune cells are activated, which could have serious negative consequences on treatments predicated on the activation of immune cells, such as checkpoint blockades, small molecule inhibitors, and adoptive cellular therapies.

Understanding the tumor microenvironment in patients has the potential to improve experimental therapy design. In patients, the impact of the tumor microenvironment on the immune system is highly complex, and ex vivo observations are the result of the collective influence of many cell types and the proteins they produce. The in vitro study described herein, identifies a previously unidentified mechanism that may contribute to tumor immune escape in patients with GBM based on the observation that circulating monocytes express the activating ligands for the NK cell receptor, NKG2D. Although the relationship between tumor-derived LDH5 and NKG2D ligand expression on circulating monocytes in vivo will be only one of many consequences of the tumor on immune responses, several experiments described herein were performed and present several previously unreported findings that may provide insight into tumor immune escape in patients. Specifically, the data demonstrate: (i) expression of two ligands for NKG2D, MICB and ULBP-1, on circulating monocytes, a finding that extends to subsets of patients with breast, prostate, and HCV-induced hepatocellular carcinoma; (ii) NK cell degranulation in response to autologous NKG2D ligand-bearing monocytes and tumor-infiltrating myeloid cells; (iii) identification of an extracellular, tumor-derived, metabolic enzyme that is sufficient to induce transcription of MICB and ULBP-1; and (iv) in a small cohort of patients with recurrent GBM, a decrease in the amount of NKG2D ligand expression on circulating monocytes within 5 weeks of surgical reduction of the tumor, suggesting that NKG2D ligand expression is dependent on the presence of a tumor mass.

These findings that LDH induces NKG2D ligands on myeloid cells represents one of many mechanisms that tumors may use to disrupt immune surveillance dependent on the NKG2D pathway. For example, previous studies have demonstrated that TGF-β can decrease NKG2D expression on NK cells in vitro and therefore may contribute to decreased NKG2D expression on circulating NK cells in patients. Additional soluble tumor-derived proteins in addition to LDH may induce NKG2D ligand expression on myeloid cells. As such the biochemical purification strategy suggests that other, as yet unidentified, factors can induce NKG2D ligand expression on monocytes from healthy donors, and previous studies have described NKG2D ligand expression following DNA damage, viral infection, and heat shock.

The NKG2D pathway serves an important role in host defense against viral pathogens and cancer. Viruses have evolved specific mechanisms to evade recognition by NKG2D-bearing NK cells and T cells. For example, mouse and human cytomegalovirus possess several genes encoding viral proteins that target and degrade NKG2D ligands before they are displayed on the surface of infected cells. Similarly, the E3/19K protein encoded by adenovirus retains MICA and MICB within the cytoplasm of infected cells, leading to their degradation, and cowpox and monkeypox viruses produce a soluble antagonist of NKG2D. Prior studies have reported that tumors also may evade detection by NKG2D by releasing NKG2D ligand-containing exosomes or by secreting high amounts of TGF-β, which can block transcription of NKG2D receptor in T cells and NK cells. Here, it is shown that another mechanism whereby tumors can evade NKG2D-dependent immunity, which works systemically. In prior studies in which soluble NKG2D ligands have been detected in the sera of cancer patients, it has been assumed that the source of these ligands is the shedding of the proteins or exosomes by the tumor cells themselves. The unexpected findings, as described herein, include that these NKG2D ligands can be derived from host immune cells rather than the tumors. Prior studies have reported that cancer patients with elevated amounts of LDH in sera have a poor prognosis. Although this poor prognosis might simply reflect larger tumor burdens, the secretion of LDH also can contribute to the immune evasion of these cancers by its induction of NKG2D ligands on host myeloid cells. In a recent study, Husain et al. demonstrated that transfection of mouse tumor cells to overexpress LDH resulted in more aggressive tumor growth; however, in this study there was no evidence for induction of NKG2D ligands by host cells or for any modulation of the NKG2D receptor on the host's NK cells, implying another mode of action. Indeed, as shown in the experiments herein, there are no direct effects of LDH or its product lactate on NKG2D expression or function using human NK cell effectors.

It is hypothesized that the induction of MICB and ULBP-1 on host myeloid cells, both locally and systemically, by tumor-derived LDH provides a mechanism to subvert NK cell and CD8⁺ T-cell responses against the tumor. It has been demonstrated that chronic exposure of NK cells to NKG2D ligand-bearing cells in vivo and in vitro results in down-modulation of the NKG2D receptor on NK cells and inactivation of the NKG2D pathway (7, 26 [symbol] [symbol] [symbol] -30). Down-modulation of NKG2D on NK cells is mediated much more efficiently by cell surface-expressed NKG2D ligands than by soluble NKG2D ligands, likely because of the clustering and cross-linking of the NKG2D receptor by the cell membrane-associated NKG2D ligands. The ability of tumor-derived LDH to induce MICB and ULBP-1 on host myeloid cells systemically, as evidenced by detection of NKG2D ligand-bearing monocytes in the circulation of GBM patients in whom the tumor remains localized in the CNS, might down modulate NKG2D on NK cells even before their migration into the tumor site.

Tumor Cells Express LDH5 mRNA and Protein.

mRNA expression was examined in Glioblastoma cell lines. As shown, Glioblastoma cell lines produce LDH-5 subunit mRNA (FIG. 11, Panel A) and active LDH enzyme as seen by the absorbance at 495 nm (FIG. 11, Panel B). As shown, tumor cells express LDH5 mRNA and protein which supports that the LDH5 found in sera and patient samples is tumor derived.

LDH5 is Labile, Making it Important to Assay Monomers and not Just Activity in Patient Biological Fluids, or to Analyze Immediately, as Activity is Lost after Freezing Patient Sera.

As shown in FIG. 12, LDH-5 activity is significantly reduced following freeze/thaw. This indicates that LDH5 is labile, making it important to assay monomers and not just activity in patient biological fluids, or to analyze immediately, as activity is lost after freezing patient sera.

The Impact that Seen on Tumor Cells and Immune Cells is not the Result of Elevated Lactate.

As shown in FIG. 13, LDH-5 nor soluble factors produced by the glioblastoma cell line U87 is sufficient to elevate extracellular lactate. As shown, the impact that is seen on tumor cells and immune cells is not the result of elevated lactate, which comports with earlier discussion (Hussain et al, Journal of immunology, 2013).

Hypoxia Elevates LDH5 Expression, Suggesting that Autocrine Functions of LDH5 May be Important for Survival of Tumor Cells in Oxygen Deprived Environments.

As shown in FIG. 14, LDH-5 expression is up-regulated during hypoxia. As hypoxia elevates LDH5 expression, this suggests that autocrine functions of LDH5 can be important for survival of tumor cells in oxygen deprived environments.

Freshly Isolated Patient Sera LED to Elevated LDH5 in Circulation and was Correlated with an Impact on Monocytes.

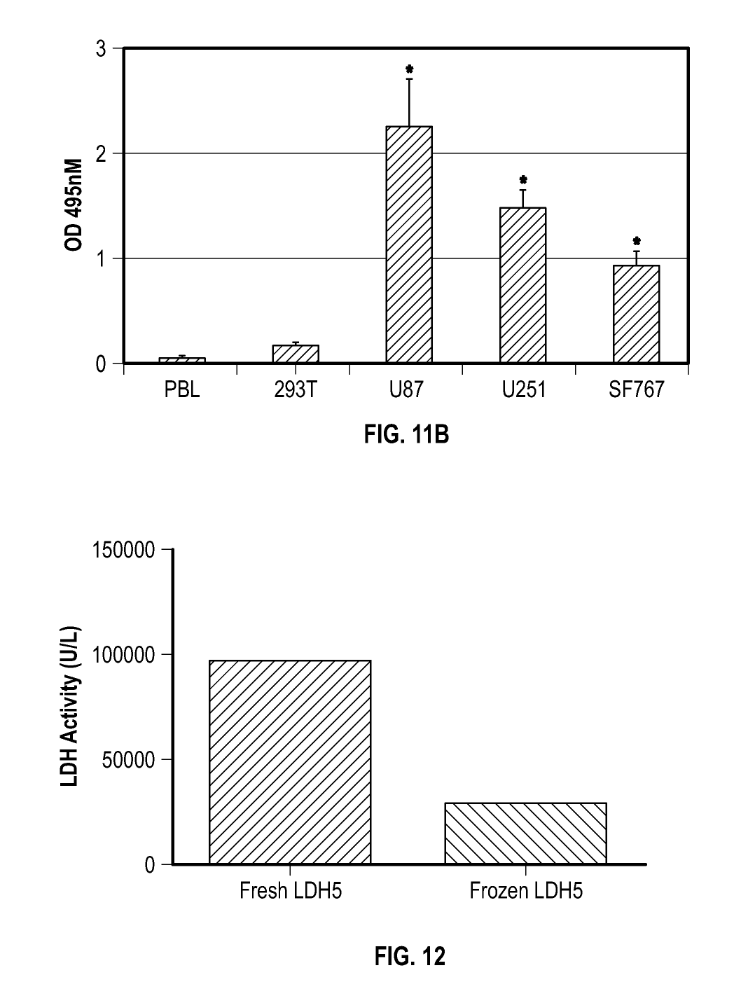

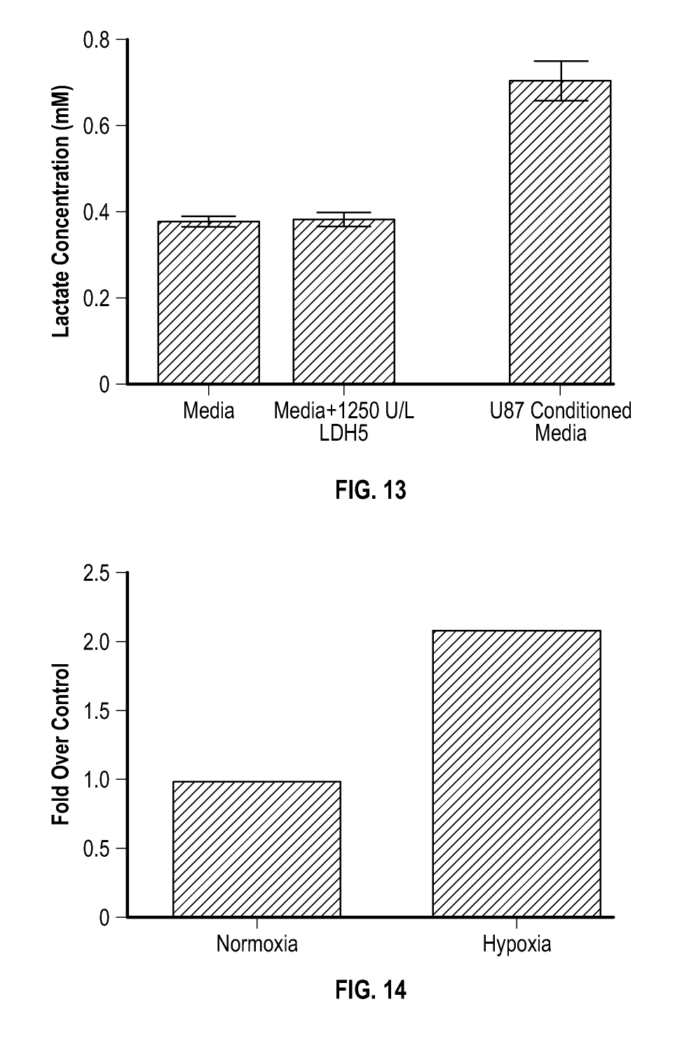

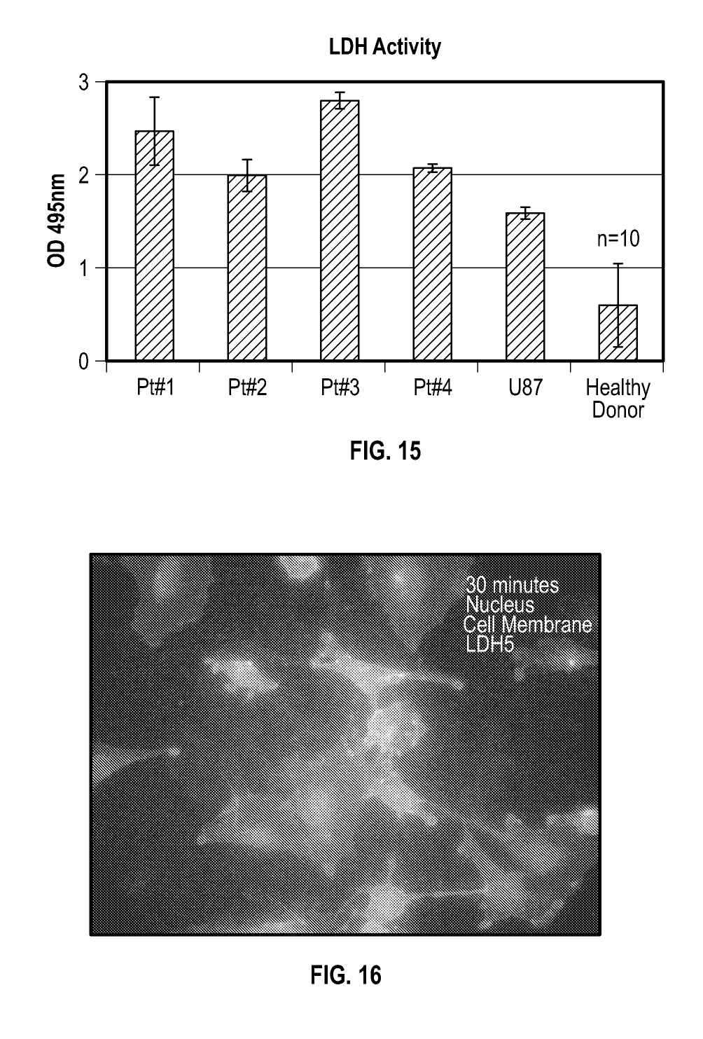

As shown in FIG. 15, patient sera was analyzed immediately after tumor resection (never frozen) and found to contain active LDH as compared to healthy donors. When freshly isolated (never frozen) patient sera were analyzed, elevated LDH5 was found in circulation, and this was correlated with the impact on monocytes.

LDH5 Internalization is Reduced with Receptor Mediated Endocytosis Inhibitors.

As shown in FIG. 19, LDH-5 internalization is receptor mediated and clathrin dependent. LDH5 internalization is reduced with receptor mediated endocytosis inhibitors. This supports that internalization is mediated by a specific receptor, as opposite to bulk phagocytosis.

It is understood that the examples and alternatives described herein are for illustrative purposes only and that various modifications or changes in light thereof will be suggested to persons skilled in the art and are to be included within the spirit and purview of this application and scope of any appended claims. All figures, tables, and appendices, as well as publications, patents, and patent applications, cited herein are hereby incorporated by reference in their entirety for all purposes.

1. A method of inhibiting, ameliorating, or treating cancer in a subject comprising:
selecting a subject to receive a binding agent specific for Lactate dehydrogenase 5 (LDH-5), or selecting a subject to receive an anti-cancer therapy;
providing to said subject an amount of a binding agent, which may be humanized, such as a fragment comprising a CDR domain or a Fab fragment, that is specific for LDH-5, preferably a binding agent that is specific for amino acid residues 1-241, 1-200, 1-175, 1-150, 1-125, 1-100, 1-75, 1-50, 1-25, 1-15, 1-10, or 1-5 or a range defined by any two numbers in between 1-241, including 1 and 241 of SEQ ID NOs 1, 2, 3 or 4, and
optionally, observing, analyzing, monitoring, or measuring an inhibition, amelioration, or treatment of cancer in said subject.

2.-51. (canceled)


SEQUENCE LISTING (24 sequences)

SEQ ID NO: 1 - protein, 50 aa, Artificial Sequence
  Fragment comprising amino acids 2-51 of human LDH-5
ATLKDQLIYNLLKEEQTPQNKITVVGVGAVGMACAISILMKDLADELALV

SEQ ID NO: 2 - protein, 241 aa, Artificial Sequence
  LDH-5
MATLKDQLIYNLLKEEQTPQNKITVVGVGAVGMACAISILMKDLADELALVDVIEDKLKGEMMDLQHGSLFLRTPKIVSGKDYNVTANSKLVIITAGARQQEGESRLNLVQRNVNIFKFIIPNVVKYSPNCKLLIVSNPVDILTYVAWKISGFPKNRVIGSGCNLDSARFRYLMGERLGVHPLSCHGWVLGEHGDSSVPVWSGMNVAGVSLKTLHPDLGTDKDKEQWKEVHKQVVERVFTE

SEQ ID NO: 3 - protein, 20 aa, Artificial Sequence
  Fragment comprising amino acids 2-21 of human LDH-5
ATLKDQLIYNLLKEEQTPQN

SEQ ID NO: 4 - protein, 30 aa, Artificial Sequence
  Fragment comprising amino acids 22-51 of human LDH-5
KITVVGVGAVGMACAISILMKDLADELALV

SEQ ID NO: 5 - DNA, 20 bp, Artificial Sequence
  Forward primer for ULBP-1
tgcaggccaggatgtcttgt

SEQ ID NO: 6 - DNA, 23 bp, Artificial Sequence
  Reverse primer for ULBP-1
catccctgttcttctcccacttc

SEQ ID NO: 7 - DNA, 20 bp, Artificial Sequence
  Forward primer for ULBP-2
cagagcaactgcgtgacatt

SEQ ID NO: 8 - DNA, 20 bp, Artificial Sequence
  Reverse primer for ULBP-2
ggccacaaccttgtcattct

SEQ ID NO: 9 - DNA, 20 bp, Artificial Sequence
  Forward primer for ULBP-3
ggatttcacacccagtggac

SEQ ID NO: 10 - DNA, 20 bp, Artificial Sequence
  Reverse primer for ULBP-3
gcctcttcttcctgtgcatc

SEQ ID NO: 11 - DNA, 20 bp, Artificial Sequence
  Forward primer for ULBP-4
ggctcagggaattcttaggg

SEQ ID NO: 12 - DNA, 20 bp, Artificial Sequence
  Reverse primer for ULBP-4
cattttgccaccagacacag

SEQ ID NO: 13 - DNA, 20 bp, Artificial Sequence
  Forward primer for ULBP-5
caagacagtcacacccgtca

SEQ ID NO: 14 - DNA, 20 bp, Artificial Sequence
  Reverse primer for ULBP-5
aagccatcctgtgcagtctc

SEQ ID NO: 15 - DNA, 20 bp, Artificial Sequence
  Forward primer for ULBP-6
tgacatcaccgtcatcccta

SEQ ID NO: 16 - DNA, 20 bp, Artificial Sequence
  Reverse primer for ULBP-6
tgctcacaagacatccttgc

SEQ ID NO: 17 - DNA, 21 bp, Artificial Sequence
  Forward primer for MICA
acaatgccccagtcctccaga

SEQ ID NO: 18 - DNA, 23 bp, Artificial Sequence
  Reverse primer for MICA
attttagatatcgccgtagttct

SEQ ID NO: 19 - DNA, 21 bp, Artificial Sequence
  Forward primer for MICB
tgagccccacagtcttcgtta

SEQ ID NO: 20 - DNA, 21 bp, Artificial Sequence
  Reverse primer for MICB
cctgcgtttctgcctgtcata

SEQ ID NO: 21 - DNA, 44 bp, Artificial Sequence
  LDH-A 5′ primer
cggccagaattccgccaccatggcaactctaaaggatcagctga

SEQ ID NO: 22 - DNA, 39 bp, Artificial Sequence
  LDH-A 3′ primer
cgccgactcgagtaataaaattgcagctccttttggatc

SEQ ID NO: 23 - DNA, 43 bp, Artificial Sequence
  LDH-B 5′ primer
cgccgaggtacccgccaccatggcaactcttaaggaaaaactc

SEQ ID NO: 24 - DNA, 39 bp, Artificial Sequence
  LDHB 3′ primer
cgccgagaattctaatcacaggtcttttaggtccttctg